SEQUENCE LISTING (288 sequences)

SEQ ID NO: 1 - protein, 15 aa, Apis mellifera
NKIDDCNYAIKRIAL

SEQ ID NO: 2 - protein, 14 aa, Apis mellifera
LPSFWIPSKTPEAK

SEQ ID NO: 3 - protein, 13 aa, Apis mellifera
KSLVGTPYWMSPE

SEQ ID NO: 4 - protein, 14 aa, Apis mellifera
QHAQKETEFLRLKR

SEQ ID NO: 5 - protein, 15 aa, Apis mellifera
TYGRTTKTFCGTPEY

SEQ ID NO: 6 - protein, 15 aa, Apis mellifera
PFQGDNIYKLYENIG

SEQ ID NO: 7 - protein, 15 aa, Apis mellifera
RRKSLVGTPYWMSPE

SEQ ID NO: 8 - protein, 15 aa, Apis mellifera
QELPRRKSLVGTPYW

SEQ ID NO: 9 - protein, 15 aa, Apis mellifera
LKGAILTTMLATRNF

SEQ ID NO: 10 - protein, 15 aa, Apis mellifera
IRSRPPYTDYVSTRW

SEQ ID NO: 11 - protein, 15 aa, Apis mellifera
QEDQQFVSLYGTEEY

SEQ ID NO: 12 - protein, 14 aa, Apis mellifera
PGERLSTWCGSPPY

SEQ ID NO: 13 - protein, 14 aa, Apis mellifera
LSTWCGSPPYAAPE

SEQ ID NO: 14 - protein, 15 aa, Apis mellifera
HRDIKSDSILLTADG

SEQ ID NO: 15 - protein, 15 aa, Apis mellifera
TPGNKLDTFCGSPPY

SEQ ID NO: 16 - protein, 12 aa, Apis mellifera
LYDAFISYSHKD

SEQ ID NO: 17 - protein, 15 aa, Apis mellifera
KRKSFIGTPYWMAPE

SEQ ID NO: 18 - protein, 15 aa, Apis mellifera
KNRSFAKSVIGTPEF

SEQ ID NO: 19 - protein, 13 aa, Apis mellifera
PHDLQALSPPQTS

SEQ ID NO: 20 - protein, 14 aa, Apis mellifera
VMSEKVQSLAGSIY

SEQ ID NO: 21 - protein, 15 aa, Apis mellifera
VAVGDENYGEGSSRE

SEQ ID NO: 22 - protein, 15 aa, Apis mellifera
AREKLALYVYEYLLH

SEQ ID NO: 23 - protein, 15 aa, Apis mellifera
ALSQNPGYLKLRKIR

SEQ ID NO: 24 - protein, 12 aa, Apis mellifera
TPNTILATPFRS

SEQ ID NO: 25 - protein, 15 aa, Apis mellifera
PLKGGLNTPLNNSDF

SEQ ID NO: 26 - protein, 15 aa, Apis mellifera
AQITSQVTGQIGWRR

SEQ ID NO: 27 - protein, 15 aa, Apis mellifera
YIQLKRPSDGATSEP

SEQ ID NO: 28 - protein, 15 aa, Apis mellifera
ATINKRKSFIGTPYW

SEQ ID NO: 29 - protein, 14 aa, Apis mellifera
LSQPGLSQAELSQD

SEQ ID NO: 30 - protein, 15 aa, Apis mellifera
YKERIDEYDYAKPLE

SEQ ID NO: 31 - protein, 15 aa, Apis mellifera
DLDHERMSYLLYQML

SEQ ID NO: 32 - protein, 15 aa, Apis mellifera
KKDNDFIYHERIPDI

SEQ ID NO: 33 - protein, 15 aa, Apis mellifera
ENLDLKLTPYRVLAT

SEQ ID NO: 34 - protein, 14 aa, Apis mellifera
IDHNTRTTQWEDPR

SEQ ID NO: 35 - protein, 15 aa, Apis mellifera
KPLLRRKSDLPQDTY

SEQ ID NO: 36 - protein, 15 aa, Apis mellifera
FGSRSSKTFKPKKNI

SEQ ID NO: 37 - protein, 15 aa, Apis mellifera
FGLARDIYKNDYYRK

SEQ ID NO: 38 - protein, 15 aa, Apis mellifera
KDPTKRPTATELLKH

SEQ ID NO: 39 - protein, 15 aa, Apis mellifera
LREKRRSTGVVHLPS

SEQ ID NO: 40 - protein, 13 aa, Apis mellifera
GSLLTYLRKNTNT

SEQ ID NO: 41 - protein, 15 aa, Apis mellifera
GIANIAISPTIIRKN

SEQ ID NO: 42 - protein, 12 aa, Apis mellifera
ERKKRYTVVGNP

SEQ ID NO: 43 - protein, 14 aa, Apis mellifera
PNDYSSTPGGTLFS

SEQ ID NO: 44 - protein, 15 aa, Apis mellifera
GGTLFSTTPGGTRIV

SEQ ID NO: 45 - protein, 15 aa, Apis mellifera
NLGTIARSGSRAFIE

SEQ ID NO: 46 - protein, 15 aa, Apis mellifera
QSNVAVGTPDYISPE

SEQ ID NO: 47 - protein, 15 aa, Apis mellifera
VRFVVMVTPEDLKAN

SEQ ID NO: 48 - protein, 14 aa, Apis mellifera
ETKMSSSEEVSWIS

SEQ ID NO: 49 - protein, 15 aa, Apis mellifera
SPVIIVGTHYDISYE

SEQ ID NO: 50 - protein, 15 aa, Apis mellifera
GANLSNDYFTNRQDR

SEQ ID NO: 51 - protein, 14 aa, Apis mellifera
FPDEVDTPQDILAK

SEQ ID NO: 52 - protein, 13 aa, Apis mellifera
GGIKGTVIMVPLK

SEQ ID NO: 53 - protein, 15 aa, Apis mellifera
LGLPETETELDNLTE

SEQ ID NO: 54 - protein, 15 aa, Apis mellifera
TENEMTGYVATRWYR

SEQ ID NO: 55 - protein, 14 aa, Apis mellifera
TFLSKHSLSMKFTY

SEQ ID NO: 56 - protein, 15 aa, Apis mellifera
LAREVYKTTRMSAAG

SEQ ID NO: 57 - protein, 14 aa, Apis mellifera
YKTTRMSAAGTYAW

SEQ ID NO: 58 - protein, 15 aa, Apis mellifera
LLLALDGTLKISDFG

SEQ ID NO: 59 - protein, 15 aa, Apis mellifera
NRRALAYSTVGTPDY

SEQ ID NO: 60 - protein, 15 aa, Apis mellifera
DWVFINYTFKRFEGL

SEQ ID NO: 61 - protein, 13 aa, Apis mellifera
EEEIDLSDIDLDE

SEQ ID NO: 62 - protein, 15 aa, Apis mellifera
MGTVLSFSPRDRRGS

SEQ ID NO: 63 - protein, 15 aa, Apis mellifera
YPLPDCDSDEDEDYK

SEQ ID NO: 64 - protein, 14 aa, Apis mellifera
YWGVDELYLESCCQ

SEQ ID NO: 65 - protein, 14 aa, Apis mellifera
EDQQFVSLYGTEEY

SEQ ID NO: 66 - protein, 13 aa, Apis mellifera
GRKTWTFCGTPEY

SEQ ID NO: 67 - protein, 15 aa, Apis mellifera
KIPRGPPSPPAPVMH

SEQ ID NO: 68 - protein, 15 aa, Apis mellifera
SVVHRQETVDCLKKF

SEQ ID NO: 69 - protein, 14 aa, Apis mellifera
GWEKRLSRSTGQHY

SEQ ID NO: 70 - protein, 15 aa, Apis mellifera
SHLLVKHSGSRRPSS

SEQ ID NO: 71 - protein, 15 aa, Apis mellifera
IMRLRKKTLQEDEIA

SEQ ID NO: 72 - protein, 15 aa, Apis mellifera
HAGFLTEYVATRWYR

SEQ ID NO: 73 - protein, 14 aa, Apis mellifera
ENPLRALYLIATNG

SEQ ID NO: 74 - protein, 15 aa, Apis mellifera
QGASGTVYTAIETST

SEQ ID NO: 75 - protein, 14 aa, Apis mellifera
EPKKKKYAKEAWPG

SEQ ID NO: 76 - protein, 15 aa, Apis mellifera
VDPMKRATIEDIKKH

SEQ ID NO: 77 - protein, 14 aa, Apis mellifera
HLAWVNTPRKQGGL

SEQ ID NO: 78 - protein, 15 aa, Apis mellifera
ETRICKIYDSPCLPE

SEQ ID NO: 79 - protein, 14 aa, Apis mellifera
VATRRGSPLLVGIK

SEQ ID NO: 80 - protein, 15 aa, Apis mellifera
RYVLDDQYTSSGGTK

SEQ ID NO: 81 - protein, 15 aa, Apis mellifera
DRTNDKVYDCTTSVV

SEQ ID NO: 82 - protein, 15 aa, Apis mellifera
IVDEEGDYSTPATRD

SEQ ID NO: 83 - protein, 14 aa, Apis mellifera
IQLKRPSDGALSEP

SEQ ID NO: 84 - protein, 15 aa, Apis mellifera
RPSDGDCSEPVKFTY

SEQ ID NO: 85 - protein, 15 aa, Apis mellifera
GANRVVIYDPDWNPA

SEQ ID NO: 86 - protein, 15 aa, Apis mellifera
LAKEKKLYANMFDKF

SEQ ID NO: 87 - protein, 15 aa, Apis mellifera
VSGQLIDSMANSFVG

SEQ ID NO: 88 - protein, 15 aa, Apis mellifera
KICDFGVSGQLIDSM

SEQ ID NO: 89 - protein, 15 aa, Apis mellifera
LLIDEEDYQGLRTSI

SEQ ID NO: 90 - protein, 15 aa, Apis mellifera
EKIGEGTYGTVFKAK

SEQ ID NO: 91 - protein, 11 aa, Apis mellifera
FRPRASSNASS

SEQ ID NO: 92 - protein, 14 aa, Apis mellifera
SLRKLNSRLFVIRG

SEQ ID NO: 93 - protein, 15 aa, Apis mellifera
ATKAARKSAPSTGGV

SEQ ID NO: 94 - protein, 15 aa, Apis mellifera
GYSNEENTWEPEENL

SEQ ID NO: 95 - protein, 15 aa, Apis mellifera
TRMSAAGTYAWMAPE

SEQ ID NO: 96 - protein, 15 aa, Apis mellifera
GSRVRVDSMAKIAEL

SEQ ID NO: 97 - protein, 15 aa, Apis mellifera
HDLRSRLTIIPQDPV

SEQ ID NO: 98 - protein, 15 aa, Apis mellifera
KQTARKSTGGKAPRK

SEQ ID NO: 99 - protein, 15 aa, Apis mellifera
DWGLAEFYHPGQEYN

SEQ ID NO: 100 - protein, 15 aa, Apis mellifera
EMHHEALTEALPGDN

SEQ ID NO: 101 - protein, 13 aa, Apis mellifera
EPHVTRRTPDYFL

SEQ ID NO: 102 - protein, 15 aa, Apis mellifera
LCFSPNRYWLCAAFG

SEQ ID NO: 103 - protein, 15 aa, Apis mellifera
LTLWTSDTQGDADEA

SEQ ID NO: 104 - protein, 15 aa, Apis mellifera
YDRLRPLSYPQTDVF

SEQ ID NO: 105 - protein, 15 aa, Apis mellifera
GQAPGYSYTDANKGK

SEQ ID NO: 106 - protein, 15 aa, Apis mellifera
DRKVKAKSIVFHRKK

SEQ ID NO: 107 - protein, 15 aa, Apis mellifera
RGGVKRISGLIYEET

SEQ ID NO: 108 - protein, 15 aa, Apis mellifera
MARKMKDTDSEEEIR

SEQ ID NO: 109 - protein, 15 aa, Apis mellifera
KELQRQASPSIVIAL

SEQ ID NO: 110 - protein, 15 aa, Apis mellifera
APSSRRNTLCGTLDY

SEQ ID NO: 111 - protein, 12 aa, Apis mellifera
FPLRKTASEPNL

SEQ ID NO: 112 - protein, 15 aa, Apis mellifera
LGENTVRTIAMDGTE

SEQ ID NO: 113 - protein, 15 aa, Apis mellifera
GETRFTDTRKDEQER

SEQ ID NO: 114 - protein, 14 aa, Apis mellifera
LRSRCGTNEDCKNL

SEQ ID NO: 115 - protein, 15 aa, Apis mellifera
AMRFARRSVSDNDIR

SEQ ID NO: 116 - protein, 15 aa, Apis mellifera
SYVDPHTYEDPNQAV

SEQ ID NO: 117 - protein, 14 aa, Apis mellifera
YVDPHTYEDPNQAV

SEQ ID NO: 118 - protein, 15 aa, Apis mellifera
RPTENEMTGYVATRW

SEQ ID NO: 119 - protein, 14 aa, Apis mellifera
ENEMTGYVATRWYR

SEQ ID NO: 120 - protein, 14 aa, Apis mellifera
ERKKRYTVVGNPYW

SEQ ID NO: 121 - protein, 15 aa, Apis mellifera
HEGERKKTVCGTPNY

SEQ ID NO: 122 - protein, 15 aa, Apis mellifera
LELCRKRSMMELHKR

SEQ ID NO: 123 - protein, 13 aa, Apis mellifera
GSLVGTLNYVAPE

SEQ ID NO: 124 - protein, 14 aa, Apis mellifera
GKVARGSYSVSSLF

SEQ ID NO: 125 - protein, 15 aa, Apis mellifera
QVARGMEYLASRRCI

SEQ ID NO: 126 - protein, 15 aa, Apis mellifera
DSEFTCKTPKDSPGV

SEQ ID NO: 127 - protein, 15 aa, Apis mellifera
TCKTPKDSPGVPPSA

SEQ ID NO: 128 - protein, 11 aa, Apis mellifera
KDQAFSKYYTP

SEQ ID NO: 129 - protein, 15 aa, Apis mellifera
RRNTLCGTLDYLPPE

SEQ ID NO: 130 - protein, 15 aa, Apis mellifera
NGQPNRYTNRVVTLW

SEQ ID NO: 131 - protein, 15 aa, Apis mellifera
GSPNRINTHQVVTRW

SEQ ID NO: 132 - protein, 14 aa, Apis mellifera
LPPKSKGTVTYIAP

SEQ ID NO: 133 - protein, 15 aa, Apis mellifera
KGEPNVSYICSRYYR

SEQ ID NO: 134 - protein, 15 aa, Apis mellifera
LDEPNPNSPANSLAA

SEQ ID NO: 135 - protein, 15 aa, Apis mellifera
FSRLGVLSPDCRCKS

SEQ ID NO: 136 - protein, 12 aa, Apis mellifera
DTLQTPCYTPYY

SEQ ID NO: 137 - protein, 15 aa, Apis mellifera
SNHGLAISPGMKKRI

SEQ ID NO: 138 - protein, 13 aa, Apis mellifera
ELDDLMASLSEFK

SEQ ID NO: 139 - protein, 15 aa, Apis mellifera
KISEKKMSTPVEVLC

SEQ ID NO: 140 - protein, 14 aa, Apis mellifera
MNRLIKASSKVEVD

SEQ ID NO: 141 - protein, 15 aa, Apis mellifera
TTFMMTPYVVTRYYR

SEQ ID NO: 142 - protein, 15 aa, Apis mellifera
IKRLHVSASNLQKAW

SEQ ID NO: 143 - protein, 15 aa, Apis mellifera
REGDTTATFCGTPNY

SEQ ID NO: 144 - protein, 15 aa, Apis mellifera
ALKQNKFSNKSDMWS

SEQ ID NO: 145 - protein, 15 aa, Apis mellifera
SATLSMAYAGARFGF

SEQ ID NO: 146 - protein, 15 aa, Apis mellifera
PFHFRAPSKILWKTV

SEQ ID NO: 147 - protein, 15 aa, Apis mellifera
GQHVVSGTPGRVFDM

SEQ ID NO: 148 - protein, 15 aa, Apis mellifera
VIGIDLGTTFSCVAV

SEQ ID NO: 149 - protein, 15 aa, Apis mellifera
GQVGTRRYMAPEVLE

SEQ ID NO: 150 - protein, 16 aa, Apis mellifera
SYRTALTHYLDITSNP

SEQ ID NO: 151 - protein, 15 aa, Apis mellifera
MTTSGSGSGLPLLVQ

SEQ ID NO: 152 - protein, 14 aa, Apis mellifera
PTNFEHTVHVGFDA

SEQ ID NO: 153 - protein, 15 aa, Apis mellifera
GLLERRGSSASLTIE

SEQ ID NO: 154 - protein, 15 aa, Apis mellifera
QEYKKRLSMELTNSL

SEQ ID NO: 155 - protein, 15 aa, Apis mellifera
RNEGVATYAAAVLFR

SEQ ID NO: 156 - protein, 15 aa, Apis mellifera
LDIEQFSTVKGVNLD

SEQ ID NO: 157 - protein, 15 aa, Apis mellifera
MQTVTNMYIVNLAIA

SEQ ID NO: 158 - protein, 15 aa, Apis mellifera
ILHLMCISVDRYWAI

SEQ ID NO: 159 - protein, 13 aa, Apis mellifera
HFPQFSYQESHSA

SEQ ID NO: 160 - protein, 15 aa, Apis mellifera
EVLEDNDYGRAVDWW

SEQ ID NO: 161 - protein, 14 aa, Apis mellifera
SLWKGISTSGKVVW

SEQ ID NO: 162 - protein, 15 aa, Apis mellifera
KIGPLGLSPKKVGDD

SEQ ID NO: 163 - protein, 15 aa, Apis mellifera
RRREYQLTDSAKYYL

SEQ ID NO: 164 - protein, 15 aa, Apis mellifera
SMSDPGTSYRTREEI

SEQ ID NO: 165 - protein, 15 aa, Apis mellifera
NGYGMGTSVDRASAS

SEQ ID NO: 166 - protein, 15 aa, Apis mellifera
TYRYYGHSMSDPGTS

SEQ ID NO: 167 - protein, 15 aa, Apis mellifera
IARAVKFTSKLFGRA

SEQ ID NO: 168 - protein, 15 aa, Apis mellifera
ESATEGAYTTRGGKI

SEQ ID NO: 169 - protein, 15 aa, Apis mellifera
LGVLGSPSPEDLECI

SEQ ID NO: 170 - protein, 14 aa, Apis mellifera
HILGVLGSPSPEDL

SEQ ID NO: 171 - protein, 15 aa, Apis mellifera
VATYESAGIRRFALG

SEQ ID NO: 172 - protein, 13 aa, Apis mellifera
LGQQDRSSSAPNV

SEQ ID NO: 173 - protein, 15 aa, Apis mellifera
APEIMLNSKGYTKSI

SEQ ID NO: 174 - protein, 15 aa, Apis mellifera
FLTEYVATRWYRAPE

SEQ ID NO: 175 - protein, 15 aa, Apis mellifera
SMSDPGTSYRTREEV

SEQ ID NO: 176 - protein, 14 aa, Apis mellifera
GRDKYKTLREIRKG

SEQ ID NO: 177 - protein, 14 aa, Apis mellifera
SFGNKHVTPRTLTS

SEQ ID NO: 178 - protein, 15 aa, Apis mellifera
AVLADVSYLMAMEKS

SEQ ID NO: 179 - protein, 15 aa, Apis mellifera
EKIGEGTYGVVYKAK

SEQ ID NO: 180 - protein, 15 aa, Apis mellifera
SPNYSPTSPTYSPTS

SEQ ID NO: 181 - protein, 15 aa, Apis mellifera
FGIPVRVYTHEVVTL

SEQ ID NO: 182 - protein, 15 aa, Apis mellifera
NRVFQGFTYVAPSIL

SEQ ID NO: 183 - protein, 15 aa, Apis mellifera
QDGTVTHTFCGTIEY

SEQ ID NO: 184 - protein, 15 aa, Apis mellifera
GVPVRTYTHEIVTLW

SEQ ID NO: 185 - protein, 15 aa, Apis mellifera
SGVEVRVTPHRTEII

SEQ ID NO: 186 - protein, 15 aa, Apis mellifera
TAEQYELYCEMGSTF

SEQ ID NO: 187 - protein, 15 aa, Apis mellifera
VKDIEMFSMCEHHLV

SEQ ID NO: 188 - protein, 15 aa, Apis mellifera
NPEGRNVYKDYKQLE

SEQ ID NO: 189 - protein, 15 aa, Apis mellifera
KEFNSYGARRGNDDV

SEQ ID NO: 190 - protein, 13 aa, Apis mellifera
KALRFRYECEGRS

SEQ ID NO: 191 - protein, 15 aa, Apis mellifera
VQIWRRSFDTPPPPM

SEQ ID NO: 192 - protein, 15 aa, Apis mellifera
KGFGYKGSSFHRVIP

SEQ ID NO: 193 - protein, 15 aa, Apis mellifera
RVQGRTWTLCGTPEY

SEQ ID NO: 194 - protein, 15 aa, Apis mellifera
VAMLMQEYTQSGGVR

SEQ ID NO: 195 - protein, 15 aa, Apis mellifera
ADQTPTPTRFIRNCE

SEQ ID NO: 196 - protein, 15 aa, Apis mellifera
ISWYDNEYGYSCRVI

SEQ ID NO: 197 - protein, 15 aa, Apis mellifera
VDRRKAATLRERRRL

SEQ ID NO: 198 - protein, 15 aa, Apis mellifera
FGLATAKTRWSGSQQ

SEQ ID NO: 199 - protein, 15 aa, Apis mellifera
IGDFGLATAKTRWSG

SEQ ID NO: 200 - protein, 15 aa, Apis mellifera
FSHGTHEYHAETIAN

SEQ ID NO: 201 - protein, 15 aa, Apis mellifera
KVMKFSVSPVVRVAV

SEQ ID NO: 202 - protein, 14 aa, Apis mellifera
TYRIGHHSTSDDST

SEQ ID NO: 203 - protein, 15 aa, Apis mellifera
DNKLRRYTTFSKRKT

SEQ ID NO: 204 - protein, 15 aa, Apis mellifera
LRRYTTFSKRKTGIM

SEQ ID NO: 205 - protein, 15 aa, Apis mellifera
YDFEMRLTSVVVTQW

SEQ ID NO: 206 - protein, 15 aa, Apis mellifera
QEEYGEFYKSLTNDW

SEQ ID NO: 207 - protein, 15 aa, Apis mellifera
DLTYGSRYKDLKLPD

SEQ ID NO: 208 - protein, 15 aa, Apis mellifera
QEKRKQISVRGIVDV

SEQ ID NO: 209 - protein, 15 aa, Apis mellifera
VFDLGGGTFDVSLLT

SEQ ID NO: 210 - protein, 15 aa, Apis mellifera
EKITRRFTLELAKKG

SEQ ID NO: 211 - protein, 15 aa, Apis mellifera
ILEQSWGSPKITKDG

SEQ ID NO: 212 - protein, 15 aa, Apis mellifera
QTAQGMDYLHAKNII

SEQ ID NO: 213 - protein, 15 aa, Apis mellifera
DPTIEDSYTKQCVID

SEQ ID NO: 214 - protein, 15 aa, Apis mellifera
DVHRTLKYGGIFLYP

SEQ ID NO: 215 - protein, 14 aa, Apis mellifera
TFCGTPDYIAPEII

SEQ ID NO: 216 - protein, 15 aa, Apis mellifera
MSDPGTSYRTREEIQ

SEQ ID NO: 217 - protein, 15 aa, Apis mellifera
NNGYGMGTSVDRASA

SEQ ID NO: 218 - protein, 15 aa, Apis mellifera
LEMVTYRYYGHSMSD

SEQ ID NO: 219 - protein, 15 aa, Apis mellifera
KAKAGTGSATLSMAY

SEQ ID NO: 220 - protein, 15 aa, Apis mellifera
KENQKHIYYITGESR

SEQ ID NO: 221 - protein, 15 aa, Apis mellifera
QGNRTTPSYVAFTDT

SEQ ID NO: 222 - protein, 14 aa, Apis mellifera
KVYGKTSHLRAHLR

SEQ ID NO: 223 - protein, 15 aa, Apis mellifera
ESLADHVYTSKSDVW

SEQ ID NO: 224 - protein, 15 aa, Apis mellifera
DVYEDDAYLKRSKGR

SEQ ID NO: 225 - protein, 15 aa, Apis mellifera
NKNDRTLTILDSGIG

SEQ ID NO: 226 - protein, 12 aa, Apis mellifera
SIFWCNLSPNGG

SEQ ID NO: 227 - protein, 15 aa, Apis mellifera
GPRVHFVSNIDGTHI

SEQ ID NO: 228 - protein, 15 aa, Apis mellifera
QLDEILRYHTEIVFA

SEQ ID NO: 229 - protein, 15 aa, Apis mellifera
TSKVALVYGQMNEPP

SEQ ID NO: 230 - protein, 15 aa, Apis mellifera
MKKIRLESDDEGIPS

SEQ ID NO: 231 - protein, 15 aa, Apis mellifera
KTVNKDATDRERIEF

SEQ ID NO: 232 - protein, 15 aa, Apis mellifera
QTEFMGFSFLNPEFV

SEQ ID NO: 233 - protein, 15 aa, Apis mellifera
LNMLKLSSPELEKFI

SEQ ID NO: 234 - protein, 13 aa, Apis mellifera
GRTTKTFCGTPEY

SEQ ID NO: 235 - protein, 14 aa, Apis mellifera
ICKTRRNSLFRQGM

SEQ ID NO: 236 - protein, 15 aa, Apis mellifera
IVEGLMTTVHAVTAT

SEQ ID NO: 237 - protein, 14 aa, Apis mellifera
GAFGNVYKGVWVPE

SEQ ID NO: 238 - protein, 15 aa, Apis mellifera
QNIIPAATGAAKAVG

SEQ ID NO: 239 - protein, 15 aa, Apis mellifera
GILAADESTATIGKR

SEQ ID NO: 240 - protein, 15 aa, Apis mellifera
IIHRDLKSNNIFLHD

SEQ ID NO: 241 - protein, 15 aa, Apis mellifera
NAKDEIVYCKFHYKT

SEQ ID NO: 242 - protein, 15 aa, Apis mellifera
YFAKALENPERPFLA

SEQ ID NO: 243 - protein, 15 aa, Apis mellifera
RLMRDDTYTAHAGAK

SEQ ID NO: 244 - protein, 15 aa, Apis mellifera
HKLGGGQYGDVYEAV

SEQ ID NO: 245 - protein, 15 aa, Apis mellifera
DNTNGCTSAGAHFNP

SEQ ID NO: 246 - protein, 14 aa, Apis mellifera
IKLKGYTSWAIGLS

SEQ ID NO: 247 - protein, 15 aa, Apis mellifera
KKVIGSAYEVIKLKG

SEQ ID NO: 248 - protein, 15 aa, Apis mellifera
MMKTFCGTPMYVAPE

SEQ ID NO: 249 - protein, 15 aa, Apis mellifera
VSAQLDRTIGRRNTF

SEQ ID NO: 250 - protein, 15 aa, Apis mellifera
SRQKVRHTFVGTPCW

SEQ ID NO: 251 - protein, 15 aa, Apis mellifera
GLCPSTETFTGTLQY

SEQ ID NO: 252 - protein, 12 aa, Apis mellifera
NYTEYVATRWYR

SEQ ID NO: 253 - protein, 15 aa, Apis mellifera
QNAMIPQYQMLFSMD

SEQ ID NO: 254 - protein, 14 aa, Apis mellifera
NVTRSDYLETFEFI

SEQ ID NO: 255 - protein, 14 aa, Apis mellifera
TEYGSGYPNDPETK

SEQ ID NO: 256 - protein, 15 aa, Apis mellifera
AAERCSMPYRAPELF

SEQ ID NO: 257 - protein, 15 aa, Apis mellifera
DKIFRGYSYVAPSIL

SEQ ID NO: 258 - protein, 14 aa, Apis mellifera
PARKLTATPTPIAG

SEQ ID NO: 259 - protein, 15 aa, Apis mellifera
GYHYNALTCEGCKGF

SEQ ID NO: 260 - protein, 15 aa, Apis mellifera
KLQLGRVSFSRRSGV

SEQ ID NO: 261 - protein, 15 aa, Apis mellifera
IIVAGTLTNKMAPAL

SEQ ID NO: 262 - protein, 15 aa, Apis mellifera
RYPRGESYEDLVARL

SEQ ID NO: 263 - protein, 14 aa, Apis mellifera
VKVNPKYFRPTEVD

SEQ ID NO: 264 - protein, 14 aa, Apis mellifera
EQRLLNTFCGSPLY

SEQ ID NO: 265 - protein, 15 aa, Apis mellifera
NLAAEKTYNNLDVSL

SEQ ID NO: 266 - protein, 14 aa, Apis mellifera
DQNKHMTQEVVTQY

SEQ ID NO: 267 - protein, 15 aa, Apis mellifera
DTGENPIYKQATSTF

SEQ ID NO: 268 - protein, 14 aa, Apis mellifera
DPTLTDYVATRWYR

SEQ ID NO: 269 - protein, 13 aa, Apis mellifera
TKDLGGQSSTTEF

SEQ ID NO: 270 - protein, 15 aa, Apis mellifera
VYKVIVGSPAHLGGL

SEQ ID NO: 271 - protein, 15 aa, Apis mellifera
CDLNTYMTNNKGSAA

SEQ ID NO: 272 - protein, 15 aa, Apis mellifera
YMTNNKGSAAWMAPE

SEQ ID NO: 273 - protein, 15 aa, Apis mellifera
VEGQDMLYQSLKLTN

SEQ ID NO: 274 - protein, 14 aa, Apis mellifera
TETFTGTLQYMAPE

SEQ ID NO: 275 - protein, 13 aa, Apis mellifera
AIQCRVTTEDPAK

SEQ ID NO: 276 - protein, 12 aa, Apis mellifera
EMTGYVATRWYR

SEQ ID NO: 277 - protein, 15 aa, Apis mellifera
ELLWKQTYSCSVDYW

SEQ ID NO: 278 - protein, 15 aa, Apis mellifera
TFIGTLEYLAPEIIQ

SEQ ID NO: 279 - protein, 15 aa, Apis mellifera
LVDSKAKTRSAGCAA

SEQ ID NO: 280 - protein, 15 aa, Apis mellifera
MSYGERPYWNWSNQD

SEQ ID NO: 281 - protein, 15 aa, Apis mellifera
VATPQLLTPVGSADF

SEQ ID NO: 282 - protein, 15 aa, Apis mellifera
TVWSAPNYCYRCGNV

SEQ ID NO: 283 - protein, 15 aa, Apis mellifera
GCHLLVATPGRLVDM

SEQ ID NO: 284 - protein, 14 aa, Apis mellifera
PDEKHLTMCGTPNY

SEQ ID NO: 285 - protein, 15 aa, Apis mellifera
QTAPRAGTPGFRAPE

SEQ ID NO: 286 - protein, 15 aa, Apis mellifera
TPMHGSQTPMYENGS

SEQ ID NO: 287 - protein, 15 aa, Apis mellifera
LYDGYIVYSERDEDF

SEQ ID NO: 288 - protein, 14 aa, Apis mellifera
EPATINYPFEKGPL

(19) United States
(12) Patent Application Publication
(10) Pub. No.: US 2015/0153354 A1
(43) Pub. Date: Jun. 4, 2015

(54) Methods and Compositions for Characterizing Phenotypes Using Kinome Analysis

(71) Applicants: Brett Trost, Saskatoon (CA); Anthony Kusalik, Saskatoon (CA); Scott Napper, Saskatoon (CA); Albert Robertson, Saskatoon (CA); Philip Griebel, Saskatoon (CA)

(72) Inventors: Brett Trost, Saskatoon (CA); Anthony Kusalik, Saskatoon (CA); Scott Napper, Saskatoon (CA); Albert Robertson, Saskatoon (CA); Philip Griebel, Saskatoon (CA)

(21) Appl. No.: 14/346,378

(22) PCT Filed: Jun. 24, 2012
(86) PCT No.: PCT/IB2012/001254

§ 371 Date: Feb. 12, 2015

Related U.S. Application Data
(60) Provisional application No. 61/537,941, filed on Sep. 22, 2011.
(60) Provisional application No. 61/619,902, filed on Apr. 3, 2012.

(51) Int. Cl. G01N 33/68; C12Q 1/48

(52) U.S. Cl. CPC G01N 33/6845; C12Q 1/485; G01N 2333/91205; G01N 2440/14

(57) ABSTRACT

Isolated peptides, arrays comprising a plurality of peptides and methods of use thereof are provided which can be used for identifying bee phenotypes and selecting bee lines with favourable characteristics.

### FIELD OF THE DISCLOSURE

Disclosed herein are methods, isolated peptides, arrays and compositions, which can be used for identifying bee phenotypes and selecting bee lines with favourable characteristics.

### INTRODUCTION

Varroa infestation in Apis mellifera is a serious worldwide problem, threatening the existence of the domesticated honey bee and is part of the cause of colony collapse disorder (CCD). Most breeding and research programs have focused on selecting for hygienic behavior, a trait correlated with varroa tolerance.

Tools and methods to aid in breeding gentle and/or productive honey bees with tolerance to mites and/or brood disease would be helpful.

### SUMMARY OF THE DISCLOSURE

An aspect of the disclosure includes a plurality of peptides, each which comprises a sequence of about 5 to about 100 amino acids, for example about 5 to about 50 amino acids or about 5 to about 30 amino acids, wherein the sequence comprises a contiguous sequence present in a peptide sequence selected from the group of SEQ ID NOs: 1 to 288, wherein the contiguous sequence comprises a bee phosphorylation site sequence.

In an embodiment, the plurality of peptides comprises about 5, 10, 15, 20, 25, 50, 75, 100, 125, 150, 175, 200, 225, 250, 275 or 288 peptides each comprising a peptide sequence selected from the group listed in Table 1. In another embodiment, the plurality of peptides comprises about 5, 10, 15, 20, 25, 50, 75, 100, 125, 150, 175, 200, 225, 250, 275 or 288 of peptide sequences listed in Tables 1, 2, 3, and/or 4.

A further aspect includes an array comprising a support and i) a plurality of peptides described herein and/or ii) a plurality of bee species peptides, each peptide comprising a sequence of about 5 to about 50 amino acids, about 5 to about 30 amino acids or about 8 to about 15 amino acids, wherein the sequence comprises a phosphorylation site sequence.

In an embodiment, each of the array plurality of peptides comprises a sequence that is about 8 to about 15 amino acids of a peptide sequence selected from SEQ ID NO: 1-288.

In another embodiment, the array described herein comprises a plurality of peptides each peptide comprising a peptide sequence selected from the group listed in Table 2, 3, and/or 4.

In an embodiment, each peptide is spotted on the support in duplicate, triplicate or more.

In yet another embodiment, the array plurality of peptides comprises at least 25, 50, 75, 100, 125, 150, 200, 250, 275, 288 or at least about 300 different peptides.

Also provided is a method for measuring protein kinase activity in a sample from a subject using for example a plurality of peptides and/or an array described herein, said method comprising the steps of:
a. obtaining the sample from the subject;
b. incubating said sample with ATP or other suitable ATP analog and a plurality of peptides described herein; and
c. determining a detectable phosphorylation profile, said phosphorylation profile resulting from the interaction of the sample with the plurality of peptides;
wherein the detectable phosphorylation profile provides a measure of the protein kinase activity in the sample

In an embodiment the method for measuring protein kinase activity in a sample from a subject (e.g. a bee), comprises the steps of: a) obtaining a sample of the subject; b) incubating said sample with ATP and/or other suitable ATP source and an array of peptides, the array of peptides comprising a plurality of peptides selected from Table 1; and, c) obtaining a detectable phosphorylation profile, said phosphorylation profile resulting from the interaction of the sample with the array of peptides.

In an embodiment, the plurality of peptides is comprised in a composition described herein or on an array described herein.

A further aspect includes a method for identifying a biomarker and/or set of biomarkers in a subject associated with a desirable phenotype, the method comprising:
a. obtaining a sample from the subject;
b. contacting the sample with ATP or other suitable ATP analog and a plurality of peptides described herein, optionally comprised in an composition and/or on an array;
c. determining a phosphorylation profile of the plurality of peptides;
d. comparing the phosphorylation profile of the plurality of peptides with a control;
wherein a difference or a similarity in the phosphorylation profile of the plurality of peptides between the sample and the control is used to identify the biomarker and/or set of biomarkers associated with the desirable phenotype.

In yet another embodiment, the subject is subjected to a stressor prior to obtaining the sample and/or before obtaining the subject phosphorylation profile in a method described herein.

In an embodiment, the stressor is a pathogen challenge.

In certain embodiments, the method further comprises selecting the subject (or related subjects) comprising the biomarker or set of biomarkers associated with the desirable phenotype. For example, related subjects when referring to bees can be from a same hive, colony or group.

Yet a further aspect includes a method of classifying a subject, the method comprising a) determining a detectable phosphorylation profile of a sample obtained from the subject, said phosphorylation profile resulting from the interaction of the sample with the plurality of peptides described herein (for example comprised in a composition and/or on an array); b) comparing said phosphorylation profile to a reference phosphorylation profile of a known phenotype (e.g. a phenotype reference phosphorylation profile); wherein a difference or a similarity in the phosphorylation profile of the plurality of peptides between the sample and the reference phosphorylation profile is used to classify the subject for example as having or not having a phenotype.

The phosphorylation reference profile can be determined and or predetermined and is for example generated from control subjects with known phenotypes.

In yet another embodiment, a method of classifying a subject comprises: a) determining a detectable phosphorylation profile of a sample obtained from the subject, said phosphorylation profile resulting from the interaction of said sample with the plurality of peptides described herein (for example in a composition or on an array); b) comparing said phosphorylation profile to one or more reference phosphorylation profiles, each reference phosphorylation profile corresponding to a known phenotype (e.g. a phenotype reference phosphorylation profile); and c) classifying the subject according to the probability of said phosphorylation profile falling within a class defined by said reference phosphorylation profile.

A further aspect includes a method of screening a subject for susceptibility and/or resistance to a pathogen, the method comprising:
a. obtaining a sample from the subject;
b. contacting the sample with ATP and/or a suitable ATP analog and the plurality of peptides described herein (for example in a composition and/or on an array);
c. determining a phosphorylation profile of the plurality of peptides;
d. comparing the phosphorylation profile of the plurality of peptides with one or more reference phosphorylation profiles;
wherein a difference or a similarity in the phosphorylation profile of the plurality of peptides between the sample and the one or more reference phosphorylation profiles identifies the subject as susceptible or resistant to the pathogen.

Also provided in a further aspect is a method of aiding selection of a subject (or related subjects) with a desirable phenotype comprising:
a. determining a subject phosphorylation profile from a sample obtained from the subject;
b. providing one or more reference phosphorylation profiles associated with a known phenotype, wherein the subject phosphorylation profile and the reference phosphorylation profile(s) have one or a plurality of values, each value representing a phosphorylation level of a peptide selected from the plurality of peptides described herein;
c. identifying the reference phosphorylation profile most similar to the subject phosphorylation profile,
wherein the subject is predicted to have the phenotype of the reference phosphorylation profile most similar to the subject phosphorylation profile.

In certain embodiments, the methods described herein further comprise obtaining a sample from the subject. The sample can for example be the subject (e.g. a bee) or a part thereof (e.g. a thorax).

In an embodiment, the methods described herein are used for screening for varroa resistance.

In certain embodiments, for example, wherein the subject is infected with varroa, decreased phosphorylation, relative to an uninfected subject, of two or more peptides corresponding to peptides in Table 2A and/or 3A (e.g. each peptide may have more or less sequence than provided in the table), is indicative that the subject is varroa resistant and/or increased phosphorylation, relative to an uninfected subject, of two or more peptides in Table 2B and/or 3B is indicative that the subject is varroa resistant.

In other embodiments, wherein the subject is uninfected with varroa, decreased phosphorylation, relative to a varroa-sensitive subject, of two or more peptides corresponding to peptides in Table 2A and/or 4A (e.g. each peptide may have more or less sequence than provided in the Table) is indicative that the subject is varroa resistant and/or increased phosphorylation of two or more peptides in Table 26 and/or 4B, relative to a varroa-sensitive subject, is indicative that the subject is varroa resistant.

In an embodiment, the method comprises assessing for Nosema resistance, for example the method can comprise measuring protein kinase activity in a sample from a subject suspected of having Nosema resistance, identifying a biomarker associated with Nosema resistance, classifying a subject to determine if the subject has Nosema resistance, aiding in selecting subjects with Nosema resistance and screening for Nosema resistance. Any other phenotype can further be assessed similarly by the methods described herein.

In an embodiment, the subject is a bee, such as a honey bee.

A method for phenotyping a subject, the method comprising a) obtaining a sample of the subject; b) incubating said sample with ATP and/or a suitable ATP analog and the plurality of peptides described herein for example comprised in a composition or on an array, each peptide comprising a phosphorylation site sequence; and c) determining a detectable phosphorylation profile, said phosphorylation profile resulting from the interaction of the sample with the plurality of peptides; d) comparing said phosphorylation profile to a reference phosphorylation profile of a known phenotype; wherein a difference or a similarity in the phosphorylation profile of the plurality of peptides between the sample and the reference phosphorylation profile is used to classify the subject as having or not having the phenotype.

In an embodiment, the subject is identified as having the phenotype associated with a reference phosphorylation profile if the subject phosphorylation profile is similar to said reference phosphorylation profile.

In certain embodiments, the method further comprises e) identifying the subject as having the phenotype associated with the reference phosphorylation profile if said phosphorylation profile is similar to the reference phosphorylation profile or identifying the subject as not having the phenotype associated with the reference phosphorylation profile if the said phosphorylation profile is not similar to the reference phosphorylation profile.

In various embodiments, the subject phosphorylation profile is compared to one or more reference phosphorylation profiles, wherein the subject is identified as having or likely having the phenotype of the reference phosphorylation profile most similar to said subject phosphorylation profile.

In methods described herein, the step of determining a phosphorylation profile can comprise:
a. obtaining one or more datasets, each dataset comprising a phosphorylation signal intensity for each peptide of the plurality of peptides;
b. transforming the phosphorylation signal intensity of each peptide of the plurality of peptides using a variance stabilizing transformation to provide a variance stabilized signal intensity for each peptide of the plurality of peptides; and
c. identifying one or more peptides of the plurality of peptides that are consistently phosphorylated or consistently unphosphorylated,
thereby providing a subject phosphorylation profile.

In an embodiment, the step of obtaining a detectable phosphorylation profile comprises:
a) obtaining one or more datasets, each dataset comprising a phosphorylation signal intensity for each peptide of the plurality of peptides;
b) transforming the phosphorylation signal intensity of each peptide of the plurality of peptides using a variance stabilizing transformation to provide a variance stabilized signal intensity for each peptide of the plurality of peptides; and
c) identifying one or more peptides of the plurality of peptides that are consistently phosphorylated or consistently unphosphorylated,
   thereby providing a bee phosphorylation profile.

In an embodiment, each peptide of the plurality is present in at least two replicates, and the method of obtaining the detectable phosphorylation profile comprises:
a) obtaining one or more datasets, each dataset comprising a phosphorylation signal intensity for each replicate of the plurality of peptides;
b) transforming the phosphorylation signal intensity of each replicate of the plurality of peptides using a variance stabilizing transformation to provide a variance stabilized signal intensity for each replicate of the plurality of peptides;
c) identifying one or more peptides of the plurality of peptides that are consistently phosphorylated or consistently unphosphorylated by calculating a phosphorylation consistency value for each peptide of the plurality of peptides, calculating the phosphorylation consistency value optionally comprising calculating a replicate variability for each peptide using the variance stabilized signal intensity of each replicate of the at least two replicates; and
d) determining a phosphorylation characteristic for a plurality one of the one or more peptides that are consistently phosphorylated or consistently unphosphorylated;

thereby providing a phosphorylation profile.

In an embodiment, the phosphorylation consistency value is calculated using a chi-square (χ²) test.

In another embodiment, the method further comprises outputting a phosphorylation characteristic of the one or more peptides of the plurality of peptides.

In an embodiment, the phosphorylation characteristic is differential phosphorylation compared to a control.

Another aspect includes a phosphorylation profile obtained using a method described herein.

In an embodiment, the phosphorylation profile is presented in pseudo-images generated for example based on the p-values from the one-sided t-tests for phosphorylation or dephosphorylation of each peptide. Each peptide is optionally represented by one small colored circle, wherein the depths of the coloration are inversely related to the corresponding p-values.

In a further aspect the disclosure includes a kit comprising a plurality of peptides described herein, an array described herein, and/or a kit control and/or package housing the peptides, array and/or kit control.

Other features and advantages of the present disclosure will become apparent from the following detailed description. It should be understood, however, that the detailed description and the specific examples while indicating preferred embodiments of the disclosure are given by way of illustration only, since various changes and modifications within the spirit and scope of the disclosure will become apparent to those skilled in the art from this detailed description.

### BRIEF DESCRIPTION OF THE DRAWINGS

An embodiment of the disclosure will now be discussed in relation to the drawings in which:

FIG. 1. Comparison of varroa population growth in G4, a varroa sensitive colony, and S88 a varroa tolerant colony. Percent adult varroa infestations rapidly increased in G4 from 2 to 67% in 88 days, whereas varroa infestations in the tolerant colony remained below 5% (FIG. 2).

FIG. 2. The varroa tolerant colony is S88 and the varroa sensitive colony is G4. The varroa sensitive line G4 collapsed and died 17 months from construction, whereas the varroa tolerant colony survived 52 months before death. Varroa infestation levels in S88 never exceeded 18%. Standard errors are sample means (n=5) of percent adult bee varroa infestations. Adult bee varroa infestations were determined by alcohol washes.

FIG. 3. Clustering and Heat Map of Kinome Data

FIG. 4. Heat Map of Validation using Bee Heads and Thorax.

FIG. 5: A general workflow of the kinome analysis. The flow chart starts from the top left and follows the directions by the arrows. The rectangles represent procedures, and the oval, the intermediate result.

### DETAILED DESCRIPTION OF THE DISCLOSURE

A bee peptide array for assessing bee and related species phosphorylation profiles is provided in an aspect of the disclosure. It is demonstrated herein using said array that bees that are tolerant to varroa infection (S88) have a different phosphorylation profile compared to bees that are sensitive to varroa infection (G4). Differences are visible in uninfected bees as well as infected bees. The phosphorylation profiles can be used to classify bees as tolerant or sensitive to varroa infection. Similarly the arrays can be used to obtain phosphorylation profiles for classifying bees for other characteristics.

In an aspect, the disclosure includes an isolated peptide which comprises a sequence of about 5 to about 100 amino acids, for example about 5 to about 50 amino acids or about 5 to about 30 amino acids, wherein the sequence comprises a contiguous sequence present in a peptide sequence selected from the group of SEQ ID NOs: 1 to 288, said contiguous sequence comprising a bee phosphorylation site sequence. For example, each of the sequences in Table 1 (SEQ ID NOs: 1-288) comprise a bee phosphorylation site sequence. The isolated peptide for example comprises minimally the portion of a sequence in Table 1 that comprises said phosphorylation site sequence.

In another aspect, the disclosure includes a plurality of peptides (e.g. a collection), each comprising a sequence of about 5 to about 100 amino acids, for example about 5 to about 50 amino acids or about 5 to about 30 amino acids, wherein the sequence comprises a contiguous sequence present in an amino acid sequence selected from the group of SEQ ID NOs: 1 to 288, said contiguous sequence comprising a bee phosphorylation site sequence.

In an embodiment, the plurality of peptides comprises a subset (e.g. two or more) of the peptides or parts thereof (the parts comprising a bee phosphorylation site sequence) listed in Table 1, for example, about 5, 10, 15, 20, 25, 50, 75, 100, 125, 150, 175, 200, 225, 250, 275 or 288 of the peptides listed in Table 1. In an embodiment, the plurality of peptides comprises a subset (e.g. 2 or more) of the peptides listed in Table 2, 3 and/or 4. In a further embodiment, the plurality of peptides comprises the set of peptides in Tables 1, 2, 3 or 4.

Each of the plurality of peptides is for example an isolated peptide, for example an isolated synthetic chemically peptide synthesized using for example commercially available methods and equipment.

In another aspect, the disclosure includes an array comprising a plurality of peptides. In an embodiment, each peptide comprises an amino acid sequence of about 5 to about 100 amino acids, for example about 5 to about 50 amino acids or about 5 to about 30 amino acids, and comprises a bee phosphorylation site sequence, each peptide comprising at least one serine, threonine or tyrosine amino acid residue. In another embodiment, the array comprises a plurality of peptides, each comprising an amino acid sequence of about 5 to about 100 amino acids, for example about 5 to about 50 amino acids or about 5 to about 30 amino acids, wherein the sequence comprises a contiguous sequence present in an amino acid sequence selected from the group of SEQ ID NOs: 1 to 288, said contiguous sequence comprising a bee phosphorylation site sequence.

The peptide sequences can be selected for example using the method described below in Example 4.

In an embodiment, the array is a bee specific array. In another embodiment, the plurality of peptides (e.g also referred to as peptide targets) is attached to a support surface, each peptide comprising a sequence of a bee phosphorylation site sequence selected for example according to a method described herein, such as in Example 4, wherein the similarity is below a preselected threshold.

The term “phosphorylation site sequence” means a peptide sequence consisting of at least 5 residues and less than 30 residues and/or 30 or fewer residues (for example 15 residues) and that comprises at least one serine, threonine or tyrosine residue phosphorylatable or predicted to be phosphorylatable by one or more kinases.

The plurality of peptides and/or array comprising a plurality of peptides such as the peptides described in Table 1, can be used for example for bee phenotyping by kinome analysis. As demonstrated below, an array comprising a plurality of bee peptide sequences can be used to distinguish one bee phenotype (e.g. verroa resistance) from another (e.g. verroa tolerance).

In an aspect, the disclosure includes an array comprising a plurality of peptides selected from the peptides, and/or parts of said peptides comprising a bee phosphorylation site sequence, listed in Table 1. Subsets of peptides are listed in Table 2, 3, and 4. In an embodiment, the plurality of peptides comprises the peptides (or parts of said peptides comprising a bee phosphorylation site sequence) listed in Table 1 and/or the peptides (or parts of said peptides comprising a bee phosphorylation site sequence) listed in Table 2, 3 or 4.

Each of the peptides in Table 1 comprises a bee phosphorylation site sequence, optionally a predicted bee phosphorylation site sequence and/or a known or confirmed bee phosphorylation site sequence.

Each of the peptides comprising sequences selected from Table 1, can for example, comprise 5, 6, 7, 8, 9, 10, 11, 12, 13, 14, 15, 16, 17, 18, 19, 20, 21, 22, 23, 24, 25, 26, 27, 28, 29 or 30 or more amino acids. For example, if SEQ ID NO:1 is selected, the peptide can comprise 8, 9, 10, 11, 12, 13, 14 or 15 of SEQ ID NO:1 as long as the phosphorylation site is included. Preferably, the phosphorylation site is centered or about centered in the peptide length selected. Typical phosphorylatable amino acids include serine, threonine and tyrosine residues.

Longer sequences comprising the sequence of a SEQ ID NO: and surrounding sequence (e.g. sequence found in the naturally occurring protein for example according to the provided accession number in Table 1) can also be used. For example, the sequence can be 16-200 amino acids, 16-100 amino acids, or 16 to 50 amino acids. The peptide can also be the full length polypeptide (e.g. the full length protein).

The peptides can also for example comprise linkers (e.g. flexible linkers) or other sequence not present in the surrounding sequence.

Further, each peptide can be spotted on the array singly, in duplicate, in triplicate or greater. For example, the peptide can be spotted, 4, 5, 6, 7, 8 or 9 times or more.

The sequence of the peptide is selected for example as described further in the Examples. For example, the peptide sequences are bee peptide sequences comprising known and/or putative phosphorylation sites, which can be identified by a method such as a computerized method comprising comparing known phosphorylation sites in known proteins in a characterized proteome to the bee proteome and selecting corresponding bee sequences that meet specified criteria. Peptide sequences can also for example be selected by manual inspection of a phosphoproteome database of bees or closely related species.

The term “array” as used herein refers to a two-dimensional arrangement of a plurality of peptide molecules, each peptide comprising a known or putative phosphorylation site, attached on a support surface such as a slide or a bead. Arrays are generally comprised of regular, ordered peptide molecules, as in for example, a rectilinear grid, parallel stripes, spirals, and the like, but non-ordered arrays may be advantageously used as well. The arrays generally comprise in the range of about 2 to about 3000 different peptides, more typically about 2 to about 1,200 different peptides. The array can for example comprise 25, 50, 100, 150, 200, 250, 300, 400, 500, 1000, 1200 or more different peptides, spotted in a single replicate, or in replicates of 2, 3, 4, 5, 6, 7, 8, or 9 or greater. For example, depending on the dataset to be obtained, the peptide array can comprise peptides with known phosphorylation motifs (e.g., phosphorylation site sequences), optionally phosphorylation motifs for proteins that are found in a signaling pathway or related pathways. Such peptide arrays can be useful for deciphering peptides phosphorylated or signaling pathways activated by a stressor such as an infectious agent or a macromolecule. The peptide molecules comprise for examples peptides or parts thereof, selected from the peptides listed in Tables 1, 2, 3 or 4.

For example, depending on the dataset to be obtained, the peptide array can comprise peptides with known phosphorylation motifs, optionally phosphorylation motifs for proteins that are found in a signaling pathway or related pathways. Such peptide arrays can be useful for deciphering peptides phosphorylated or signaling pathways activated by a stressor such as an infectious agent or a macromolecule. Alternatively, the peptide array can comprise random peptide sequences comprising putative phosphorylation sites wherein the plurality of peptides or a subset thereof comprises at least one of a serine, threonine or tyrosine residue.

The term “attached,” as in, for example, a support surface having a peptide molecule “attached” thereto, includes covalent binding, adsorption, and physical immobilization. The terms “binding” and “bound” are identical in meaning to the term “attached.” The peptide can for example be attached via a flexible linker.

The term “peptide molecule” or “peptide” as used herein includes a molecule comprising a chain of 5 or more amino acids comprising a known or putative phosphorylation site. A peptide in the context of a peptide array typically comprises a peptide having from about 5 to about 21 amino acid residues or any number in between. The peptide can also be longer, for example up to 30 amino acids, up to 50 amino acids or up to 100 amino acids. For example, the peptide can comprise a sequence listed in Table 1 and additional surrounding cognate protein sequence which can be identified according to the accession number provided in Table 1. An amino acid linker can also be included. A polypeptide and/or protein can comprise any length of amino acid residues.

Generally, since the peptide molecules are typically pre-formed and spotted onto the support as intact molecules, they are comprised of 5 or more amino acids, and are peptides, polypeptides or proteins. For the most part, the peptide molecules in the present arrays comprise about 5 to 100 amino acids, for example 5 to 50 amino acids, preferably about 5 to 30 amino acids. A phosphorylation motif comprises for example 4 amino acids. The amino acids forming all or a part of a peptide molecule may be any of the twenty conventional, naturally occurring amino acids, i.e., alanine (A), cysteine (C), aspartic acid (D), glutamic acid (E), phenylalanine (F), glycine (G), histidine (H), isoleucine (I), lysine (K), leucine (L), methionine (M), asparagine (N), proline (P), glutamine (Q), arginine (R), serine (S), threonine (T), valine (V), tryptophan (W), and tyrosine (Y).

Each peptide corresponds to a protein which can be identified for example by an accession number.

The term “accession number” as used herein refers to a code such as a Genbank accession number that uniquely identifies a particular polypeptide sequence (e.g. protein or part thereof) and/or DNA encoding said polypeptide or part thereof.

The term “corresponds to” as used herein means in the context of a sequence and a second sequence from the same species, sequences that derive from the same (e.g. cognate) protein e.g. a phosphorylation site sequence and a full length polypeptide which contains the phosphorylation site sequence. Similarly, regarding a first sequence and a “corresponding protein identifier” from the same species refers to a protein identifier such as an accession number that identifies the same protein as contains the first sequence.

As used herein, the term “plurality of peptides” means at least 2, for example at least 3 peptides, at least 4 peptides, at least 5 peptides, at least 10, at least 15, at least 25 peptides, at least 50 peptides, at least 100 peptides, at least 200 peptides, at least 300 peptides, at least 400, at least 500 or at least 1000 or any number in between.

In an embodiment, the peptide array comprises at least 2 peptides, at least 3 peptides, at least 4 peptides, at least 5 peptides, at least 25 peptides, at least 50 peptides, at least 100 peptides, at least 200 peptides, at least 300 peptides, at least 400, at least 500 or at least 1000 or any number in between 2 and 1000. Each peptide is optionally spotted in at least two replicates, or at least 3 replicates per array, optionally as replicate blocks. The peptides can be spotted in at least 4, 5, 6, 7, 8 or 9 or up to 15 replicates. For example, the peptides can be either random sequences (e.g. control peptide), not necessarily always containing a Ser/Thr or Tyr, or represent known or predicted phosphorylation sites (for example peptides comprising Ser/Thr or Tyr residues).

Any of the non-phosphorylation site amino acids in the peptide molecules may be replaced by a non-conventional amino acid. In general, conservative replacements are preferred. Conservative replacements substitute the original amino acid with a non-conventional amino acid that resembles the original in one or more of its characteristic properties (e.g., charge, hydrophobicity, stearic bulk; for example, one may replace Val with Nval). The term “non-conventional amino acid” refers to amino acids other than conventional amino acids, and include, for example, isomers and modifications of the conventional amino acids, e.g., D-amino acids, non-protein amino acids, post-translationally modified amino acids, enzymatically modified amino acids, constructs or structures designed to mimic amino acids (e.g., .alpha,.alpha.-disubstituted amino acids, N-alkyl amino acids, lactic acid, .beta.-alanine, naphthylalanine, 3-pyridylalanine, 4-hydroxyproline, 0-phosphoserine, N-acetylserine, N-formylmethionine, 3-methylhistidine, 5-hydroxylysine, and nor-leucine). The peptidic molecules may also contain nonpeptidic backbone linkages, wherein the naturally occurring amide —CONH— linkage is replaced at one or more sites within the peptide backbone with a non-conventional linkage such as N-substituted amide, ester, thioamide, retropeptide (—NHCO—), retrothioamide (—NHCS—), sulfonamido (—SO.sub.2NH—), and/or peptoid (N-substituted glycine) linkages. Accordingly, the peptide molecules of the array include pseudopeptides and peptidomimetics. The peptides can be (a) naturally occurring, (b) produced by chemical synthesis, (c) produced by recombinant DNA technology, (d) produced by biochemical or enzymatic fragmentation of larger molecules, (e) produced by methods resulting from a combination of methods (a) through (d) listed above, or (f) produced by any other means for producing peptides.

A peptide can for example comprise up to 1, 2 3, 4, or up to 5 conservative changes for every 15 amino acid sequence. For example, each peptide can comprise up to 70%, 75%, 80%, 85%, 90%, 95% sequence identity with a peptide selected from Table 1.

The term “sample” as used herein means any biological fluid or tissue sample from a subject, or fraction thereof which can be assayed for kinase activity, including for example, a lysate of a part of an organism or cell population wherein the cell population is obtained from a subject. The sample can, for example comprise a head, thorax or a whole organism (e.g. whole bee). The sample can be an experimental sample treated with a stressor (e.g. infected) or a control that is optionally untreated or treated with a control treatment (e.g. vehicle only). It is disclosed herein that the choice of control can be important in identifying differentially phosphorylated peptides. Depending on the stressor, an appropriate control treatment can be a vehicle only treatment (e.g. stressor dissolution agent) or a control treatment that is similar in composition to the stressor treatment but lacking the specificity of the stressor. For example a control treatment for a macromolecule, such as a peptide or RNA that induces a sequence specific cell response, can comprise a scrambled macromolecule, e.g. sequence scrambled peptide or RNA molecule. Similarly an isotype control antibody can be used as a control treatment wherein the stressor is an antibody. Any population of cells can be treated. For example, the cell or population of cells can comprise subject cells from multiple subjects, each sample optionally corresponding to a different subject, wherein one or more subsets of cells from each subject are treated with a stressor, optionally in vivo (e.g. an animal challenge) or in vitro (e.g. ex vivo treated primary cells). The cells are optionally clonal cells (e.g. cell culture experiment) and comprise propagated cells under defined conditions. Wherein multiple stressors are being compared or when using cells from one or more subjects, a biological control dataset for the same subject and/or sample treatment is optionally obtained and optionally subtracted from an experimental dataset (e.g. a control dataset comprising phosphorylation signal intensities corresponding to an unstimulated level of kinase activity is subtracted from each treatment dataset).

The term “subject” as used herein means any living organism, such as an insect such as silkworm, lac insect and bee, including for example a honey bee and/or related species such as wasp. —The subject can also be for example a eukaryote including any animal or plant, including any crop plant, or a prokaryote.

The term “bee” as used herein means any bee including Apis melifera commonly known as honey bees and closely related species, such as for example A. koschevnikovi, A. cerana, A. nigrocincta, A. nuluensis and A. indica.

In an embodiment, the array comprises one or more assay controls for example one or more negative controls and/or one or more positive controls. In an embodiment, the negative control or negative reference peptide or peptides does not contain any Ser, Thr or Tyr residues. Positive control peptides could include for example peptides comprising phosphorylation sites of histones 1 through 4, bovine myelin basic protein (MBP), and/or α/β casein.

The array can be used to measure protein kinase activity in a bee sample. The array enables for example investigation of phosphorylation-mediated signal transduction activity in bees and can be used to identify biomarkers for marker assisted selection and/or to understand some of the biology associated with particular phenotypes. For example, as demonstrated below, different bee phenotypes, such as susceptible and tolerant to varroa infection, exhibit differences in cellular signalling pathways discernable using an array comprising bee specific peptides comprising known or putative phosphorylation sites. The profiles obtained for a specific phenotype are reproducible and specific profiles can be obtained for use in identifying bees of unknown or otherwise unconfirmed characteristics. The variable, phenotype related, presence of protein kinases and their ability to phosphorylate specific peptides enables the analysis of bee samples and identification of specific characteristics. Furthermore, the peptide arrays described herein can be used to identify honey bee phenotypes quickly.

The term “phenotype” as used herein means a physical, behavioural, developmental, physiological, or biochemical characteristic of an organism, determined by genetic makeup and/or environmental influences.

For example the technology can be applied to honey bee breeding programs and used to identify phenotypes of interest for example susceptibility/resistance to pathogenic organisms and/or cellular responses to infection in honey bees and other organisms.

Accordingly in another aspect, the disclosure includes a method for measuring protein kinase activities in a sample from a subject, said method comprising the steps of: a) incubating a sample obtained from said subject with ATP and/or other suitable ATP source and a plurality of peptides, for example, wherein each of the plurality comprises a sequence of about 5 to about 100 amino acids, for example about 5 to about 50 amino acids or about 5 to about 30 amino acids, wherein the sequence comprises a contiguous sequence present in a peptide sequence selected from Table 1, wherein said contiguous sequence comprises a bee phosphorylation site sequence; and, b) determining a detectable phosphorylation profile, said phosphorylation profile resulting from the interaction of the sample with the plurality of peptides, wherein said phosphorylation profile provides a measure of one or more kinase activities in the sample.

In an embodiment, the method further comprises obtaining a sample from the subject.

The plurality of peptides can comprise for example peptide sequences of a selected group of molecules, for example proteins involved in immune responses, specific signaling cascades or can be related molecules, e.g. sharing a particular sequence identity.

The term “sequence identity” as used herein refers to the percentage of sequence identity between two polypeptide sequences or two nucleic acid sequences. To determine the percent identity of two amino acid sequences or of two nucleic acid sequences, the sequences are aligned for optimal comparison purposes (e.g., gaps can be introduced in the sequence of a first amino acid or nucleic acid sequence for optimal alignment with a second amino acid or nucleic acid sequence). The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position. The percent identity between the two sequences is a function of the number of identical positions shared by the sequences (i.e., % identity=number of identical overlapping positions/total number of positions times 100%). In one embodiment, the two sequences are the same length. The determination of percent identity between two sequences can also be accomplished using a mathematical algorithm. A preferred, non-limiting example of a mathematical algorithm utilized for the comparison of two sequences is the algorithm of Karlin and Altschul, 1990, Proc. Natl. Acad. Sci. U.S.A. 87:2264-2268, modified as in Karlin and Altschul, 1993, Proc. Natl. Acad. Sci. U.S.A. 90:5873-5877. Such an algorithm is incorporated into the blastn and blastp programs of Altschul et al., 1990, J. Mol. Biol. 215:403. BLAST nucleotide searches can be performed with the blastn nucleotide program parameters set, to default parameters or e.g., wordlength=28. BLAST protein searches can be performed with the blastp program parameters set to default parameters, or e.g., wordlength=3 to obtain amino acid sequences homologous to a polypeptide molecule of the present disclosure. To obtain gapped alignments for comparison purposes, Gapped BLAST can be utilized as described in Altschul et al., 1997, Nucleic Acids Res. 25:3389-3402. Alternatively, PSI-BLAST can be used to perform an iterated search which detects distant relationships between molecules (Id.). When utilizing BLAST, Gapped BLAST, and PSI-Blast programs, the default parameters of the respective programs (e.g., of blastp and blastn) can be used (see, e.g., the NCBI website). The percent identity between two sequences can be determined using techniques similar to those described above, with or without allowing gaps. In calculating percent identity, typically only exact matches are counted.

In an embodiment, the plurality of peptides are comprised in an array, for example an array described herein.

In another embodiment, the plurality of peptides is comprised in a composition that is contacted with ATP and/or other suitable ATP source and the level of phosphorylation is detected by a method known in the art. For example, the composition can be separated electrophoretically and probed with a phosphospecific antibody, or visualized using labeled ATP of a phosphor specific stain. Slot blots, immunohistochemical and the like can also be used. This method can be used for example with a subset of peptides and/or corresponding proteins are being assessed for example about 2, 3, 4, 5, 6 to 10, 11-15 or more peptides or corresponding proteins.

A further aspect includes a composition comprising one or more peptides listed in Table 1 and a diluent. The peptide can for example be attached to a bead or spotted on a slide and can for example be used in methods described herein. For example, Table 3 and 4 identify peptides that are differentially phosphorylated in varroa sensitive and tolerant bees. One or more of these peptides could be used as a biomarker for varroa tolerance. In an embodiment, the composition comprises 1 to 288 peptides listed in Table 1, or any number of peptides between 1 and 288. In an embodiment, the one or peptides is selected from Table 2. In another embodiment, the one or more peptides is selected from Table 3. In yet another embodiment, the one or more peptides is selected from Table 4.

Each of the plurality of peptides, whether isolated, in a composition or in an array, can comprise about 5 to about 100 amino acids, for example about 5 to about 50 amino acids or about 5 to about 30 amino acids, wherein the sequence comprises a contiguous sequence present in a peptide sequence selected from the group of SEQ ID NOs: 1 to 288 (e.g. Table 1), wherein the contiguous sequence comprises a bee phosphorylation site sequence.

Developing productive, gentle, honey bee colonies with tolerance to mites and brood diseases is an objective of honey bee breeders and as described herein, the arrays can be used to identify bees with desirable phenotypes. It is demonstrated for example that a phosphorylation profile or signature is associated with varroa sensitive and resistant bee lines and further that infection produces differential responses in these groups.

Accordingly another aspect includes use of a plurality of peptides described herein for example including peptides listed in Tables 1, 2, 3 and/or 4, in a composition or on an array, for example for comparing high and low honey producers, varroa sensitive and tolerant lines and viral sensitive and resistant (immune) lines (e.g. using infection models), or any other phenotype of interest, for differences in phosphorylation of signal transducing molecules (kinome arrays).

It is demonstrated herein, it is believed for the first time, that kinotyping can be used for identifying organism level phenotypes. Organisms such as bees are made up of diverse cell types. It is demonstrated herein that whole organisms and/or parts thereof can be used to identify organism phenotypes by kinome analysis.

Accordingly an aspect of the disclosure includes a method for classifying a subject for example as having or not having a phenotype, the method comprising a) determining a detectable phosphorylation profile of a sample obtained from the subject, said phosphorylation profile resulting from the interaction of the sample with a plurality of peptides described herein; b) comparing said phosphorylation profile to a reference phosphorylation profile of a known phenotype (e.g. a phenotype reference phosphorylation profile); wherein a difference or a similarity in the phosphorylation profile of the plurality of peptides between the sample and the control is used to classify the subject for example as having or not having the phenotype.

In an embodiment, the method comprises: a) determining a detectable phosphorylation profile of a sample obtained from the subject, said phosphorylation profile resulting from the interaction of said sample with a plurality of peptides described herein; b) comparing said phosphorylation profile to one or more reference phosphorylation profiles, each reference phosphorylation profile corresponding to a known phenotype (e.g. a phenotype reference phosphorylation profile); and c) classifying the subject according to the probability of said phosphorylation profile falling within a class defined by said reference phosphorylation profile.

The subject can be classified for example as having or not having a phenotype or classified as having a first or second phenotype.

In an embodiment, the method for classifying a subject for example as having or not having a phenotype, comprises a) obtaining a sample of the subject; b) incubating said sample with ATP and/or other suitable ATP source and a plurality of peptides, for example comprising sequences or parts thereof selected from Table 1 and/or other peptides, each peptide comprising a phosphorylation site sequence; and c) determining a detectable phosphorylation profile, said phosphorylation profile resulting from the interaction of the sample with the plurality of peptides; d) comparing said phosphorylation profile to one or more reference phosphorylation profiles of a known phenotype (e.g. one or more phenotype reference phosphorylation profiles); wherein a difference or a similarity in the phosphorylation profile of the plurality of peptides between the sample and said one or more reference phosphorylation profiles is used to classify the subject for example as having or not having the phenotype.

For example, a subject is identified as having the phenotype associated with a reference phosphorylation profile if the subject phosphorylation profile is similar to said reference phosphorylation profile.

Accordingly, in an embodiment, the method further comprises: identifying the subject as having the phenotype of a phenotype reference phosphorylation profile if said phosphorylation profile is similar to said phenotype reference phosphorylation profile or identifying the subject as not having the phenotype of the phenotype reference phosphorylation profile if the said phosphorylation profile is not similar to said phenotype reference phosphorylation profile: or identifying the subject as having the phenotype corresponding to a first phenotype reference phosphorylation profile if said phosphorylation profile is similar to said first phenotype reference phosphorylation profile or identifying the subject as having the phenotype corresponding to a second phenotype reference phosphorylation profile if said phosphorylation profile is similar to said second phenotype reference phosphorylation profile.

In an embodiment, the similarity is assessed by calculating a measure of similarity.

The subject is identified as having or likely having the phenoytype of the phenotype reference phosphorylation profile most similar to said subject phosphorylation profile. For example, if a subject has a higher similarity to a first phenotype reference phosphorylation profile, the subject is identified as having said first phenotype; if a subject has a higher similarity to a second phenotype reference phosphorylation profile, the subject is identified as having said second phenotype. If determining for example whether the subject The phosphorylation levels can also be used to determine a threshold, wherein if a subject is above or below a threshold, the subject is identified as having the phenotype corresponding to above or below the threshold.

In an embodiment, the disclosure includes a method of classifying a subject as having or not having a phenotype, the method comprising (i) calculating a first measure of similarity between a first phosphorylation profile, said first phosphorylation profile comprising the phosphorylation levels of a plurality of peptides described herein, in a cell sample taken from said subject and a first phenotype reference phosphorylation profile, said first phenotype reference phosphorylation profile comprising phosphorylation levels of said plurality of peptides that are for example, average levels of said respective peptides in cells of a plurality of subjects having said first phenotype; and (ii) classifying said subject as having the first phenotype if said first phosphorylation profile has a similarity to said first phenotype reference phosphorylation profile that is above a predetermined threshold, classifying said subject as not having said first phenotype if said first phosphorylation profile has a similarity to said first phenotype reference phosphorylation profile that is below a predetermined threshold,

In an embodiment, step (i) further comprises: calculating a second measure of similarity between said first phosphorylation profile and a second phenotype reference phosphorylation profile, said second phenotype reference phosphorylation profile comprising phosphorylation levels of said plurality of peptides that are average phosphorylation levels of the respective peptides in cells of a plurality of subjects having said second phenotype; and classifying said subject as having said second phenotype if said first phosphorylation profile has a similarity to said first phenotype reference phosphorylation profile that is below a predetermined threshold and said first phosphorylation profile has a similarity to said second phenotype reference phosphorylation profile that is above a predetermined threshold.

The phenotype to be assessed can be the presence of a desired trait such as varroa or other pathogen tolerance, increased honey production and/or increased winterability.

In an embodiment, said first phenotype is varroa sensitivity (or pathogen sensitivity) and said second phenotype is varroa tolerance (or pathogen tolerance). In another embodiment, said first phenotype is high honey producer and said second phenotype is low honey producer.

In a further embodiment, the method includes displaying; or outputting to a user interface device, a computer-readable storage medium, or a local or remote computer system, the classification produced by said classifying step.

A further aspect comprises a method of selecting bees with a desired phenotype, the method comprising classifying a subject or subjects from a group of bees (e.g. from a bee colony) as having or not having a phenotype or having a first or second phenotype and selecting members of said group of bees (e.g the same bee colony) with the desired phenotype. The bees can be selected for example for breeding.

It is demonstrated, for example that an array comprising peptides listed in Table 1, was able to distinguish varroa sensitive and varroa tolerant bee lines both in infected and uninfected samples. The peptides listed in Table 2A showed increased phosphorylation when contacted with a sample from varroa sensitive bees compared to when contacted with a sample from tolerant bees and Table 2B showed decreased phosphorylation (e.g. tolerant bees showed increased phosphorylation of Table 2B peptides and decreased phosphorylation of Table 2A peptides compared to sensitive bees). This increased phosphorylation was detectable in both infected bees and in uninfected bees. Table 3A lists peptides whose phosphorylation was increased by contact with infected G4 sensitive bee samples compared to infected tolerant S88 bee samples while Table 3B lists peptides with decreased phosphorylation in sensitive bees compared to tolerant bees (e.g. tolerant bees showed increased phosphorylation of Table 3B peptides and decreased phosphorylation of Table 3A peptides compared to sensitive bees). Table 4A lists peptides that were increased in uninfected G4 sensitive bees compared to uninfected tolerant S88 bees while Table 4B lists peptides with decreased phosphorylation in uninfected sensitive bees compared to uninfected tolerant bees (e.g. tolerant bees showed increased phosphorylation of Table 4B peptides and decreased phosphorylation of Table 4A peptides compared to sensitive bees). Accordingly, a phosphorylation profile most similar to a reference phosphorylation profile associated with tolerance for example a phosphorylation profile for a plurality of peptides with similar direction and/or magnitude of increases or decreases as shown in Tables 3 or 4 for varroa tolerant bees, is indicative that the bee line tested will exhibit varroa tolerance and detecting a phosphorylation profile most similar to a profile associated with varroa sensitivity, for example a phosphorylation profile for a plurality of peptides with similar direction and/or magnitude of increases or decreases as shown in Tables 3 or 4 for varroa sensitive bees, is indicative that the bee is likely varroa sensitive.

Accordingly, in another aspect the disclosure includes a method for identifying a biomarker in a subject associated with a desirable phenotype, the method comprising:
a) obtaining a sample from the subject;
b) contacting the sample with ATP and/or other suitable ATP source and a plurality of peptides comprising peptides (or parts thereof comprising phosphorylation site sequences) selected from Table 1;
c) determining a phosphorylation profile of the plurality of peptides;
d) comparing the phosphorylation profile of the plurality of peptides with a control; wherein a difference or a similarity in the phosphorylation profile of the plurality of peptides between the sample and the control is used to identify a biomarker and/or set of biomarkers associated with a desirable phenotype.

For example, a highly phosphorylated peptide and/or set of peptides (e.g. phosphorylation profile) can identify a signaling molecule or signaling pathway that is associated with the desirable phenotype.

The arrays and methods can for example identify biomarkers and/or profiles associated with high honey producers and/or mite and virus resistant lines.

Accordingly, in an embodiment, the desirable property is pathogen resistance, increased honey production and/or increased winterability.

In an embodiment, the method can involve a treatment such as a pathogen challenge. For example, in an embodiment the method comprises:
a) obtaining a sample from a subject treated with a stressor;
b) contacting the sample with ATP and/or suitable ATP source and a plurality of peptides comprising peptides selected from Table 1;
c) determining a phosphorylation profile of the plurality of peptides;
d) comparing the phosphorylation profile of the plurality of peptides with a control;
   wherein a difference or a similarity in the phosphorylation profile of the plurality of peptides between the sample and the control is used to identify a biomarker and/or set of biomarkers associated with the desirable phenotype.

A compound that functions as ATP can also be used instead of ATP in the methods described. For example, other suitable ATP sources such ATP analogs can be used. GTP can also be used in place of ATP or ATP source.

Detecting the phosphorylated biomarker is indicative that a subject has an increased likelihood of having the phenotype associated with the biomarker (e.g. increased or decreased phosphorylation compared to a control not having the desired phenotype).

The sample from the subject can alternatively be a cell sample from a cell line, for example treated with a stressor.

In an embodiment, the pathogen resistance is viral resistance such as Cripaviridae, Dicistroviridae, Iflaviridae and Irroviridae resistance; parasite resistance such as varroa mite resistance, microspordia resistance (e.g. Nosema tolerance), tracheal mite resistance, hive beetle resistance, and wax moth resistance; bacterial resistance, such as resistance to foulbrood causing bacteria; and fungal resistance, such as resistance to chalkbrood and stone brood causing fungi.

In another embodiment, the method further comprises selecting the subject with the desirable property for example for breeding.

The arrays can for example be used in monitoring the innate immune response to microbial infections in the honey bee and differentiating between pathogen susceptible and resistant lines.

In an embodiment, the disclosure includes a method of screening for subject susceptibility and/or resistance to a pathogen, the method comprising:
a) obtaining a sample from a subject;
b) contacting the sample with ATP and/or other suitable ATP source and a plurality of peptides comprising peptide sequences selected from Table 1;
c) determining a phosphorylation profile of the plurality of peptides;
d) comparing the phosphorylation profile of the plurality of peptides with one or more reference phosphorylation profiles;
   wherein a difference or a similarity in the phosphorylation profile of the plurality of peptides between the sample and the reference phosphorylation profiles identifies the subject as susceptible or resistant to pathogen.

In another aspect, the disclosure includes a method of aiding selection of a subject with a desirable phenotype comprising:
a) determining a subject phosphorylation profile from a test sample of the subject;
b) providing one or more reference phosphorylation profiles associated with a known phenotype, wherein the subject phosphorylation profile and the reference phosphorylation profile(s) have one or a plurality of values, each value representing a phosphorylation level of a peptide selected from the peptides in Table 1; and
c) identifying the reference phosphorylation profile most similar to the subject phosphorylation profile,
   wherein the subject is predicted to have the phenotype of the reference phosphorylation profile most similar to the subject phosphorylation profile.

For example, each value representing a phosphorylation level of a peptide selected from Table 1 can include phosphorylation data obtained using the peptide and/or obtained in the context of the corresponding protein comprising the corresponding phosphorylation site.

For example, the level of phosphorylation of the peptide is used as a surrogate marker of the level of phosphorylation of the corresponding protein.

In an embodiment, the subject is a bee, such as a honey bee.

In an embodiment, the method comprises screening for bee susceptibility and/or resistance to varroa infection.

In an embodiment, wherein the subject is infected with varroa, decreased phosphorylation of 2 or more peptides in Table 2A and/or 3A associated with varroa resistance and/or increased phosphorylation of 2 or more peptides in Table 2B and/or 3B is indicative that the subject is varroa resistant.

If the subject is uninfected, differential phosphorylation of 2 or more peptides in Table 2A and/or 4A associated with varroa resistance and/or 2 or more peptides in Table 2B and/or 46 associated with varroa resistance is indicative the subject is varroa resistant. For example decreased phosphorylation of 2 or more peptides in Table 2A and/or 4A associated with varroa resistance and/or increased phosphorylation of 2 or more peptides in Table 2B and/or 4B associated with varroa resistance is indicative the subject is varroa resistant.

In an embodiment, the method is used to determine a phosphorylation profile associated with Nosema apis infections, which is a microsporidium parasite that affects honey bees.

In an embodiment, bees identified as pathogen resistant such as varroa resistant are selected for breeding. In an embodiment, the methods and/or arrays described herein are used to assess miticide effectiveness. In another embodiment, varroa resistant infected bees that respond to miticide are treated with mitocide, for example to manage varroa population growth. For example, honey bees show varying degrees of tolerance to varroa. Phenotypes showing more tolerance typically respond better to mitocide treatment.

The term “control” as used herein refers to a sample or samples of subjects e.g. whole bees, with a known phenotype, or a fraction of such a sample thereof such as but not limited to, head protein extract and/or thorax extract, and/or a reference phosphorylation profile comprising numerical value and/or ranges (e.g. control range) corresponding to the phosphorylation level of a plurality of peptides in such a sample or samples (e.g. average, median, cut-off value etc.). The control can for example be a set of numerical values corresponding to and/or derived from the phosphorylation levels of a plurality of peptides of a known phenotype and/or treatment response that is predetermined. Comparison to a phenotype reference phosphorylation profile can comprise obtaining the phenotype reference phosphorylation profile, for example obtaining one or more controls with known phenotype, and determining a phosphorylation profile that comprises members with the known members, for example with a selected a p-value or within 1 or 2 standards of deviation.

For example, the control (or phenotype reference phosphorylation profile associated therewith) can be a selected cut-off or threshold level, or control score comprising for example a desired specificity above which a subject bee line is identified as having the phenotype being assessed, e.g. corresponding to a median level in a population. For example, a test subject that has an increased level of phosphorylation for a plurality of peptides above a cut-off, threshold level or control score is indicated to have or is more likely to have the known phenotype e.g. varroa resistance.

The cut-off, threshold or control score can for example be a median level or value, or composite score comprising the median phosphorylation level or levels of a plurality of peptides. The threshold can be selected to optimize the trade-off between false negative and false positive discoveries. It may also be desirable to define multiple thresholds, corresponding to for example the penetrance of the phenotype (e.g. strongly varroa resistant, intermediate varroa resistance).

The term “control level” refers to a peptide phosphorylation signal intensity in a control sample or a numerical value corresponding to such a sample (e.g. in a reference phosphorylation profile). Control level can also refer to for example a threshold, cut-off or baseline level of a peptide phosphorylation associated with a particular phenotype.

The term “determining a phosphorylation level” or “determining a phosphorylation profile” as used herein means the application of a reagent such as a peptide, or a plurality of peptides, to a sample, for example a sample of the subject bee line and/or a control sample, for ascertaining or measuring quantitatively, semi-quantitatively or qualitatively the amount of peptide phosphorylation signal intensity. For example, the plurality of peptides can be comprised in an array (e.g. on a slide or beads) as described herein and phosphorylation specific stains such as fluorescent ProQ Diamond Phosphoprotein Stain (Invitrogen) and Stains-All” (1-ethyl-2-[3-(3-ethylnaphtho[1,2]thiazolin-2 ylidene)-2-methylpropenyl]-naphtha[1,2]thiazolium bromide) and/or labeled ATP such as radiolabelled ATP can be used to detect phosphorylation. The phosphorylation signal can be detected by a number of methods known in the art such as using phosphospecific antibodies directly or indirectly labeled and/or using a method disclosed herein.

For example a phosphospecific detection agent such as an antibody, for example a labeled antibody, which specifically binds the phosphorylated forms of peptides, can be used for example to detect relative or absolute amounts of peptide phosphorylation.

The term “difference in the level” as used herein in comparison to a control (e.g. or to a phenotype reference phosphorylation profile) refers to a measurable difference in the level or quantity of peptide phosphorylation in a test sample, compared to the control that is of sufficient magnitude to allow assessment, for example of a statistically significant difference. The magnitude of the difference is sufficient for example to determine that the subject falls within a class of subjects likely to have the phenotype of the control population being tested e.g. fall within the class defined by the phenotype phosphorylation profile. For example, a difference in a level of peptide phosphorylation is detected if a ratio of the level in a test sample as compared with a control is greater than 1.2. For example, a ratio of greater than 1.3, 1.4, 1.5, 1.6, 1.7, 2, 2.5 or 3 or more and/or has a p-value of less than 0.1, 0.05 or 0.01.

The term “phosphorylation level” as used herein in reference to a peptide phosphorylation refers to a phosphorylation signal intensity that is detectable or measurable in a sample and/or control.

The term “phosphorylation profile” or “subject phosphorylation profile” as used herein refers to, for a plurality (e.g. at least 2, for example 5) of peptides and/or their corresponding proteins, phosphorylation signal intensities detectable after contacting a sample from a subject with the plurality of peptides under conditions that permit peptide phosphorylation as would be known to a person skilled in the art (e.g. temperature, buffer constituents, presence of ATP and/or other suitable ATP source etc.). The plurality of peptides optionally comprises at least 2, at least 3, at least 4, at least 5, or more of the peptides listed in Table 1, including for example any number of peptides between 2 and 288.

For example, the assessment of similarity can comprise identifying peptides (or profiles) with phosphorylation levels which meet a specific threshold such as have a minimum p-value and/or fold change. For example, for varroa resistance, the subset can comprise peptides listed in Tables 3 and 4 that have a greater fold increase than a selected threshold, for example, greater than 1.5 fold change, or greater than a 2 fold change or a p-value below a selected value such as 0.1, 0.5 and/or 0.01. In an embodiment, the plurality of peptides assessed comprises the 2, 3, 5, 10, 15, or 20 peptides (or any number of peptides between 2 and 288) with the greatest fold increase or smallest p-value listed in Tables 3 and 4.

The term “measuring” or “measurement” as used herein refers to the application of an assay to assess the presence, absence, quantity or amount (which can be an relative or absolute amount) of either a given substance within a subject-derived sample, including the derivation of qualitative or quantitative concentration levels of such substances.

The term “reference phosphorylation profile” or “phenotype reference phosphorylation profile” as used herein refers to a suitable comparison profile, for example which comprises the phosphorylation characteristics of a plurality of peptides, for example selected from the peptides listed in Tables 1, 2, 3 and/or 4, associated with a particular phenotype. For example, Tables 2, 3 and 4 list peptides whose phosphorylation is significantly different in varroa sensitive versus tolerant bees (e.g. Table 2), infected varroa sensitive versus infected tolerant bees (Table 3) and uninfected varroa sensitive versus uninfected tolerant bees (Table 4). Accordingly, the table provides profiles for varroa sensitive and tolerant bee lines. The reference phosphorylation profiles are compared to subject phosphorylation profiles for a plurality of peptides). A subject can be classified by comparing to a phenotype reference phosphorylation profile, where the phenotype reference phosphorylation profile most similar to the subject profile is indicative that the subject is likely to express the phenotype associated with the phenotype reference phosphorylation profile. The phenotype reference phosphorylation profile can be derived for example from the same sample type as the subject sample (e.g. whole organism, or part such as head or thorax).

The term “similar” in the context of a phosphorylation level as used herein refers to a subject phosphorylation level for a peptide that falls within the range of levels associated with a particular class for example associated with varroa tolerance (e.g. and not varroa sensitivity). Accordingly, “detecting a similarity” refers to detecting a phosphorylation level (or levels) that falls within the range of levels associated with a particular class. In the context of a reference phosphorylation profile, a subject profile is “similar” to a reference phosphorylation profile associated with a phenotype such as varroa tolerance if the subject profile shows a number of identities and/or degree of changes (e.g. in terms of direction of phosphorylation (increased or decreased) and/or magnitude) with the reference phosphorylation profile.

The term “most similar” in the context of a reference phosphorylation profile refers to a reference phosphorylation profile that shows the greatest number of identities and/or degree of changes with the subject phosphorylation profile.

Similarity can be determined for example using clustering analysis.

Similarity can also be determined by calculating a similarity score or threshold.

A further aspect includes a kit comprising a plurality of peptides described herein comprising sequences present in a peptide selected from Table 1, an array comprising a support and the plurality of peptides, and/or a kit control.

In an embodiment, the kit further comprises instructions for use.

In an embodiment, the kit comprises about 5, 10, 15, 20, 25, 30, 35, 40, 50, 60, 70, 80, 90, 100, 125, 150, 175, 200, 225, 250, 275, 300 or more peptides.

The term “kit control” as used herein means a suitable assay standard or reference reagent useful when determining a phosphorylation level of a peptide, for example a peptide that known to be phosphorylated or not phosphorylated under the conditions of the assay or for example a peptide corresponding to a substrate of a kinase with constitutive activity.

Another aspect includes a phosphorylation profile comprising for each of a plurality of peptides, one or more phosphorylation characteristics, for example signal intensities, fold change, and/or phosphorylation status, associated with a phenotype and/or treatment.

In an embodiment, the phosphorylation profile comprises for a plurality of peptides, one or more of phosphorylation status, fold change, and/or p-value associated with a fold change listed in Table 2 and/or 3. The phosphorylation profile can for example serve as a reference phosphorylation profile for comparing subject profiles when assessing, as in the present Tables, varroa resistance or lack thereof.

The plurality of peptides and/or an array comprising the plurality of peptides can be analysed to obtain a phosphorylation profile using a number of methods. For example, the signal intensities measuring specific phosphorylation events of the peptides on a kinome array are subjected to variance stabilization transformation to bring all the data onto the same scale while alleviating variance-mean-dependence. Spot-spot and subject-subject variability are examined using χ² and F-tests to identify and eliminate inconsistently regulated peptides due to technical and biological factors of the experiments, respectively. One-sided paired t-test is used to identify differentially phosphorylated peptides relative to the control from the preprocessed kinome data. The information from the differential peptides can be used to probe gene ontology (GO) annotations and known signaling transduction pathways from online database to discover treatment-specific cellular events from various biological aspects. For comparative visualization of the global kinome profiles induced by selected stimuli, hierarchical clustering and principal component analysis are applied to the data after averaging the replicate intensities. The results from the differential analyses and clustering are compared to draw further insights from the data and/or to classify subjects. The results can be presented for example in pseudo-images generated based on the p-values from the one-sided t-tests for phosphorylation or dephosphorylation of each peptide. Each peptide is represented for example by one small colored circle. The depths of the coloration in the colors, for example red and green, are inversely related to the corresponding p-values.

In an embodiment, the phosphorylation profile is determined by analyzing the phosphorylation data of a plurality of peptides, the method comprising:
a. obtaining one or more datasets, each dataset comprising a phosphorylation signal intensity for each peptide of the plurality of peptides;
b. transforming the phosphorylation signal intensity of each peptide using a variance stabilizing transformation to provide a variance stabilized signal intensity for each peptide of the plurality of peptides;
c. identifying one or more peptides of the plurality of peptides that are consistently phosphorylated or consistently unphosphorylated,
   thereby providing a phosphorylation profile.

In an embodiment, the phosphorylation data is bee kinome data.

The term “signal intensity” as used herein refers to a value such as a numerical value corresponding to the strength of a specific signal being measured. For example, “phosphorylation signal intensity”, refers to a value corresponding to the strength of the phosphorylation signal being measured. When referring to a phosphorylation signal intensity of a peptide on an array, the signal intensity is a value corresponding, for example, to the signal intensity of the “spot” where the peptide is spotted on the array.

Each peptide in the dataset can be represented by one or more replicates. In an embodiment, each peptide of the plurality is present in at least 1 replicate, at least 2 replicates, at least 3 replicates, at least 4 replicates, at least 5 replicates, at least 6 replicates, at least 7 replicates, at least 8 replicates, at least 9 replicates, at least 10 replicates, at least 12 replicates, or at least 15 replicates.

In an embodiment, the step of identifying the one or more peptides comprises calculating a phosphorylation consistency value for each peptide of the plurality of peptides.

In an embodiment, the phosphorylation consistency value is calculated using the variance stabilized signal intensity.

In another embodiment, the phosphorylation profile is determined by analyzing phosphorylation data of a plurality of peptides, each peptide of the plurality present in at least two replicates, the method comprising:
a. obtaining one or more datasets, each dataset comprising a phosphorylation signal intensity for each replicate of the plurality of peptides;
b. transforming the phosphorylation signal intensity of each replicate using a variance stabilizing transformation to provide a variance stabilized signal intensity for each replicate of the plurality of peptides;
c. identifying one or more peptides of the plurality of peptides that are consistently phosphorylated or consistently unphosphorylated by calculating a phosphorylation consistency value for each peptide of the plurality of peptides, the phosphorylation consistency value optionally comprising calculating a replicate variability for each peptide using the variance stabilized signal intensity of each replicate of the at least two replicates for each peptide.

In an embodiment, the phosphorylation consistency value is calculated using a chi-square (χ²) statistic. In another embodiment, the method further comprises determining a phosphorylation characteristic of at least one of the one or more peptides that are consistently phosphorylated or consistently unphosphorylated.

A peptide is identified as consistently phosphorylated or consistently unphosphorylated according to the phosphorylation consistency value. Under the same treatment conditions, peptides with a phosphorylation consistency value such as a p-value which is for example, less than a threshold, are identified as inconsistently phosphorylated and peptides with a phosphorylation consistency value which is greater than a threshold are identified as consistently phosphorylated or consistently unphosphorylated. A person skilled in the art would recognize depending on the phosphorylation consistency value calculated, in some instances the opposite applies—peptides with a phosphorylation consistency value greater than a threshold are identified as inconsistently phosphorylated and peptides with a phosphorylation consistency value which is less than a threshold are identified as consistently phosphorylated or consistently unphosphorylated.

A phosphorylation characteristic is determined for at least one of the one or more peptides consistently phosphorylated or consistently unphosphorylated.

As used herein the term “phosphorylation characteristic” means a value, feature or quality that is distinctive of a peptide that relates to its phosphorylation. For example, the phosphorylation characteristic can include but is not limited to the phosphorylation status of the peptide, the phosphorylation consistency value, the location of the peptide on the peptide array, the sequence of the peptide, the phosphorylation signal intensity or the variance stabilized signal intensity or any other property of the consistently phosphorylated or consistently unphosphorylated peptide related to phosphorylation of the peptide. Depending on the desired phosphorylation characteristic, the characteristic can be determined by identifying for example, the sequence, or calculating the variance stabilized signal intensity.

In an embodiment, the method further comprises outputting the phosphorylation characteristic of one or more of the plurality of peptides, optionally a phosphorylation status and/or the phosphorylation consistency value. In an embodiment, the method comprises outputting a phosphorylation characteristic of one of the one or more peptides that is/are consistently phosphorylated or consistently unphosphorylated.

The dataset is generated in an embodiment, using at least one peptide array probed with a sample, wherein each peptide of the plurality of peptides is present on each peptide array in at least one, at least 2 replicates (e.g. each peptide is spotted at least twice) or at least 3 replicates (e.g. each peptide is spotted thrice). The peptide can be spotted 4, 5, 6, 7, 8, 9 or more times. Multiple arrays can also be utilized.

The term “a replicate” with respect to a peptide as used herein refers to a peptide that has the same sequence and length as another peptide (e.g. two peptides having the same sequence and length are replicates of each other) treated under the same conditions (e.g. contacted with the same sample). The replicates can for example, be spotted on a same peptide array, or spotted on separate arrays wherein each array is contacted with the same sample (e.g. an aliquot of the same sample, e.g. same treatment same subject).

As used herein “replicate variability” also referred to as “spot-spot variability” refers to variability among replicates (e.g. spots on a peptide array) corresponding to the same treatment (e.g. stressor or control treatment).

In an embodiment, each dataset corresponds to a sample (e.g. a treatment and/or subject). In an embodiment, the sample is an experimental sample treated with a stressor or a control sample. In an embodiment, the method comprises:
a) obtaining one or more datasets, each dataset comprising a phosphorylation signal intensity for each replicate of the plurality of peptides for a sample, wherein the dataset is generated using at least one peptide array probed with the sample, wherein each peptide of the plurality of peptides is present on each peptide array in at least 2 replicates and wherein the sample is optionally an experimental sample treated with a stressor or a control sample;
b) transforming the phosphorylation signal intensity of each replicate of the plurality of peptides using a variance stabilizing transformation to provide a variance stabilized signal intensity for each replicate of the plurality of peptides;
c) identifying one or more peptides of the plurality of peptides that is/are consistently phosphorylated or consistently unphosphorylated by calculating a phosphorylation consistency value for each peptide of the plurality of peptides for each sample, wherein the phosphorylation consistency value is a measure of the phosphorylation status variability among the replicates for each peptide and optionally comprises calculating a replicate variability for each peptide using the variance stabilized signal intensity of each replicate, optionally using a chi-square (χ²) statistic;
d) determining a phosphorylation characteristic of at least one of the one or more peptides that is/are consistently phosphorylated or consistently unphosphorylated; and
e) optionally outputting a phosphorylation characteristic of the one or more of the plurality of peptides, for example a phosphorylation characteristic of one of the one or more peptides that is/are consistently phosphorylated or consistently unphosphorylated.

Phosphorylation data is analysed for example, to determine a phosphorylation characteristic of at least one peptide of the dataset such as the phosphorylation status and/or the phosphorylation consistency value of one or more of the plurality of peptides. In an embodiment, the method comprises determining a phosphorylation status of one or more of the plurality of peptides.

As used herein “phosphorylation status” refers to whether a peptide, polypeptide and/or specific amino acid, such as a peptide on a peptide array, is phosphorylated or unphosphorylated. The phosphorylation status can be determined for example after contact with a sample (e.g. stressor treated or control). The status can for example be an absolute status or a relative status for example relative to a peptide contacted with another sample such as a control or a sample treated with a stressor for a different length of time, e.g. previous time point. When relative to another sample such as a control “unphosphorylated” can include peptides that are “dephosphorylated” (e.g. phosphorylated in a first sample and unphosphorylated in the in the comparator sample). Accordingly, phosphorylation status can further include an indication of whether a peptide is dephosphorylated for example, as a result of a treatment.

The phosphorylation dataset comprises signal intensities (e.g. spot signal intensities) of phosphoimage data measuring specific phosphorylation events for a plurality of peptides, the dataset optionally obtained using a peptide array incubated with a sample using, for example, a microarray scanner and/or a phosphoimager scanner. For example, the peptide array is incubated with a sample such as a treated sample, e.g. treated with a stressor, or a control sample. The peptide array is washed and phosphorylation signal intensity data is captured. The signal intensities are obtained and the captured images processed according to methods known in the art. For example as described in Jalal et al. 2009 (37) sections relating to “using peptide arrays for kinome analysis” incorporated herein by reference, a Typhoon scanner can be set for example at the highest sensitivity setting with a pixel size of 25 microns and used to obtain array images from a phosphoimager screen. The captured image of the phosphoimager screen can be processed using for example ImageQuant TL v2005 software and the images can be cropped to the visible outlines of the peptide arrays in order to obtain individual peptide array images. The coordinates of each spot and the measurements of spacing between spots and blocks, as well as the dimension of spots and blocks can be obtained using, for example Array Vision. The background intensity for each spot can be calculated optionally as the average of pixels from a selected number of regions, such as 4 regions in the immediate vicinity of each spot. The dataset obtained for use in the methods described herein can optionally comprise phosphorylation signal intensity wherein the background intensity has already been subtracted and/or comprise a foreground signal intensity wherein the background intensity is subtracted prior to transformation.

As used herein, “background intensity” with respect to a peptide array signal intensity means the intensity of any non-specific signal that is detectable, for example in regions of the peptide array or array that are adjacent to the spotted peptides.

As used herein, “foreground intensity” with respect to a peptide array signal intensity means a raw signal intensity that is measured for the area which constitutes a spot on the array or array image. A foreground intensity for example can be subtracted for a background intensity (e.g. foreground intensity—background intensity) to provide a phosphorylation signal intensity usable in the methods described herein. For example, the genepix program which can be used to “read” the array image can collect a foreground signal intensity and background level for each individual spot. The raw data file then contains mean intensity of the spot foreground intensity and mean intensity of the background. To obtain a phosphorylation signal intensity, one subtracts the background from the foreground spot signal. In an embodiment, the background is subtracted from the foreground intensity as a first step of the method.

In an embodiment, one or more of the phosphorylation datasets comprises foreground phosphorylation signal intensities and the phosphorylation signal intensity for each replicate is obtained by subtracting a background phosphorylation intensity from each foreground phosphorylation signal intensity to provide the dataset comprising phosphorylation signal intensities for transformation.

The dataset comprises signal intensities measuring specific phosphorylation events of the peptides on the peptide array. Each dataset is subjected to a “preprocessing step” where the signal intensity of each replicate is subjected to a variance stabilizing and normalization (VSN) transformation to bring all the data onto the same scale and to alleviate variance mean dependence. The VSN transformation model can be trained for example using relevant datasets (e.g. similar cell or subject datasets). In an embodiment, R package vsn can be used for the VSN transformation.

The R package or R environment is a software environment for statistical computing and graphics that is publicly available (39).

Following the preprocessing step, the replicate variability such as spot-spot variability is examined, optionally using a chi square test (χ²) to provide a phosphorylation consistency measure for each peptide. Where the number of replicates for a treatment is less than 6, χ² would not be reliable and would be omitted. Other tests for calculating replicate variability include but are not limited to F-test.

The phosphorylation consistency value comprises a measure of the phosphorylation status variability among the replicates for each peptide (e.g. variability in whether the replicates of a peptide are consistently unphosphorylated or phosphorylated) and optionally comprises calculating a replicate variability for each peptide for each sample, wherein the replicate variability is calculated using the variance stabilized signal intensity of each replicate of each peptide, optionally using a chi-square (χ²) statistic. For example, the null hypothesis H₀ claims that there is no difference among intensities from replicate spots, and the alternative hypothesis H_{A} states that there exists significant variation among the replicates. After calculating a phosphorylation consistency value, the consistency of the phosphorylation status among replicates is determined by determining if the phosphorylation consistency value is above a selected threshold. For example, using χ² a p-value is calculated for peptides for the same treatment conditions (e.g. for all replicates of peptides on same or different arrays incubated with a sample treated with the same stressor), and peptides with a p-value less than a selected threshold are considered inconsistently phosphorylated across the spots and are eliminated from any subsequent clustering analysis. Peptides with a p-value above the threshold are considered consistently phosphorylated or consistently unphosphorylated. A desired p-value is selected; for example 0.05, 0.04, 0.03, 0.02 or 0.01 may be selected depending for example on the nature of the experiment. Other optional p-values typically range from 0.05 to 0.01.

The method can be used to analyse and/or compare phosphorylation data of more than one sample. For example, the method can be used to compare an experimental sample to a control sample, and/or multiple experimental samples to each other and/or a control.

Where the samples are from more than one subject of a given species or strain of a species or different individuals, inter-subject variability can confound results. In embodiments where subject variability is a concern, for example in treatments involving outbred animals, the phosphorylation consistency value comprises determining inter-sample or subject variability (such as animal-animal variability), optionally using a F-test statistic. Other tests can also be applied to determine subject variability including but not limited to t-test (i.e. pairwise comparison).

For example, where a dataset for each of three subjects for each of 4 treatments are being compared, the null hypothesis H₀ claims that the mean phosphorylation intensities for the identical peptide from the three animals are the same, and alternative hypothesis H_{A} states that not all three means are equal. The peptides with a p-value greater than a selected consistency threshold are considered consistently phosphorylated or consistently unphosphorylated and peptides with a p-value less than a selected consistency threshold are considered inconsistently phosphorylated and are eliminated from subsequent analysis.

Accordingly in an embodiment, the phosphorylation consistency value is expressed as a p-value. In an embodiment, the selected consistency threshold is a p-value of 0.05, 0.04, 0.03, 0.02 or, 0.01. Other p-values can be chosen depending on the nature the experiment. A typical range of the p-value is from 0.05 to 0.001. The strict confidence level is used so that as much data as possible is retained.

In an embodiment, the phosphorylation consistency value includes calculating the replicate variability and/or the subject variability, using a χ² test to assess the replicate variability and a F-test to assess the subject variability.

In an embodiment, multiple experimental samples are compared. In an embodiment, a biological control signal intensity is subtracted from the experimental signal intensity. In an embodiment, the one or more datasets includes a control dataset and an experimental dataset, a control variance stabilized signal intensity for each replicate of the plurality of peptides is calculated for the control dataset according to a method described herein and subtracted from the variance stabilized signal intensity of each corresponding replicate of the plurality of peptides the experimental dataset prior to determining the subject-subject variability.

In an embodiment, the method comprises identifying peptides that are consistently phosphorylated or consistently unphosphorylated. Accordingly in an embodiment, the method comprises filtering the plurality of peptides according to the phosphorylation status and/or the phosphorylation consistency value and identifying one or more consistently phosphorylated or consistently unphosphorylated peptides. A peptide is identified as consistently phosphorylated or consistently unphosphorylated based on the phosphorylation consistency value, for example, if the phosphorylation consistency value for the peptide is above a selected consistency threshold.

In an embodiment, the disclosure includes a method of identifying one or more peptides of a plurality of peptides that are phosphorylated or unphosphorylated, each peptide of the plurality present in at least two replicates, the method comprising:
a. obtaining one or more datasets, each dataset comprising a phosphorylation signal intensity for each replicate of a plurality of peptides for a sample, the dataset is generated using at least one peptide array probed with the sample;
b. transforming the signal intensity of each replicate of the plurality of peptides using a variance stabilizing transformation to provide a variance stabilized signal intensity for each replicate of the plurality of peptides;
c. determining a phosphorylation consistency value for each peptide of the plurality of peptides wherein the phosphorylation consistency value is a measure of the phosphorylation status variability among replicates and optionally comprises assessing replicate variability of variance stabilized signal intensities using a χ² statistic and/or determining inter-sample variability (such as animal-animal variability for a particular treatment) optionally using an F-test statistic; and
d. identifying one or more peptides identified as consistently phosphorylated or consistently unphosphorylated,
wherein a peptide is identified as consistently phosphorylated or consistently unphosphorylated if the phosphorylation consistency value for the peptide is above a selected consistency threshold.

in an embodiment, the method additionally comprises outputting at least one of the one or more peptides consistently phosphorylated or consistently unphosphorylated. In embodiment, the method comprises outputting a set of peptides consistently phosphorylated or consistently unphosphorylated.

In certain embodiments, the method entails identifying peptides that are differentially phosphorylated or unphosphorylated (e.g. dephosphorylated) compared to another sample (e.g. a control sample). Accordingly another aspect includes a method of identifying one or more peptides differentially phosphorylated in an experimental sample compared to a control sample, the method comprising:
a. for a plurality of peptides, each peptide of the plurality present in at least two replicates,
i. obtaining an experimental dataset, the experimental dataset comprising an experimental phosphorylation signal intensity for each replicate of the plurality of peptides, and
ii. obtaining a control dataset, the control dataset comprising a control phosphorylation signal intensity for each replicate of a plurality of peptides;
b. obtaining a variance stabilized signal intensity for each replicate of one or more peptides of:
i. the experimental dataset identified as consistently phosphorylated or consistently unphosphorylated according to a method described herein, thereby providing a variance stabilized experimental signal intensity for each replicate;
ii. the control dataset identified as consistently phosphorylated or consistently unphosphorylated according to a method described herein, thereby providing a variance stabilized control signal intensity for each replicate;
c. for each peptide that is identified as consistently phosphorylated or consistently unphosphorylated in the experimental dataset and consistently phosphorylated or consistently unphosphorylated in the control dataset, calculating a treatment variability value between the variance stabilized experimental signal intensity and the variance stabilized control signal intensity, optionally using a one-sided t-test; and
d. identifying one or more peptides that is/are differentially phosphorylated in the experimental sample compared to the control sample.

In an embodiment, the experimental dataset is generated using at least one experimental peptide array probed with the experimental sample (e.g. unknown phenotype) and the control phosphorylation signal intensities are generated using at least one control peptide array probed with the control sample (e.g. known phenotype). Alternatively, the control phosphorylation intensities are obtained from a preexisting control phosphorylation profile. In an embodiment, the experimental peptide array and the control peptide array have a common set of peptides. In another embodiment, each peptide of the plurality of peptides is spotted on each peptide array in at least 2 replicates.

In embodiments where the variability value is expressed as a p-value such as when using a one sided t-test, a peptide is differentially phosphorylated, if the peptide has a p-value less than a selected treatment and/or phenotype variability threshold. In an embodiment, the selected treatment variability threshold is 0.2, 0.1, 0.05, or 0.01. Other p-values can be chosen depending on the nature the experiment. A typical range of the p-value is from 0.2 to 0.01.

In an embodiment, the method of identifying one or more peptides that are differentially phosphorylated in an experimental sample treated with a stressor compared to a control sample, comprises:
a. for a plurality of peptides, each peptide of the plurality present in at least two replicates,
i. obtaining an experimental dataset comprising experimental phosphorylation signal intensity for each replicate of a plurality of peptides;
ii. obtaining a control dataset comprising a control phosphorylation signal intensity for each replicate of a plurality of peptides;
b. transforming the signal intensity of each replicate of the plurality of peptides using a variance stabilizing transformation to provide a variance stabilized experimental signal intensity for each replicate of the plurality of peptides of the experimental dataset and a variance stabilized control signal intensity for each replicate of the plurality of peptides of the control dataset;
c. filtering the plurality of peptides to identify one or more peptides that are consistently phosphorylated or consistently unphosphorylated in the experimental dataset, optionally by examining replicate variability of variance stabilized signal intensities using a χ² test and/or subject variability (such as animal-animal variability) optionally using a F-test statistic;
d. identifying an overlapping set of peptides consistently phosphorylated or consistently unphosphorylated in the experimental dataset and the control dataset;
e. for the set of peptides consistently phosphorylated or consistently unphosphorylated in the experimental dataset and the control dataset, calculating a treatment variability value of the variability between the variance stabilized experimental signal intensity and the variance stabilized control signal intensity for each peptide, optionally using a one-sided t-test; and
f. identifying one or more peptides that is/are differentially phosphorylated in the experimental sample compared to the control sample.

In an embodiment, the method comprises comparing multiple treatments and/or subjects. Wherein multiple treatments are employed, they can be all compared to a single control, or each treatment can be compared to specific control. In an embodiment, where multiple treatments are to be compared, each experimental signal intensity of each peptide in the experimental datasets is subtracted for the signal intensity of a biological control signal intensity.

Identifying peptides that are consistently phosphorylated or consistently unphosphorylated and/or differentially phosphorylated can be used to identify proteins that are phosphorylated in response to a treatment. For example, the peptide on the peptide array may correspond to a specific protein and or group of related proteins. Identifying which peptides are phosphorylated indicates which proteins can be phosphorylated by a particular treatment or condition.

Peptides identified as differentially phosphorylated in an experimental dataset compared to a control or between experimental datasets, can be further subjected to further analysis including for example, to gene ontology enrichment analysis and/or signal transduction analysis. Accordingly, in an embodiment, the method further comprises generating a list of GO terms for consistently phosphorylated/unphosphorylated or differentially phosphorylated peptides, for example according to treatment. The GO terms can be further filtered to identify GO terms that repeated frequently.

As used herein “GO annotation” or “Gene Ontology annotation” refers to GO terms which is a controlled vocabulary of terms contributed by members of the GO consortium that have been assigned to gene products for classification of those products and describing gene product characteristics and gene product annotation data.

As another example, the identified peptides can be analysed to identify signaling pathways activated by a treatment. Accordingly, an aspect includes a method for identifying one or more cellular signaling pathways modulated in an experimental sample treated with a stressor compared to a control sample comprising:
a. identifying one or more peptides that are differentially phosphorylated in an experimental sample compared to a control sample according to a method described herein;
b. querying a database comprising gene ontology annotations and/or biological information for a plurality of proteins for one or more of the peptides identified as differentially phosphorylated; and
c. identifying one or more cellular pathways comprising the one or more peptides identified as differentially phosphorylated.

In another aspect, preprocessed data is further subjected to cluster analysis. Accordingly, in an embodiment, the method further comprises clustering the transformed signal intensities and/or clustering the one or more consistently phosphorylated or consistently unphosphorylated or differentially phosphorylated peptides.

Clustering analysis is optionally applied to the average of the transformed replicate signal intensities (e.g. for each peptide for each treatment and/or subject) which are optionally adjusted by subtracting the signal intensity of the biological control for each treatment and/or subject.

Another embodiment includes a method for comparing kinome data between a control sample and an experimental sample treated with a stressor, comprising:
a. obtaining an experimental dataset comprising an experimental phosphorylation signal intensity for a plurality of peptides, each peptide present in at least two replicates;
b. obtaining a control dataset comprising control phosphorylation signal intensities for a plurality of peptides each peptide present in at least two replicates;
c. transforming the phosphorylation signal intensity of each replicate of the plurality of peptides of
i. the experimental dataset using a variance stabilizing transformation to provide an experimental variance stabilized signal intensity for each replicate; and
ii. the control dataset using a variance stabilizing transformation to provide a control stabilized signal intensity for each replicate;
d. averaging the replicate experimental variance stabilized signal intensities for each peptide to obtain an average experimental intensity and averaging the replicate control variance stabilized signal intensities for each peptide to obtain an average control intensity; and
e. clustering the average replicate intensities optionally by hierarchical clustering or principal component analysis.

Clustering can optionally be employed to compare clusters of treatments, clusters of peptides or signaling pathways.

In embodiments wherein multiple treatments (e.g. stressors) are compared, the method can further comprise subtracting intensities of one or more biological controls from the experimental intensity and performing the cluster analysis on the subtracted treatment intensity.

In an embodiment, the peptides identified as differentially phosphorylated are clustered according to a subgroup of a treatment cluster based on GO annotations.

The stressor can be any agent that causes a biological response. For example, the stressor can comprise a biological agent, a physical agent, or a chemical agent. In an embodiment, the biological agent comprises an infectious agent or a macromolecule. In an embodiment, the infectious agent comprises a microorganism, such as a bacterial entity or fragment thereof, a viral entity or fragment thereof, or a fungal entity or fragment thereof, wherein the fragment is antigenic.

In an embodiment, the phosphorylation data is obtained by a contacting a sample with a known or unknown phenotype or one or more experimental cell populations each with a stressor, contacting a control cell population with a control treatment, lysing the cells to obtain an experimental sample and a control sample respectively, contacting the experimental sample with the experimental peptide array and contacting the control sample with the control peptide array, under conditions suitable for kinase phosphorylation. Conditions that are suitable for kinase phosphorylation are well known in the art and include for example incubation at a suitable temperature such as 37° C. for mammalian kinases, and providing an ATP source. Suitable conditions are for example described by Jalal et al. 2009 (37).

In an embodiment, the phosphorylated peptides are visualized by incubating the peptide array with a phosphospecific fluorescent stain, such as ProQ Diamond Phosphoprotein Stain (Invitrogen), and destaining.

In an embodiment, the conditions comprise providing a labeled phosphate ATP source (e.g labeled ATP and/or other suitable labeled ATP analog) that is a suitable substrate for kinase transfer; and acquiring phosphorylation signal intensities using for example a phosphoimager. In an embodiment, the labeled phosphate source comprises ATP wherein the terminal phosphate is labeled, optionally with a radioactive or fluorescent label. In an embodiment, the phosphorylation signal intensity comprises a radioactive signal.

The methods are useful for example for identifying novel biomarkers that are phosphorylated consistently or unphosphorylated consistently in a disease, condition or disorder or that are phosphorylated consistently or unphosphorylated consistently by a treatment.

As mentioned above, R package statistical programs can be used to calculate one or more of the values and/or transformations. In an embodiment, the signal intensity of each replicate is VSN transformed using the R package vsn.

In an embodiment, the phosphorylation consistency value comprises determining χ² statistic (TS₁). In an embodiment, the p-value is calculated using R package pchisq.

In certain embodiments, the method comprises comparing more than one sample or experimental sample. Wherein intersample variability may be confounding, inter-sample variability is determined by assessing whether there are significant differences among samples (e.g. corresponding to a subject) treated with a same stressor using a F-test statistic

TS₂=MS_{B}/MS_{W}

wherein MS_{B} is a mean squared between subjects and wherein MS_{W} is a Mean Squared Within Subjects and each are calculated.

In an embodiment, the one or more peptides that is/are differentially phosphorylated in the experimental sample compared to the control sample, or compared to a second experimental sample is identified using a one-sided paired t-test (alternatively referred to as a “paired t-test” herein), wherein the t-test statistic is calculated.

### Wherein

p-value=P[TS₃>t(n−1)](phosphorylation)

p-value=P[TS₃<−t(n−1)](dephosphorylation)

wherein peptides with a p-value less than a selected threshold are differentially phosphorylated.

In an embodiment, the one-sided paired t-test is calculated using R package t.test with paired=True.

In an embodiment, the method further comprises querying a database comprising protein annotations comprising descriptive terms associated with a catalogue of proteins, optionally gene ontology (GO) terms, optionally wherein the query comprises inputting a protein identifier for a protein comprising a peptide selected from the peptides identified as differentially phosphorylated, optionally an accession number such as a UniProt accession number or an Entrez Gene ID, and optionally generating a list of descriptive terms, optionally GO terms, for one or more of the plurality of peptides identified as differentially phosphorylated. In order to identify patterns and/or signaling pathways activated by a treatment, the frequency of each term for the one or more peptides phosphorylated or differentially phosphorylated is ranked according to frequency. The ranked list can be further filtered to identify common terms, for example descriptive terms that are identified for more than one of the peptides, such as descriptive terms that are identified with a selected frequency, for example at least 2 times, at least 3 times, at least 4 times, at least 5 times or more depending for example on the number of peptides being queried.

In another embodiment, the method comprises querying a database comprising signaling pathway annotations for a signaling pathway associated with a protein comprising a peptide selected from the peptides identified as differentially phosphorylated, optionally querying a KEGG or InnateDB database, optionally wherein the query comprises inputting a protein identifier for the protein comprising the peptide, optionally an accession number such as a UniProt accession number or an Entrez Gene ID, and optionally generating a list of one or more signaling pathways for one or more of the plurality of peptides.

As mentioned, the identified peptides can be clustered. In an embodiment, the one or more peptides consistently phosphorylated are clustered by a hierarchical clustering method and/or a principal component analysis (PCA) to cluster the one or more peptides according to treatment and/or subject-treatment combinations. In an embodiment, the hierarchical clustering method comprises considering each subject/treatment combination as a cluster with a single element; identifying two most similar clusters and merging the two most similar clusters; and iteratively calculating a distance between remaining clusters and the merged cluster to cluster the one or more peptides consistently phosphorylated. In another embodiment, the hierarchical clustering method comprises a clustering method and a distance measurement optionally “Average Linkage+(1-Pearson Correlation)”, “Complete Linkage+Euclidean Distance”, and “McQuitty+(1-Person Correlation)”. In yet a further embodiment, the hierarchical clustering is performed using R package heatmap.2 from the glpots package. In another embodiment, the PCA is performed using R program prcomp from the stats package.

As described herein, the preprocessing step uses a variance stabilizing module to bring negative and positive signals (after background corrections) onto the same positive scale while maintaining their correlations and minimizing the mean-variance dependence issue. Given the nature of the kinome data, this is not sufficiently dealt with by the typical normalization techniques in popular software such as GeneSpring or the limma package from Bioconductor. Because of the stabilization of variance in the data, the present method allows use of more standard statistical tests such as t-tests and F-tests. Consequently, spot-spot and subject-subject variation are rigorously considered to take into account both the technical and biological variation, which are more of a concern in kinome analysis than in conventional gene expression analysis. The paired t-test allows more peptides to be taken into consideration in the pathway analysis. Other multiple hypothesis testing such as Bonferroni and moderated t-test from limma have proven over-stringent in kinome analysis. Relevant databases are probed for known signaling pathways using the identified differentially phosphorylated peptides. In addition, Gene Ontology enrichment and clustering analysis are used to draw further insights from the data.

As used in this specification and the appended claims, the singular forms “a”, “an” and “the” include plural references unless the content clearly dictates otherwise. Thus for example, a composition containing “a compound” includes a mixture of two or more compounds. It should also be noted that the term “or” is generally employed in its sense including “and/or” unless the content clearly dictates otherwise.

In understanding the scope of the present disclosure, the term “comprising” and its derivatives, as used herein, are intended to be open ended terms that specify the presence of the stated features, elements, components, groups, integers, and/or steps, but do not exclude the presence of other unstated features, elements, components, groups, integers and/or steps. The foregoing also applies to words having similar meanings such as the terms, “including”, “having” and their derivatives. Finally, terms of degree such as “substantially”, “about” and “approximately” as used herein mean a reasonable amount of deviation of the modified term such that the end result is not significantly changed. These terms of degree should be construed as including a deviation of at least ±5% of the modified term if this deviation would not negate the meaning of the word it modifies.

In understanding the scope of the present disclosure, the term “consisting” and its derivatives, as used herein, are intended to be close ended terms that specify the presence of stated features, elements, components, groups, integers, and/or steps, and also exclude the presence of other unstated features, elements, components, groups, integers and/or steps.

The recitation of numerical ranges by endpoints herein includes all numbers and fractions subsumed within that range (e.g. 1 to 5 includes 1, 1.5, 2, 2.75, 3, 3.90, 4, and 5).

It is also to be understood that all numbers and fractions thereof are presumed to be modified by the term “about.” Further, it is to be understood that “a,” “an,” and “the” include plural referents unless the content clearly dictates otherwise. The term “about” means plus or minus 0.1 to 50%, 5-50%, or 10-40%, preferably 10-20%, more preferably 10% or 15%, of the number to which reference is being made.

Further, the definitions and embodiments described in particular sections are intended to be applicable to other embodiments herein described for which they are suitable as would be understood by a person skilled in the art. For example, in the following passages, different aspects of the invention are defined in more detail. Each aspect so defined may be combined with any other aspect or aspects unless clearly indicated to the contrary. In particular, any feature indicated as being preferred or advantageous may be combined with any other feature or features indicated as being preferred or advantageous.

The following non-limiting examples are illustrative of the present disclosure:

### EXAMPLES

### Example 1

The biological samples were collected from a S88 colony selected for testing. The varroa sensitive line G4 was selected from a Meadow Ridge apiary from a cross made previously. The colony selection was made by testing for varroa on adult bees by the alcohol wash method.

A bee specific peptide array was designed with 300 possible phosphorylation sites (e.g. peptides listed in Table 1, including some duplicates). This array was validated by examining honey bee head and thorax extracts in control samples and analysis of two extreme phenotypes for varroa tolerance was initiated. The results of these informative investigations are below.

### Peptide Arrays

The identification of peptides for inclusion on the Bee Peptide Array was performed using DAPPLE described in Example 4 and in U.S. 61/537,941, filed Sep. 22, 2011 herein incorporated by reference in its entirety.

All publicly available phosphorylation databases including drosophila were used to select the peptides.

Peptides identified, which are listed in Table 1, were used to construct an array for bee kinome analysis.

Design, construction and application of the peptide arrays is based upon a previously reported protocol with modifications (37).

Briefly, the peptides were spotted in a grid pattern on a block. Each block contains 298 test peptides, two negative control peptides, and seven positive control proteins. Examples of negative control or negative reference peptides are peptides that would not contain any Ser, Thr or Tyr residues. Positive control peptides could include for example histones 1 through 4, bovine myelin basic protein (MBP), and α/β casein.

Each array contains three replicate blocks in the same configuration. Each positive control is a full-length protein. These proteins are mainly included to aid in visualization and grid assignment of the blocks. In addition, to determine intraexperimental variability in substrate phosphorylation, each block of 300 peptides is printed in triplicate. The final physical dimensions of the arrays are 19.5 mm by 19.5 mm, with each peptide spot having a diameter of ˜350 μm and separated by 750 μm.

Notably the kinome experiments for all the animals were performed simultaneously in a single run minimizing the possibility of technical variances in the analysis.

Briefly, for test samples a whole frozen bee was ground up using mechanical force, pelleted and lysed by addition of 100 μL lysis buffer (20 mM Tris-HCL pH 7.5, 150 mM NaCl, 1 mM EDTA, 1 mM EGTA, 1% Triton, 2.5 mM sodium pyrophosphate, 1 mM Na₃VO₄, 1 mM NaF, 1 μg/mL leupeptin, 1 g/mL aprotinin, 1 mM PMSF) (all products are from Sigma Aldrich unless indicated otherwise). Cells were incubated on ice for 10 minutes and spun in a microcentrifuge for 10 minutes at 4° C. A 70 μl aliquot of this supernatant was mixed with 10 μl of activation mix (50% Glycerol, 500 μM ATP (New England Biolabs, Pickering, ON), 60 mM MgCl₂, 0.05% v/v Brij-35, 0.25 mg/mL BSA) and incubated on the array for 2 hours at 37° C. Arrays were then washed with PBS-(1%) Triton.

Slides were submerged in phospho-specific fluorescent ProQ Diamond Phosphoprotein Stain (Invitrogen) with agitation for 1 hour. Arrays were then washed three times in destain containing 20% acetonitrile (EMD Biosciences, VWR distributor, Mississauga, ON) and 50 mM sodium acetate (Sigma) at pH 4.0 for 10 minutes. A final wash of the arrays was done with distilled deionized H₂O. Arrays were air dried for 20 min then centrifuged at 300×g for 2 minutes to remove any remaining moisture from the array. Arrays were read using a GenePix Professional 4200A microarray scanner (MDS Analytical Technologies, Toronto, ON) at 532-560 nm with a 580 nm filter to detect dye fluorescence. Images were collected using the GenePix 6.0 software (MDS) and the spot intensity signal collected as the mean of pixel intensity using local feature background intensity background calculation with the default scanner saturation level.

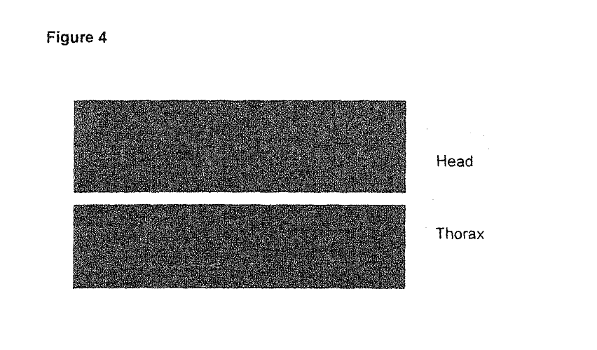

The bee specific peptide array comprising 300 peptides was validated examining head and thorax samples of bee larvae (FIG. 4).

### Varroa Sensitivity

Varroa mite infection rates for varroa sensitive (G4) and varroa resistant (S88) honey bees were assessed over time.

### Test Conditions

Varroa sensitive (G4) and varroa resistant (S88) honey bees were profiled for phosphorylation patterns using the constructed array. In a second experiment, varroa mite infected G4 and S88 were used for sampling. Three bees per group were assessed.

### Array Analysis

The array was analysed using the method described in Example 3 below and in PCT/CA2011/000764, filed Jun. 30, 2011 herein incorporated by reference in its entirety.

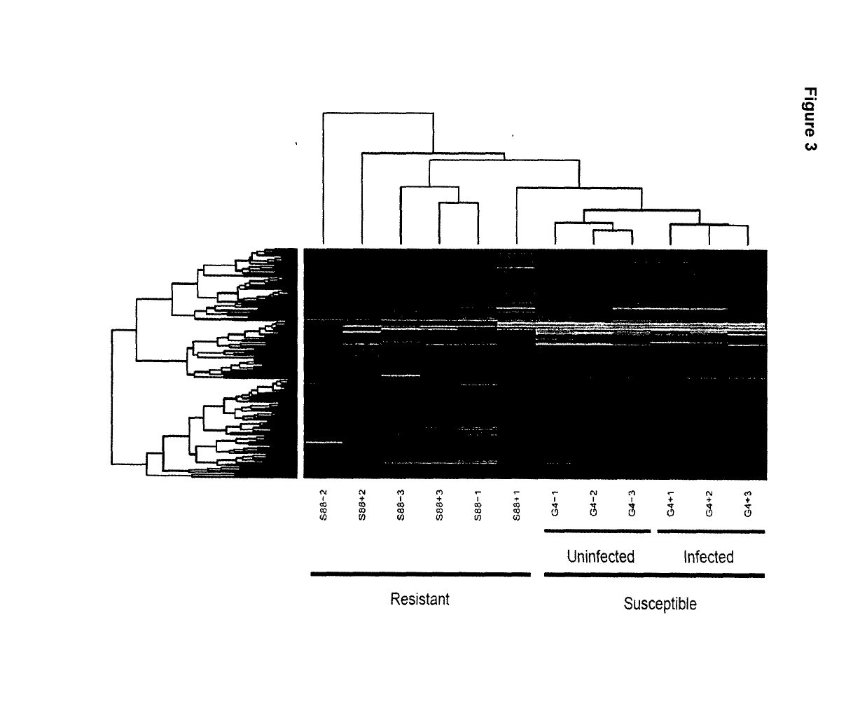

The kinome data sets were subjected to hierarchical clustering analysis. “Average Linkage+(1−Pearson Correlation)” was used for clustering both the bee-treatments (in vertical direction) and the peptides (in horizontal direction) (FIG. 3). Each column represents the kinome activity of individual larvae (n=3/treatment). Larvae from two colonies (G4 and S88) were selected for either the presence (+) or absence (−) of Varroa mites. Cluster analysis segregated kinome profiles first by colony phenotype (S88: Resistant; G4: susceptible) and then segregated G4 larvae by response to Varroa infection.

### Results

FIG. 4 which shows the validation results using head and thorax samples of bee larvae. The lit areas on each slide for head and thorax are three replicate blocks of the 300 peptides. Each spot or dot within the array represents an individual peptide. Light spots represent phosphorylation events, dark spots represent a lack of kinase activity.

The results revealed excellent kinase activity, with strong signals for individual peptides within each array and differential kinase activity when comparing the head and thorax (FIG. 4).

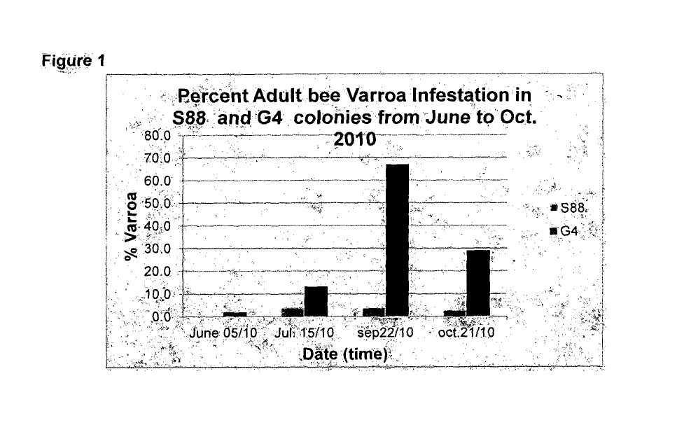

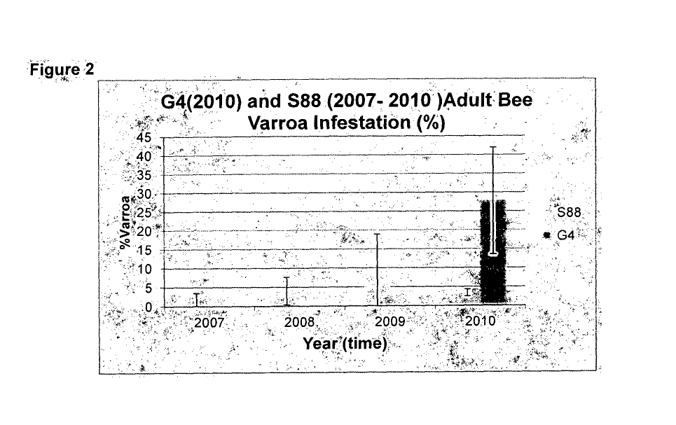

The S88 varroa tolerant phenotype never showed adult varroa infestations over 18% between 2007 and 2010 (FIG. 2). The sensitive phenotype (G4) had adult varroa levels increase from less than 1% to 67% in 88 days (FIG. 1). In the tolerant colony (S88) the increase was 1.8% in the same time period (FIG. 1). Varroa mite levels were less than 1% in both colonies at establishment.

Kinome array analyses of varroa sensitive (G4) and tolerant (S88) honey bee colonies in the presence and absence of varroa infestation, is shown in FIG. 3. The kinome cluster analyses clearly separated the two extreme phenotypes described in FIGS. 1 and 2. The arrays also showed a distinct difference in cellular responses to varroa infection in the two phenotypes.

These results show how application of kinome array analyses can clearly discriminate between honey bee phenotypes showing tolerance or sensitivity to varroa infection. Kinome analyses should therefore be effective at identifying and selecting many different honey bee phenotypes. These results suggest phenotyping capability of data generated by kinome analyses should be generally applicable in many different species.

A list of the peptides that were differentially phosphorylated in G4 and S88 bees in both infected and uninfected samples is provided in Table 2

Table 3 provides the phosphorylation level of peptides in infected G4 (susceptible bees) vs. infected S88 (tolerant) bees.

Table 4 provides the phosphorylation level of peptides in uninfected G4 (susceptible) vs. uninfected S88 (tolerant bees).

### Example 2

The method described in Example 1 is used to identify a profile for other phenotypes in other organisms.

Bees identified as having one or more desirable phenotypes are used for breeding to obtain lines with the desirable phenotype or phenotypes.

### Example 3

A set of statistical tests is used to address the variability issues existing between technical replicates and between biological replicates when identifying true differential peptides specific to a treatment under investigation while eliminating misleading factors that interfere with the interpretations of the results. Clustering analyses such as hierarchical clustering and principal component analysis (PCA) are incorporated into the workflow for comparative visualization of kinome patterns from the cells under various treatments.

The framework has been implemented primarily in the language R (39) facilitated by some PERL and BASH scripts.

### 2. Methods

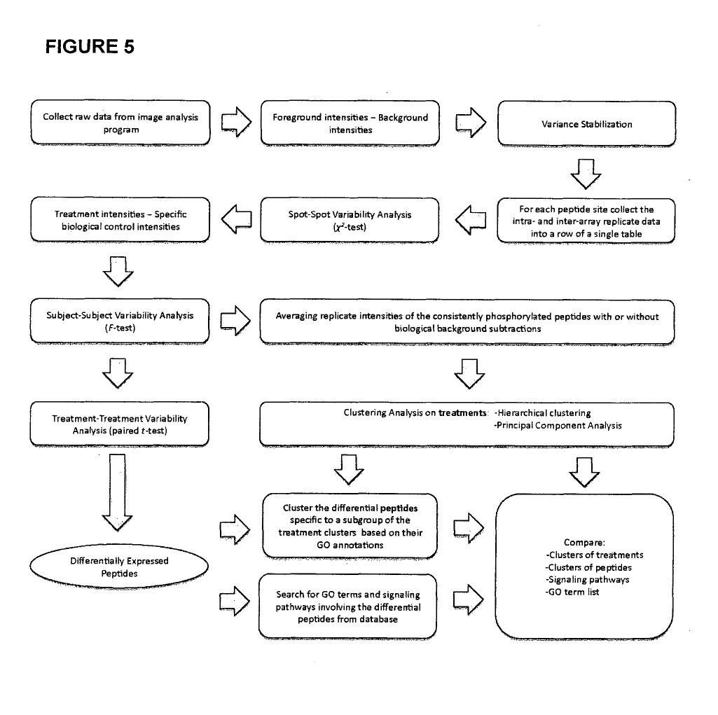

A general workflow of the following analytical steps is outlined in FIG. 5. All the calculations below can be done by R console unless noted otherwise (39). Specific R packages used are mentioned wherever applied. All the R packages used are publicly available from: www.R-project.org and www.bioconductor.org (121).

### 2.1 Data Preprocessing

In all datasets, the specific responses of each peptide are calculated by subtracting background intensity from foreground intensity.

The resulting data is transformed using a variance stabilization (VSN) model (38). The transformation brings all the data onto the same scale while alleviating variance-mean dependence. Only for the subsequent clustering analysis, is the average for each of the peptides in a single treatment taken over the transformed replicate intensities. If applicable, the intensities induced by the treatments are adjusted by subtracting the intensities of the biological control of the same subject. R package vsn can be used for the VSN transformation (59).

### 2.2 Spot-Spot Variability Analysis (Replicate Variability)

Chi-squared (χ²) test is used to examine the variability among the spots corresponding to the same treatment (53). Formally, the null hypothesis H₀ claims that there is no difference among intensities from the replicate spots, and alternative hypothesis H_{A} states that there exists significant variation among the replicates. The χ² test statistic (TS₁) is:

$${TS}_{1} = \frac{( n - 1 ) {s}^{2}}{{σ}^{2}}$$

where n is the number of replicates for each peptide in the treatment,

s²=1/nΣᵢ₌₁ⁿ(yᵢ− y̅)²

is the sample variance of the replicates for each peptide in a treatment,

\hat{σ}²=1/MΣⱼ₌₁^{M}sⱼ²

is the mean of all the variances for the replicates of the M peptides in the treatment (i.e., total number of distinct peptides included in an array), and

p-value=P[TS₁>χ²(n−1)]

Under the same treatment condition, the peptides with p-value less than a threshold are considered inconsistently phosphorylated or inconsistently unphosphorylated across the spots and will be eliminated from the subsequent clustering analyses. A strict confidence level (say, 0.01) can be used so that as much data as possible is retained. The p-value can be calculated using R program pchisq from the stats package.

### 2.3 Subject-Subject Variability Analysis

This step is done after biological background subtractions (if applicable) and only applied to datasets, where there is a concern of animal variation. For each of the peptides, an F-test is used to determine whether there are significant differences among the subjects under the same treatment condition (40).

Formally, let a be the number of subjects, n the number of intraarray replicates, N the total number of replicates for each peptide for each treatment, μᵢ the mean response of each peptide in the i^{th} subject for each treatment, and m the m^{th} replicate of a peptide in the i^{th} subject for each treatment. The null hypothesis H₀ claims that μ₁=μ₂= . . . =μₐ, or the mean phosphorylation intensities elicited by the identical peptide among the subjects are the same, and alternative hypothesis H_{A} states that not all subject means are equal. The F-statistic (TS₂) is calculated as:

$${TS}_{2} = \frac{{MS}_{B}}{{MS}_{W}}$$

$$where , \\ {MS}_{B} = \frac{{SS}_{B}}{{df}_{B}} = \frac{{\sum }_{i = 1}^{a} {( {\overline{y}}_{i} - \overline{y} )}^{2}}{a - 1}$$

$$( Mean Squared Between Subjects )$$

$${MS}_{W} = \frac{{SS}_{W}}{{df}_{W}} = \frac{{\sum }_{i = 1}^{a} {\sum }_{m = 1}^{n} {( {y}_{im} - {\overline{y}}_{i} )}^{2}}{N - a}$$

$$( Mean Squared Within Subjects )$$

where y̅ᵢ≡\hat{μ}ᵢ is the sample mean for i^{th} subject, y̅≡\hat{μ} the grand mean of all the subjects, and yᵢₘ the individual response of the m^{th} replicate in the i^{th} subject. Finally,

p-value=P[TS₂>F(a−1,N−a)]

Under the same treatment condition, the peptides with p-value less than a threshold are considered inconsistently expressed among the subjects and will be eliminated from the subsequent analyses. A strict confidence level (say, 0.01) can be used so that as much data as possible was retained.

### 2.4 Treatment-Treatment Variability Analysis

All peptides identified by the F-tests as having consistent patterns of response to various treatments across the subjects are subjected to one-sided paired t-tests to compare their signal intensities under a treatment condition with those under control conditions (40). Formally, the t-test statistic (TS₃) is calculated as:

$${TS}_{3} = \frac{\overline{D}}{{S}_{D} / \sqrt{n}}$$

where D̅ is the mean of the differences between responses for the same peptides induced by two different treatments, S_{D} the standard deviation of the differences, and n the number of replicate differences for that peptide between each treatment and control.

Finally,

p-value=P[TS₃>t(n−1)](phosphorylation)

p-value=P[TS₃<−t(n−1)](dephosphorylation)

The peptides with p-value less than a threshold (say, 0.05) are considered as differentially regulated and will be used for the subsequent analyses. No adjustment (as in the multiple testings) to the p-value is made to retain as much data as possible. The paired t-test is used here because it takes into account the interdependence between the same peptides under treatment and control conditions. Also note that the t-test is able to account for the variability (in terms of S_{D}) among the replicates so that replicates with significant p-values from the χ² tests will automatically have insignificant p-values from the t-test. However, this does not apply to datasets with multiple subjects, because significant variation for the same peptide among the subjects under the same treatment condition might be biologically meaningful, and it may confound the analysis, if treating these peptides as if they came from the same source.

The paired t-test can be done using R built-in function t:test from the stats package with paired=True. The results are presented in pseudoimages.

The latter can be generated based on the p-values from the one-sided t-tests for phosphorylation or dephosphorylation of each peptide. The depths of the coloration in red and green are inversely related to the corresponding p-values. For example, if the p-value for phosphorylation is 0.0001, then the redness in percentage will be 100%×(1−0.001)=99.9%. The same rationale is applied to dephosphorylated peptides. Thus, the combined colour depths of red and green will give an accurate account for the phosphorylation status of each peptide in the microarray. In addition, each dot in the plot is partitioned into parts, each of which represents a different treatment from the datasets. Moreover, the dots are rearranged in such a way that, going downwards by column and from left to the right of the array, the consistently expressed peptides across treatments are presented first followed by the inconsistent ones. Within the consistently expressed peptides, the ones with the most significant p-values for phosphorylation/dephosphorylation on average over the treatments being compared are presented first followed by less significant ones. Similarly, the inconsistent ones with the largest differences between the p-values from the treatments are presented first followed by the ones with smaller differences. The original numberings for each peptide (i.e., the label below each circle) from the initial array layout are unchanged for indexing detailed information of the peptide. This representation of the results from differential analysis may facilitate the visualization process to identify conspicuous intensities of the peptides across treatments from various perspectives. The plots can be generated using R functions plot (for plotting the dots in different coordinates), rgb (for coloration), and polygon (for drawing half and ⅓ of the circle to represent each treatment in each partition of the circle).

### 2.5 Gene Ontology Enrichment Analysis

A complete list of the GO terms for all the peptides is generated from the GOTermFinder on-line server (go.princeton.edu/cgi-bin/GOTermFinder) based on their UniProt accession numbers from the Protein Knowledgebase (www.uniprot.org) (51). The GOTermFinder determines the significant GO terms using Bonferroni hypergeometric test. Briefly, the probability for annotating a GO term to a list of genes is assumed to have a hypergeometric distribution. The p-value for a GO term is calculated using the equation for the hypergeometric distribution taking into account the number of annotated genes with that GO term in the query list and in the genome database. The calculated p-value is then adjusted using a simulation technique. Specifically, if the number of the genes in the input data is n, then n genes are randomly sampled from a total gene pool from a selected database of the server. This random sampled gene population is used to calculate the p-value for a GO term the same way described above. The procedure is repeated 1000 times. The Bonferroni adjusted p-value for a GO term is determined as the fraction of the 1000 tests that produce p-values better than the p-value calculated for that GO term using the input gene list (51). Based on the nature of the studies, the GO terms provided by GOTermFinder can be further reduced. Using this reference list, the GO terms for each significantly phosphorylated or dephosphorylated peptide identified by the paired t-tests above in every treatment are obtained. The number of times each GO term appears for all the selected peptides is recorded. The GO terms that appear more than 5 times under all the treatments are captured as the common GO terms, and their descriptions become the column names for the output table. The remaining GO terms' descriptions are organized into a single column named “Others”. From column 3 downstream, each cell entry corresponds to a single GO term and a peptide. If the peptide is found to belong to the GO term category, the cell is filled with “1”; “0” otherwise. The encoding was done for the peptides that were found to be significantly phosphorylated or dephosphorylated exclusively or non-exclusively in a single treatment.

2.6 Probing Signaling Transduction Pathways from Database

The identifiers such as GeneSymbols corresponding to the differential peptides detected in each treatment can be used to probe database such as KEGG (www.genome.jp/kegg/tool/search_pathway.html) or InnateDB (www.innatedb.com) to discover known signaling pathways that are specifically induced by the treatment under investigation (60; 61; 46; 62).

### 2.7 Clustering Analysis

The preprocessed data is subjected to hierarchical clustering and principal component analysis (PCA) to cluster peptide response profiles across treatments or subject-treatment combinations. For hierarchical clustering, three popular independent combinations of clustering method and distance measurement are recommended, namely “Average Linkage+(1−Pearson Correlation)”, “Complete Linkage+Euclidean Distance”, and “McQuitty+(1−Pearson Correlation)” (44; 43; 41; 42). In general, each subject/treatment vector is considered as a singleton (i.e., a cluster with a single element) at the initial stage of the clustering. The two most similar clusters are merged and the distances between the newly merged clusters and the remaining clusters are updated, iteratively. The calculations of similarity/distance between the clusters and the update step are algorithmically specific. The “Average Linkage+(1−Pearson Correlation)” is the method used by Eisen et al. (45). It takes the average over the merged (i.e., the most correlated) kinome profiles and updates the distances between the merged clusters and other clusters by recalculating the correlations between them. Formally, the Pearson correlation between any two vectors of subject/treatment of M peptides, say X and Y, is computed as

$${r}_{XY} = \frac{{\sum }_{i = 1}^{M} ( {x}_{i} - \overline{x} ) ( {y}_{i} - \overline{y} )}{\sqrt{{\sum }_{i = 1}^{M} {( {x}_{i} - \overline{x} )}^{2} {\sum }_{j = 1}^{M} {( {y}_{i} - \overline{y} )}^{2}}}$$

In “Complete Linkage+Euclidean Distance”, the distance between any two clusters is considered as the Euclidean distance between the two farthest data points in the two clusters (41; 42). Formally, the Euclidean distance between two subject/treatment vectors of M peptides, say X and Y, is calculated as:

dist(X,Y)=\sqrt{(x₁−y₁)²+(x₂−y₂)²+ . . . +(x_{M}−y_{M})²}

Finally, the McQuitty method updates the distance between the two clusters in such a way that upon merging clusters C_{X} and C_{Y} into a new cluster C_{XY}, the distance between C_{XY} and each of the remaining clusters, say C_{R}, is calculated taking into account the sizes of C_{X} and C_{Y} (43). Mathematically, let the size of C_{X} be n_{X} and size of C_{Y} be n_{Y}, then:

$$dist ( {C}_{XY} , {C}_{R} ) = \frac{{n}_{X} \times dist ( {C}_{X} , {C}_{R} ) + {n}_{Y} \times dist ( {C}_{X} , {C}_{R} )}{{n}_{X} + {n}_{Y}}$$

PCA is a variable reduction procedure. Basically, the calculation is done by a singular value decomposition of the centered and scaled data matrix (67). As a result, PCA transforms a number of possibly correlated variables into a smaller number of uncorrelated or orthogonal variables (i.e., principal components).

The first principal component accounts for the most variability in the data, and each succeeding component accounts for as much of the remaining variability as possible. Usually, the first three components account for larger than 50% of the variability in the data, and can be used as a set of the most important coordinates in a 3D plot to reveal the internal structure of the data.

R functions heatmap.2 from package gplots and prcomp from stats are used for hierarchical clusterings and PCA, respectively.

The 3D plot for the PCA using the first three principal components that account for the largest variability of the data is produced by R function scatterplot3d from package scatterplot3d.

### Example 4

DAPPLE (Design Array for PhosPhoryLation Experiments) is a collection of Perl scripts to easily, quickly, and accurately identify potential phosphorylation sites in an organism of interest.

### Methods

DAPPLE requires several input files: the proteome of the target organism (for which the user wants to design a kinome microarray) in FASTA format; the proteomes of the organisms represented in the database of phosphorylation sites, also in FASTA format; and the phosphorylation site data. If a particular organism represented in the phosphorylation site data does not have a proteome available, then the known phosphorylation sites from that organism can still be used; however, DAPPLE will be unable to output information for the “RBH?” column of the output table. The phosphorylation site data could be obtained from a number of sources, including the PhosphoSitePlus database (Hornbeck et al., 2004), Phospho.ELM (Diella et al., 2004, 2008), or the literature. This study used data from PhosphoSitePlus, which can be obtained from www.phosphosite.org/downloads/Phosphorylation site dataset.gz. As the PhosphoSitePlus data file contains entries with identical sequences (from different organisms), duplicate sequences are first removed. The sequences of the non-redundant phosphorylation sites are used as queries to the standalone version of blastp (ftp://ftp.ncbi.nlm.nih.gov/blast/executables/blast+/LATEST), with the target organism's proteome as the database. Unlike in Jalal et al. (2009) (37), the queries are not limited to those from human. The output from blastp is then parsed using the BioPerl (Stajich et al., 2002) module SearchIO, and the accession number and sequence of the best match, if any, for each query are saved. If there are multiple matches with the same E-value as the best match, then only the first result returned by BLAST is used. Additional information about the match is then saved or computed, and ultimately presented in the DAPPLE output table (described below).

Due to the short length of the query sequences (between eight and fifteen amino acids), the full protein corresponding to the best match may not be orthologous to the full protein corresponding to the query sequence. In Jalal et al. (2009), this problem was addressed by manually comparing the annotations of the proteins corresponding to the query and the match. However, this approach suffers from the drawbacks described in the introduction; thus, DAPPLE uses the well-established reciprocal BLAST hits (RBH) method to ascertain orthology (Moreno-Hagelsieb and Latimer, 2008). For a given known phosphorylation site X from organism A with best match Y in organism B (the target organism), let X′ be the full protein corresponding to X, and Y′ be the full protein corresponding to Y. DAPPLE will declare X′ and Y′ as orthologues if and only if Y′ is the best match when X′ is used as a query sequence and the proteome of organism B is used as the database, and X′ is the best match when Y′ is used as a query sequence and the proteome of organism A is used as the database. In this case, “the best match” is defined as any protein that has the smallest E-value. For instance, if X′ is not the first result returned by BLAST when Y′ is used as a query sequence and the proteome of organism A is used as the database, then X′ and Y′ can still be declared as orthologues if the E-value of the match against X′ is equal to that of the first result returned by BLAST.

The output of DAPPLE is a table in which each row represents the result of a BLAST search using, as a query, one of the known phosphorylation sites in the PhosphoSitePlus data file. The table is in a tab-delimited plain text format that can easily be subsequently manipulated. This table contains many columns. The following list describes each column, with X, Y, X′, and Y′ having the same meaning as above.
Query accession—the accession number of X′.
Query description—a description of X′.
Query organism—the organism that encodes X′.
Query sequence—the amino acid sequence of X.
Query site—the phosphorylated residue in X′; e.g. Y482.
Hit site—the residue in Y′ that corresponds to the query site.
Hit accession—the accession number of Y′.
Hit description—a description of Y′.
Hit sequence—the amino acid sequence of Y.
Sequence differences—the number of sequence differences between the entirety of X (not just the portion that matched in the BLAST local alignment) and Y. For instance, if X=ABCDEFGH and Y=CDEFG, then the number of sequence differences would be 3.
Non-conservative sequence differences—as above, except counting only the number of non-conservative sequence differences (those with a score less than or equal to zero in the BLOSUM62 matrix).
9-mer sequence differences—the number of sequence differences between the nine-residue region centred at the phosphorylated residue of X, and the nine-residue region centred at the corresponding residue in Y.
9-mer non-conservative sequence differences—as above, except counting only the number of non-conservative sequence differences.
Hit protein rank—This column will be 1 if the E-value between X′ and Y′ when a blastp search is performed using X′ as the query and the target proteome as the database is equal to the smallest E-value returned by this search, even if Y′ is not the first result returned. Otherwise, it will be the number corresponding to the order in which Y′ is returned by BLAST. For instance, if the best hit has an E-value of 10⁻³² and Y′ is the fifth result returned and has an associated E-value of 10⁻²⁴, then this column will be 5.
Hit protein E-value—the E-value of the match between X′ and Y′ when X′ is used as the query and the target organism is used as the database.
RBH?—either “yes” or “no”, depending on whether X′ and Y′ are reciprocal BLAST hits.
Low-throughput references—the number of references reporting the use of low-throughput biological techniques to study X.
High-throughput references—the number of references reporting the use of high-throughput biological techniques to study X.

The rows are listed in increasing order of sequence differences. Since the output table will contain thousands of possible phosphorylation sites, the user needs some method of filtering the table so that he or she can intelligently choose which peptides to include on the array. For example, the user may wish to view only rows where the number of low-throughput references is greater than two, or to eliminate rows where the “RBH?” column is “no”. DAPPLE's documentation describes a number of UNIX commands that can be used to filter the output table in these and other ways, further aiding the user in designing species-specific kinome microarrays.

While the present disclosure has been described with reference to what are presently considered to be the preferred examples, it is to be understood that the disclosure is not limited to the disclosed examples. To the contrary, the disclosure is intended to cover various modifications and equivalent arrangements included within the spirit and scope of the appended claims.

All publications, patents and patent applications are herein incorporated by reference in their entirety to the same extent as if each individual publication, patent or patent application was specifically and individually indicated to be incorporated by reference in its entirety.

TABLE 1
Array Peptides

| | | | | SEQ |
|---|---|---|---|---|
| Query | | | | ID |
| accession | Query description | Hit accession | Hit sequence | NO: |
| Q9Z2B5 | Eukaryotic translation initiation | XP_001123105 | NKIDDCNYAIKRIAL | 1 |
| | factor 2-alpha kinase 3 | | | |
| Q9Y314 | Nitric oxide synthase-interacting | XP_001120134 | LPSFWIPSKTPEAK | 2 |
| | protein | | | |
| Q9Y2U5 | Mitogen-activated protein kinase | XP_001122147 | KSLVGTPYWMSPE | 3 |
| | kinase kinase 2 | | | |
| Q9Y2H1 | Serine/threonine-protein kinase | XP_001120829 | QHAQKETEFLRLKR | 4 |
| | 38-like | | | |
| Q9Y243 | RAC-gamma serine/threonine- | XP_396874 | TYGRTTKTFCGTPEY | 5 |
| | protein kinase | | | |
| Q9WTK7 | Serine/threonine-protein | XP_623596 | PFQGDNIYKLYENIG | 6 |
| | kinase 11 | | | |
| Q9VXE5 | Serine/threonine-protein | XP_001122147 | RRKSLVGTPYWMSPE | 7 |
| | kinase PAK mbt | | | |
| Q9VXE5 | Serine/threonine-protein | XP_001122147 | QELPRRKSLVGTPYW | 8 |
| | kinase PAK mbt | | | |
| Q9UQM7 | CaM kinase II subunit alpha | NP_001128422 | LKGAILTTMLATRNF | 9 |
| Q9UPZ9 | Serine/threonine-protein kinase | XP_003251030 | IRSRPPYTDYVSTRW | 10 |
| | ICK | | | |
| Q9UHD2 | Serine/threonine-protein kinase | XP_396937 | QEDQQFVSLYGTEEY | 11 |
| | TBK1 | | | |
| Q9R1U5 | Serine/threonine-protein kinase | XP_397175 | PGERLSTWCGSPPY | 12 |
| | SIK1 | | | |
| Q9R1U5 | Serine/threonine-protein kinase | XP_397175 | LSTWCGSPPYAAPE | 13 |
| | SIK1 | | | |
| Q9P286 | Serine/threonine-protein kinase | XP_001122147 | HRDIKSDSILLTADG | 14 |
| | PAK 7 | | | |
| Q9P0L2 | Serine/threonine-protein kinase | GeneMark.hmm1613 | TPGNKLDTFCGSPPY | 15 |
| | MARK1 | | | |
| Q9NR97 | Toll-like receptor 8; | XP_396158 | LYDAFISYSHKD | 16 |
| | CD_antigen = CD288 | | | |
| Q9NQU5 | Serine/threonine-protein kinase | XP_396779 | KRKSFIGTPYWMAPE | 17 |
| | PAK 6 | | | |
| Q9H4A3 | Serine/threonine-protein kinase | XP_001121340 | KNRSFAKSVIGTPEF | 18 |
| | WNK1 | | | |
| Q9H063 | Repressor of RNA polymerase III | XP_624527 | PHDLQALSPPQTS | 19 |
| | transcription MAF1 homolog. | | | |
| Q9ESN9 | C-Jun-amino-terminal kinase- | XP_396524 | VMSEKVQSLAGSIY | 20 |
| | interacting protein 3 | | | |
| Q9ER34 | Aconitate hydratase, | XP_391994 | VAVGDENYGEGSSRE | 21 |
| | mitochondrial | | | |
| Q9BWW4 | Single-stranded DNA- | XP_623511 | AREKLALYVYEYLLH | 22 |
| | binding protein 3 | | | |
| Q99759 | Mitogen-activated protein | XP_001122147 | KSLVGTPYWMSPE | 3 |
| | kinase kinase kinase 3 | | | |
| Q99623 | Prohibitin-2 | XP_624330 | ALSQNPGYLKLRKIR | 23 |
| Q99459 | Cell division cycle 5-like | XP_624906 | TPNTILATPFRS | 24 |
| | protein | | | |
| Q99459 | Cell division cycle 5-like | XP_624906 | PLKGGLNTPLNNSDF | 25 |
| | protein | | | |
| Q96CW1 | AP-2 complex subunit mu | XP_391965 | AQITSQVTGQIGWRR | 26 |
| Q94527 | Nuclear factor NF-kappa-B | Q86DH7 | YIQLKRPSDGATSEP | 27 |
| | p110 subunit | | | |
| Q92918 | Mitogen-activated protein | XP_396779 | ATINKRKSFIGTPYW | 28 |
| | kinase kinase kinase | | | |
| | kinase 1 | | | |
| Q92918 | Mitogen-activated protein | XP_396779 | KRKSFIGTPYWMAPE | 17 |
| | kinase kinase kinase | | | |
| | kinase 1 | | | |
| Q92900 | Regulator of nonsense | XP_393330 | LSQPGLSQAELSQD | 29 |
| | transcripts 1 | | | |
| Q920L2 | Succinate dehydrogenase | XP_392269 | YKERIDEYDYAKPLE | 30 |
| | [ubiquinone] flavoprotein | | | |
| | subunit, mitochondrial | | | |
| Q91Y86 | Mitogen-activated protein | XP_392806 | DLDHERMSYLLYQML | 31 |
| | kinase 8 | | | |
| Q8WUM4 | Programmed cell death | XP_396117 | KKDNDFIYHERIPDI | 32 |
| | 6-interacting protein | | | |
| Q8NEB9 | Phosphatidylinositol | XP_001121579 | ENLDLKLTPYRVLAT | 33 |
| | 3-kinase catalytic | | | |
| | subunit type 3 | | | |
| Q8IVH8 | Mitogen-activated protein | XP_396779 | ATINKRKSFIGTPYW | 28 |
| | kinase kinase kinase | | | |
| | kinase 3 | | | |
| Q8C863 | E3 ubiquitin-protein | XP_395191 | IDHNTRTTQWEDPR | 34 |
| | ligase Itchy | | | |
| Q7TNL5 | Protein phosphatase 2A | XP_392477 | KPLLRRKSDLPQDTY | 35 |
| | B56 delta subunit | | | |
| Q7L9L4 | Mps one binder kinase | XP_393046 | FGSRSSKTFKPKKNI | 36 |
| | activator-like 1A | | | |
| Q7KZI7 | Serine/threonine-protein | XP_394194 | TPGNKLDTFCGSPPY | 15 |
| | kinase MARK2 | | | |
| Q78DX7 | Proto-oncogene tyrosine- | XP_394148 | FGLARDIYKNDYYRK | 37 |
| | protein kinase ROS | | | |
| Q6P9R2 | Serine/threonine-protein | XP_396480 | KDPTKRPTATELLKH | 38 |
| | kinase OSR1 | | | |
| Q62627 | PRKC apoptosis WT1 regulator | XP_001120635 | LREKRRSTGVVHLPS | 39 |
| | protein | | | |
| Q62120 | Tyrosine-protein kinase JAK2 | XP_623692 | GSLLTYLRKNTNT | 40 |
| Q62120 | Tyrosine-protein kinase JAK2 | XP_624960 | GIANIAISPTIIRKN | 41 |
| Q61083 | Mitogen-activated protein | XP_396603 | ERKKRYTVVGNP | 42 |
| | kinase kinase kinase 2 | | | |
| Q60876 | Eukaryotic translation | XP_001120078 | PNDYSSTPGGTLFS | 43 |
| | initiation factor 4E-binding | | | |
| | protein 1 | | | |
| Q60876 | Eukaryotic translation | XP_001120078 | GGTLFSTTPGGTRIV | 44 |
| | initiation factor 4E-binding | | | |
| | protein 1 | | | |
| Q5XHZ0 | Heat shock protein 75 kDa, | XP_623366 | NLGTIARSGSRAFIE | 45 |
| | mitochondrial; HSP 75 | | | |
| Q5VT25 | Serine/threonine-protein | XP_395596 | QSNVAVGTPDYISPE | 46 |
| | kinase MRCK alpha | | | |
| Q5SWU9 | Acetyl-CoA carboxylase 1 | XP_624665 | VRFVVMVTPEDLKAN | 47 |
| Q5SRQ6 | Casein kinase 2, beta | XP_624048 | ETKMSSSEEVSWIS | 48 |
| | polypeptide | | | |
| Q5S007 | Leucine-rich repeat serine/ | XP_003249358 | SPVIIVGTHYDISYE | 49 |
| | threonine-protein kinase 2 | | | |
| Q3LRT3 | Salt-inducible kinase 2 | XP_397175 | LSTWCGSPPYAAPE | 13 |
| Q32NB8 | CDP-diacylglycerol-- | XP_397318 | GANLSNDYFTNRQDR | 50 |
| | glycerol-3-phosphate 3- | | | |
| | phosphatidyltransferase, | | | |
| | mitochondrial | | | |
| Q2NL82 | Pre-rRNA-processing | XP_624169 | FPDEVDTPQDILAK | 51 |
| | protein TSR1 homolog | | | |
| Q29122 | Myosin-VI; Unconventional | XP_392805 | GGIKGTVIMVPLK | 52 |
| | myosin-6 | | | |
| Q28147 | Nuclear inhibitor of | XP_003250277 | LGLPETETELDNLTE | 53 |
| | protein phosphatase 1 | | | |
| Q17446 | Mitogen-activated | XP_395384 | TENEMTGYVATRWYR | 54 |
| | protein kinase pmk-1 | | | |
| Q16665 | Hypoxia-inducible | XP_392382 | TFLSKHSLSMKFTY | 55 |
| | factor 1-alpha | | | |
| Q16584 | Mitogen-activated protein | XP_395037 | LAREVYKTTRMSAAG | 56 |
| | kinase kinase kinase 11 | | | |
| Q16584 | Mitogen-activated protein | XP_395037 | YKTTRMSAAGTYAW | 57 |
| | kinase kinase kinase 11 | | | |
| Q16539 | Mitogen-activated protein | XP_395384 | TENEMTGYVATRWYR | 54 |
| | kinase 14 | | | |
| Q15831 | Serine/threonine-protein | XP_623596 | LLLALDGTLKISDFG | 58 |
| | kinase 11 | | | |
| Q15208 | Serine/threonine-protein | XP_001120829 | NRRALAYSTVGTPDY | 59 |
| | kinase 38 | | | |
| Q15208 | Serine/threonine-protein | XP_001120829 | DWVFINYTFKRFEGL | 60 |
| | kinase 38 | | | |
| Q15084 | Protein disulfide- | XP_395981 | EEEIDLSDIDLDE | 61 |
| | isomerase A6 | | | |
| Q15078 | Cyclin-dependent kinase | XP_394967 | MGTVLSFSPRDRRGS | 62 |
| | 5 activator 1 | | | |
| Q15019 | Septin-2 | XP_395643 | YPLPDCDSDEDEDYK | 63 |
| Q14721 | Potassium voltage-gated | XP_393546 | YWGVDELYLESCCQ | 64 |
| | channel subfamily B | | | |
| | member 1 | | | |
| Q14164 | Inhibitor of nuclear factor | XP_396937 | EDQQFVSLYGTEEY | 65 |
| | kappa-B kinase subunit | | | |
| | epsilon | | | |
| Q13976 | cGMP-dependent protein | Q8SSX4 | GRKTWTFCGTPEY | 66 |
| | kinase 1 | | | |
| Q13573 | SNW domain-containing | XP_623623 | KIPRGPPSPPAPVMH | 67 |
| | protein 1 | | | |
| Q13557 | Calcium/calmodulin-dependent | GeneMark.hmm17653 | SVVHRQETVDCLKKF | 68 |
| | protein kinase type II | | | |
| | subunit delta | | | |
| Q13526 | Peptidyl-prolyl cis-trans | XP_624205 | GWEKRLSRSTGQHY | 69 |
| | isomerase NIMA-interacting 1 | | | |
| Q13526 | Peptidyl-prolyl cis-trans | XP_624205 | SHLLVKHSGSRRPSS | 70 |
| | isomerase NIMA-interacting 1 | | | |
| Q13188 | Serine/threonine-protein | XP_393691 | IMRLRKKTLQEDEIA | 71 |
| | kinase 3 | | | |
| Q13164 | Mitogen-activated protein | XP_393029 | HAGFLTEYVATRWYR | 72 |
| | kinase 7 | | | |
| Q13153 | Serine/threonine-protein | XP_001119958 | ENPLRALYLIATNG | 73 |
| | kinase PAK 1 | | | |
| Q13153 | Serine/threonine-protein | XP_003251334 | QGASGTVYTAIETST | 74 |
| | kinase PAK 1 | | | |
| Q12972 | Nuclear inhibitor of protein | XP_003250277 | EPKKKKYAKEAWPG | 75 |
| | phosphatase 1 | | | |
| Q09137 | 5′-AMP-activated protein | XP_623371 | VDPMKRATIEDIKKH | 76 |
| | kinase catalytic subunit | | | |
| | alpha-2 | | | |
| Q06830 | Peroxiredoxin-1 | XP_003249289 | HLAWVNTPRKQGGL | 77 |
| Q06609 | DNA repair protein RAD51 | XP_624827 | ETRICKIYDSPCLPE | 78 |
| | homolog 1 | | | |
| Q06210 | Glucosamine--fructose-6- | NP_001128421 | VATRRGSPLLVGIK | 79 |
| | phosphate aminotransferase | | | |
| | [isomerizing] 1 | | | |
| Q06187 | Tyrosine-protein kinase BTK | XP_394126 | RYVLDDQYTSSGGTK | 80 |
| Q05397 | Focal adhesion kinase 1 | XP_001120873 | DRTNDKVYDCTTSVV | 81 |
| Q05397 | Focal adhesion kinase 1 | XP_001120873 | IVDEEGDYSTPATRD | 82 |
| Q04206 | Transcription factor p65 | XP_395180 | IQLKRPSDGALSEP | 83 |
| Q04206 | Transcription factor p65 | XP_624626 | RPSDGDCSEPVKFTY | 84 |
| Q03468 | DNA excision repair protein | XP_001120586 | GANRVVIYDPDWNPA | 85 |
| | ERCC-6 | | | |
| Q02790 | Peptidyl-prolyl cis-trans | XP_395748 | LAKEKKLYANMFDKF | 86 |
| | isomerase FKBP4 | | | |
| Q02750 | Dual specificity mitogen- | XP_393416 | VSGQLIDSMANSFVG | 87 |
| | activated protein kinase | | | |
| | kinase 1 | | | |
| Q02750 | Dual specificity mitogen- | XP_393416 | KICDFGVSGQLIDSM | 88 |
| | activated protein kinase | | | |
| | kinase 1 | | | |
| Q00610 | Clathrin heavy chain 1 | XP_623111 | LLIDEEDYQGLRTSI | 89 |
| Q00535 | Cyclin-dependent kinase 5 | NP_001161897 | EKIGEGTYGTVFKAK | 90 |
| P98177 | Forkhead box protein O4 | XP_001122804 | FRPRASSNASS | 91 |
| P97784 | Cryptochrome-1 | A4GKG5 | SLRKLNSRLFVIRG | 92 |
| P84243 | Histone H3.3 | XP_624499 | ATKAARKSAPSTGGV | 93 |
| P83916 | Chromobox protein homolog 1 | XP_393875 | GYSNEENTWEPEENL | 94 |
| P80192 | Mitogen-activated protein | XP_395037 | TRMSAAGTYAWMAPE | 95 |
| | kinase kinase kinase 9 | | | |
| P78371 | T-complex protein 1 subunit | XP_393300 | GSRVRVDSMAKIAEL | 96 |
| | beta | | | |
| P70170 | ATP-binding cassette sub- | XP_003249371 | HDLRSRLTIIPQDPV | 97 |
| | family C member 9 | | | |
| P68431 | Histone H3.1 | XP_001120132 | KQTARKSTGGKAPRK | 98 |
| P68400 | Casein kinase II subunit | XP_623397 | DWGLAEFYHPGQEYN | 99 |
| | alpha | | | |
| P68104 | Elongation factor 1-alpha 1 | P19039 | EMHHEALTEALPGDN | 100 |
| P67775 | Serine/threonine-protein | XP_623105 | EPHVTRRTPDYFL | 101 |
| | phosphatase 2A catalytic | | | |
| | subunit alpha isoform | | | |
| P63244 | Guanine nucleotide-binding | XP_392962 | LCFSPNRYWLCAAFG | 102 |
| | protein subunit beta-2- | | | |
| | like 1 | | | |
| P63104 | 14-3-3 protein zeta/delta | GeneMark.hmm4290 | LTLWTSDTQGDADEA | 103 |
| P63000 | Ras-related C3 botulinum | CAX86545 | YDRLRPLSYPQTDVF | 104 |
| | toxin substrate 1 | | | |
| P62898 | Cytochrome c, somatic | P00038 | GQAPGYSYTDANKGK | 105 |
| P62826 | GTP-binding nuclear | XP_393761 | DRKVKAKSIVFHRKK | 106 |
| | protein Ran | | | |
| P62805 | Histone H4 | XP_003251221 | RGGVKRISGLIYEET | 107 |
| P62158 | Calmodulin | XP_624247 | MARKMKDTDSEEEIR | 108 |
| P61020 | Ras-related protein Rab-5B | XP_003251474 | KELQRQASPSIVIAL | 109 |
| P59241 | Serine/threonine-protein | CBM40275 | APSSRRNTLCGTLDY | 110 |
| | kinase 6 | | | |
| P56524 | Histone deacetylase 4 | XP_391882 | FPLRKTASEPNL | 111 |
| P56480 | ATP synthase subunit beta, | XP_624156 | LGENTVRTIAMDGTE | 112 |
| | mitochondrial | | | |
| P55823 | Elongation factor 2 | XP_392691 | GETRFTDTRKDEQER | 113 |
| P55211 | Caspase-9 | XP_395697 | LRSRCGTNEDCKNL | 114 |
| P55072 | Transitional endoplasmic | XP_392892 | AMRFARRSVSDNDIR | 115 |
| | reticulum ATPase | | | |
| P54764 | Ephrin type-A receptor 4 | Q5D184 | SYVDPHTYEDPNQAV | 116 |
| P54762 | Ephrin type-B receptor 1 | Q5D184 | YVDPHTYEDPNQAV | 117 |
| P53778 | Mitogen-activated protein | XP_395384 | RPTENEMTGYVATRW | 118 |
| | kinase 12 | | | |
| P53778 | Mitogen-activated protein | XP_395384 | ENEMTGYVATRWYR | 119 |
| | kinase 12 | | | |
| P53667 | LIM domain kinase 1 | XP_396603 | ERKKRYTVVGNPYW | 120 |
| P53350 | Serine/threonine-protein | XP_396707 | HEGERKKTVCGTPNY | 121 |
| | kinase PLK1; Polo-like | | | |
| | kinase 1 | | | |
| P53350 | Serine/threonine-protein | XP_396707 | LELCRKRSMMELHKR | 122 |
| | kinase PLK1; Polo-like | | | |
| | kinase 1 | | | |
| P53350 | Serine/threonine-protein | XP_396707 | HEGERKKTVCGTPNY | 121 |
| | kinase PLK1; Polo-like | | | |
| | kinase 1 | | | |
| P53349 | Mitogen-activated protein | XP_623135 | GSLVGTLNYVAPE | 123 |
| | kinase kinase kinase 1 | | | |
| P52565 | Rho GDP-dissociation | CAY09675 | GKVARGSYSVSSLF | 124 |
| | inhibitor 1 | | | |
| P52333 | Tyrosine-protein kinase | XP_396649 | QVARGMEYLASRRCI | 125 |
| | JAK3 | | | |
| P61813 | Cytoplasmic tyrosine- | XP_394126 | RYVLDDQYTSSGGTK | 80 |
| | protein kinase BMX | | | |
| P51812 | Ribosomal protein S6 | XP_394955 | DSEFTCKTPKDSPGV | 126 |
| | kinase alpha-3 | | | |
| P51812 | Ribosomal protein S6 | XP_394955 | TCKTPKDSPGVPPSA | 127 |
| | kinase alpha-3 | | | |
| P51692 | Signal transducer and | XP_397181 | KDQAFSKYYTP | 128 |
| | activator of transcription | | | |
| | 5B | | | |
| P51617 | Interleukin-1 receptor- | CBM40275 | RRNTLCGTLDYLPPE | 129 |
| | associated kinase 1 | | | |
| P50750 | Cyclin-dependent kinase 9 | XP_396015 | NGQPNRYTNRVVTLW | 130 |
| P50613 | Cyclin-dependent kinase 7 | XP_395800 | GSPNRINTHQVVTRW | 131 |
| P50516 | V-type proton ATPase | XP_623495 | LPPKSKGTVTYIAP | 132 |
| | catalytic subunit A | | | |
| P49840 | Glycogen synthase kinase- | XP_392504 | KGEPNVSYICSRYYR | 133 |
| | 3 alpha | | | |
| P49459 | Ubiquitin-conjugating | XP_003249705 | LDEPNPNSPANSLAA | 134 |
| | enzyme E2 A | | | |
| P49327 | Fatty acid synthase; | GeneMark.hmm24113 | FSRLGVLSPDCRCKS | 135 |
| P49138 | MAP kinase-activated | XP_392769 | DTLQTPCYTPYY | 136 |
| | protein kinase 2 | | | |
| P49137 | MAP kinase-activated | XP_392769 | SNHGLAISPGMKKRI | 137 |
| | protein kinase 2 | | | |
| P49023 | Paxillin | GeneMark.hmm18481 | ELDDLMASLSEFK | 138 |
| P48729 | Casein kinase I isoform | XP_393612 | KISEKKMSTPVEVLC | 139 |
| | alpha | | | |
| P46460 | Vesicle-fusing ATPase | XP_001120201 | MNRLIKASSKVEVD | 140 |
| P45983 | Mitogen-activated | GeneMark.hmm14772 | TTFMMTPYVVTRYYR | 141 |
| | protein kinase 8 | | | |
| P42345 | Serine/threonine-protein | CAZ78097 | IKRLHVSASNLQKAW | 142 |
| | kinase mTOR | | | |
| P41743 | Protein kinase C iota | XP_397273 | REGDTTATFCGTPNY | 143 |
| | type | | | |
| P41240 | Tyrosine-protein kinase | XP_393399 | ALKQNKFSNKSDMWS | 144 |
| | CSK | | | |
| P40926 | Malate dehydrogenase, | XP_392478 | SATLSMAYAGARFGF | 145 |
| | mitochondrial; Flags: | | | |
| | Precursor. | | | |
| P40429 | 60S ribosomal protein | XP_623813 | PFHFRAPSKILWKTV | 146 |
| | L13a | | | |
| P38919 | Eukaryotic initiation | XP_393356 | GQHVVSGTPGRVFDM | 147 |
| | factor 4A-III | | | |
| P38646 | Stress-70 protein, | NP_001153520 | VIGIDLGTTFSCVAV | 148 |
| | mitochondrial | | | |
| P37173 | TGF-beta receptor | XP_395928 | GQVGTRRYMAPEVLE | 149 |
| | type-2 | | | |
| P37040 | NADPH--cytochrome | XP_001119949 | SYRTALTHYLDITSNP | 150 |
| | P450 reductase | | | |
| P36897 | TGF-beta receptor | XP_003251656 | MTTSGSGSGLPLLVQ | 151 |
| | type-1 | | | |
| P35465 | Serine/threonine-protein | XP_001119958 | PTNFEHTVHVGFDA | 152 |
| | kinase PAK 1 | | | |
| P35234 | Tyrosine-protein phosphatase | XP_625071 | GLLERRGSSASLTIE | 153 |
| | non-receptor type 5 | | | |
| P35222 | Catenin beta-1 | NP_001172034 | QEYKKRLSMELTNSL | 154 |
| P35222 | Catenin beta-1 | NP_001172034 | RNEGVATYAAAVLFR | 155 |
| P34947 | G protein-coupled receptor | XP_394109 | LDIEQFSTVKGVNLD | 156 |
| | kinase 5 | | | |
| P33535 | Mu-type opioid receptor | GeneMark.hmm15186 | MQTVTNMYIVNLAIA | 157 |
| P32248 | C-C chemokine receptor | XP_396348 | ILHLMCISVDRYWAI | 158 |
| | type 7 | | | |
| P31749 | RAC-alpha serine/threonine- | XP_396874 | HFPQFSYQESHSA | 159 |
| | protein kinase | | | |
| P31749 | RAC-alpha serine/threonine- | XP_396874 | EVLEDNDYGRAVDWW | 160 |
| | protein kinase | | | |
| P31645 | Sodium-dependent serotonin | XP_624619 | SLWKGISTSGKVVW | 161 |
| | transporter | | | |
| P30050 | 60S ribosomal protein L12 | XP_623110 | KIGPLGLSPKKVGDD | 162 |
| P29992 | Guanine nucleotide-binding | XP_003250127 | RRREYQLTDSAKYYL | 163 |
| | protein subunit alpha-11 | | | |
| P29804 | Pyruvate dehydrogenase E1 | XP_623502 | SMSDPGTSYRTREEI | 164 |
| | component subunit alpha, | | | |
| | somatic form, mitochondrial | | | |
| P29804 | Pyruvate dehydrogenase E1 | XP_623502 | NGYGMGTSVDRASAS | 165 |
| | component subunit alpha, | | | |
| | somatic form, mitochondrial | | | |
| P29804 | Pyruvate dehydrogenase E1 | XP_003251259 | TYRYYGHSMSDPGTS | 166 |
| | component subunit alpha, | | | |
| | somatic form, mitochondrial | | | |
| P29476 | Nitric oxide synthase, brain | Q5FAN1 | IARAVKFTSKLFGRA | 167 |
| P29320 | Ephrin type-A receptor 3 | Q5D184 | ESATEGAYTTRGGKI | 168 |
| P29317 | Ephrin type-A receptor 2 | Q5D184 | SYVDPHTYEDPNQAV | 116 |
| P28482 | Mitogen-activated protein | XP_393029 | LGVLGSPSPEDLECI | 169 |
| | kinase 1 | | | |
| P28482 | Mitogen-activated protein | XP_393029 | HILGVLGSPSPEDL | 170 |
| | kinase 1 | | | |
| P28329 | Choline O-acetyltransferase | XP_392463 | VATYESAGIRRFALG | 171 |
| P28028 | Serine/threonine-protein | XP_396892 | LGQQDRSSSAPNV | 172 |
| | kinase B-raf | | | |
| P27448 | MAP/microtubule affinity- | GeneMark.hmm1613 | TPGNKLDTFCGSPPY | 15 |
| | regulating kinase 3 | | | |
| P27361 | Mitogen-activated protein | XP_393029 | APEIMLNSKGYTKSI | 173 |
| | kinase 3 | | | |
| P27361 | Mitogen-activated protein | XP_393029 | FLTEYVATRWYRAPE | 174 |
| | kinase 3 | | | |
| P26267 | Pyruvate dehydrogenase E1 | XP_003251259 | SMSDPGTSYRTREEV | 175 |
| | component subunit alpha | | | |
| | type I, mitochondrial | | | |
| P26038 | Moesin | XP_396252 | GRDKYKTLREIRKG | 176 |
| P25206 | DNA replication licensing | XP_625020 | SFGNKHVTPRTLTS | 177 |
| | factor MCM3 | | | |
| P25098 | Beta-adrenergic receptor | XP_396647 | AVLADVSYLMAMEKS | 178 |
| | kinase 1 | | | |
| P24941 | Cyclin-dependent kinase 2 | XP_393450 | EKIGEGTYGVVYKAK | 179 |
| P24928 | DNA-directed RNA polymerase | XP_623281 | SPNYSPTSPTYSPTS | 180 |
| | II subunit RPB1 | | | |
| P23572 | Cyclin-dependent kinase 1 | XP_393093 | FGIPVRVYTHEVVTL | 181 |
| P23443 | Ribosomal protein S6 kinase | XP_395876 | NRVFQGFTYVAPSIL | 182 |
| | beta-1 | | | |
| P23443 | Ribosomal protein S6 kinase | XP_395876 | QDGTVTHTFCGTIEY | 183 |
| | beta-1 | | | |
| P23437 | Cyclin-dependent kinase 2 | XP_393450 | GVPVRTYTHEIVTLW | 184 |
| P23396 | 40S ribosomal protein S3 | XP_623731 | SGVEVRVTPHRTEII | 185 |
| P22681 | E3 ubiquitin-protein ligase | XP_395448 | TAEQYELYCEMGSTF | 186 |
| | CBL | | | |
| P22288 | GTP cyclohydrolase 1 | XP_624456 | VKDIEMFSMCEHHLV | 187 |
| P21575 | Dynamin-1 | XP_394399 | NPEGRNVYKDYKQLE | 188 |
| P21399 | Cytoplasmic aconitate | XP_392993 | KEFNSYGARRGNDDV | 189 |
| | hydratase | | | |
| P19838 | Nuclear factor NF-kappa-B | Q86DH6 | KALRFRYECEGRS | 190 |
| | p105 subunit | | | |
| P18669 | Phosphoglycerate mutase 1 | XP_625114 | VQIWRRSFDTPPPPM | 191 |
| P17742 | Peptidyl-prolyl cis-trans | XP_393381 | KGFGYKGSSFHRVIP | 192 |
| | isomerase A | | | |
| P17612 | cAMP-dependent protein | CAC00652 | RVQGRTWTLCGTPEY | 193 |
| | kinase catalytic subunit | | | |
| | alpha | | | |
| P17220 | Proteasome subunit alpha | XP_393294 | VAMLMQEYTQSGGVR | 194 |
| | type-2 | | | |
| P16951 | Cyclic AMP-dependent | XP_003249317 | ADQTPTPTRFIRNCE | 195 |
| | transcription factor ATF-2 | | | |
| P16858 | Glyceraldehyde-3-phosphate | XP_393605 | ISWYDNEYGYSCRVI | 196 |
| | dehydrogenase | | | |
| P15172 | Myoblast determination | XP_001120527 | VDRRKAATLRERRRL | 197 |
| | protein 1 | | | |
| P15056 | Serine/threonine-protein | XP_396892 | FGLATAKTRWSGSQQ | 198 |
| | kinase B-raf | | | |
| P15056 | Serine/threonine-protein | XP_396892 | IGDFGLATAKTRWSG | 199 |
| | kinase B-raf | | | |
| P14618 | Pyruvate kinase isozymes | XP_624390 | FSHGTHEYHAETIAN | 200 |
| | M1/M2; | | | |
| P13639 | Elongation factor 2 | XP_392691 | KVMKFSVSPVVRVAV | 201 |
| P11960 | 2-oxoisovalerate dehydrogenase | XP_396003 | TYRIGHHSTSDDST | 202 |
| | subunit alpha, mitochondrial | | | |
| P11831 | Serum response factor | XP_001120126 | DNKLRRYTTFSKRKT | 203 |
| P11831 | Serum response factor | XP_001120126 | LRRYTTFSKRKTGIM | 204 |
| P11802 | Cyclin-dependent kinase 4 | XP_391955 | YDFEMRLTSVVVTQW | 205 |
| P11499 | Heat shock protein HSP | C1JYH6 | QEEYGEFYKSLTNDW | 206 |
| | 90-beta | | | |
| P11413 | Glucose-6-phosphate 1- | XP_001121185 | DLTYGSRYKDLKLPD | 207 |
| | dehydrogenase | | | |
| P11217 | Glycogen phosphorylase, | XP_623386 | QEKRKQISVRGIVDV | 208 |
| | muscle form | | | |
| P11021 | 78 kDa glucose-regulated | NP_001153524 | VFDLGGGTFDVSLLT | 209 |
| | protein | | | |
| P10860 | Glutamate dehydrogenase 1, | XP_392776 | EKITRRFTLELAKKG | 210 |
| | mitochondrial | | | |
| P10809 | 60 kDa heat shock protein, | XP_392899 | ILEQSWGSPKITKDG | 211 |
| | mitochondrial. | | | |
| P10398 | Serine/threonine-protein | XP_396892 | QTAQGMDYLHAKNII | 212 |
| | kinase A-Raf | | | |
| P10301 | Ras-related protein R-Ras | XP_393035 | DPTIEDSYTKQCVID | 213 |
| P09467 | Fructose-1,6-bisphosphatase | XP_003249076 | DVHRTLKYGGIFLYP | 214 |
| | 1 | | | |
| P09215 | Protein kinase C delta type | NP_001128420 | TFCGTPDYIAPEII | 215 |
| P08559 | Pyruvate dehydrogenase E1 | XP_623502 | MSDPGTSYRTREEIQ | 216 |
| | component subunit alpha, | | | |
| | somatic form, mitochondrial | | | |
| P08559 | Pyruvate dehydrogenase E1 | XP_623502 | NNGYGMGTSVDRASA | 217 |
| | component subunit alpha, | | | |
| | somatic form, mitochondrial | | | |
| P08559 | Pyruvate dehydrogenase E1 | XP_003251259 | LEMVTYRYYGHSMSD | 218 |
| | component subunit alpha, | | | |
| | somatic form, mitochondrial | | | |
| P08249 | Malate dehydrogenase, | XP_392478 | KAKAGTGSATLSMAY | 219 |
| | mitochondrial | | | |
| P08238 | Heat shock protein HSP | C1JYH6 | KENQKHIYYITGESR | 220 |
| | 90-beta | | | |
| P08109 | Heat shock cognate 71 | NP_001153544 | QGNRTTPSYVAFTDT | 221 |
| | kDa protein | | | |
| P08047 | Transcription factor Sp1 | XP_624316 | KVYGKTSHLRAHLR | 222 |
| P07949 | Proto-oncogene tyrosine- | XP_396123 | ESLADHVYTSKSDVW | 223 |
| | protein kinase receptor | | | |
| | Ret | | | |
| P07949 | Proto-oncogene tyrosine- | XP_396123 | DVYEDDAYLKRSKGR | 224 |
| | protein kinase receptor | | | |
| | Ret | | | |
| P07900 | Heat shock protein HSP | C1JYH6 | NKNDRTLTILDSGIG | 225 |
| | 90-alpha | | | |
| P07895 | Superoxide dismutase | AAP93582 | SIFWCNLSPNGG | 226 |
| | [Mn], mitochondrial | | | |
| P06744 | Glucose-6-phosphate | XP_623552 | GPRVHFVSNIDGTHI | 227 |
| | isomerase | | | |
| P06685 | Sodium/potassium- | GeneMark.hmm18129 | QLDEILRYHTEIVFA | 228 |
| | transporting ATPase | | | |
| | subunit alpha-1 | | | |
| P06576 | ATP synthase subunit | XP_624156 | TSKVALVYGQMNEPP | 229 |
| | beta, mitochondrial | | | |
| P06493 | Cyclin-dependent | XP_003249456 | MKKIRLESDDEGIPS | 230 |
| | kinase 1 | | | |
| P06213 | Insulin receptor | GeneMark.hmm14331 | KTVNKDATDRERIEF | 231 |
| P05771 | Protein kinase C | NP_001128420 | QTEFMGFSFLNPEFV | 232 |
| | beta type | | | |
| P05412 | Transcription factor | XP_003251036 | LNMLKLSSPELEKFI | 233 |
| | AP-1 | | | |
| P05129 | Protein kinase C | XP_396874 | GRTTKTFCGTPEY | 234 |
| | gamma type | | | |
| P05023 | Sodium/potassium- | GeneMark.hmm15984 | ICKTRRNSLFRQGM | 235 |
| | transporting ATPase | | | |
| | subunit alpha-1 | | | |
| P04797 | Glyceraldehyde-3- | XP_393605 | IVEGLMTTVHAVTAT | 236 |
| | phosphate dehydrogenase | | | |
| P04626 | Receptor tyrosine- | GeneMark.hmm19490 | GAFGNVYKGVWVPE | 237 |
| | protein kinase erbB-2 | | | |
| P04406 | Glyceraldehyde-3- | XP_393605 | QNIIPAATGAAKAVG | 238 |
| | phosphate dehydrogenase | | | |
| P04075 | Fructose-bisphosphate | XP_623342 | GILAADESTATIGKR | 239 |
| | aldolase A | | | |
| P04049 | RAF proto-oncogene | XP_396892 | IIHRDLKSNNIFLHD | 240 |
| | serine/threonine-protein | | | |
| | kinase | | | |
| P04040 | Catalase. | AAN76688 | NAKDEIVYCKFHYKT | 241 |
| P00558 | Phosphoglycerate kinase | XP_395047 | YFAKALENPERPFLA | 242 |
| | 1 | | | |
| P00519 | Tyrosine-protein kinase | XP_392652 | RLMRDDTYTAHAGAK | 243 |
| | ABL1 | | | |
| P00519 | Tyrosine-protein kinase | XP_392652 | HKLGGGQYGDVYEAV | 244 |
| | ABL1 | | | |
| P00441 | Superoxide dismutase | AAP93581 | DNTNGCTSAGAHFNP | 245 |
| | [Cu—Zn] | | | |
| P00338 | L-lactate dehydrogenase | XP_394662 | IKLKGYTSWAIGLS | 246 |
| | A chain; LDH-A | | | |
| P00338 | L-lactate dehydrogenase | GeneMark.hmm22493 | KKVIGSAYEVIKLKG | 247 |
| | A chain; LDH-A | | | |
| O96017 | Serine/threonine-protein | XP_624334 | MMKTFCGTPMYVAPE | 248 |
| | kinase Chk2 | | | |
| O96013 | Serine/threonine-protein | XP_001122147 | RRKSLVGTPYWMSPE | 7 |
| | kinase PAK 4 | | | |
| O95819 | Mitogen-activated protein | XP_396948 | VSAQLDRTIGRRNTF | 249 |
| | kinase kinase kinase | | | |
| | kinase 4 | | | |
| O95747 | Serine/threonine-protein | XP_396480 | SRQKVRHTFVGTPCW | 250 |
| | kinase OSR1 | | | |
| O95382 | Mitogen-activated protein | XP_003250315 | GLCPSTETFTGTLQY | 251 |
| | kinase kinase kinase 6 | | | |
| O76039 | Cyclin-dependent kinase- | XP_394980 | NYTEYVATRWYR | 252 |
| | like 5 | | | |
| O76031 | ATP-dependent Clp protease | XP_394615 | QNAMIPQYQMLFSMD | 253 |
| | ATP-binding subunit clpX- | | | |
| | like, mitochondrial | | | |
| O75874 | Isocitrate dehydrogenase | XP_623673 | NVTRSDYLETFEFI | 254 |
| | [NADP] cytoplasmic | | | |
| O75792 | Ribonuclease H2 subunit A | XP_396289 | TEYGSGYPNDPETK | 255 |
| O75716 | Serine/threonine-protein | XP_395536 | AAERCSMPYRAPELF | 256 |
| | kinase 16 | | | |
| O75582 | Ribosomal protein S6 kinase | XP_395099 | DKIFRGYSYVAPSIL | 257 |
| | alpha-5 | | | |
| O75533 | Splicing factor 3B subunit | XP_623732 | PARKLTATPTPIAG | 258 |
| | 1 | | | |
| O75469 | Nuclear receptor subfamily | C0SUE0 | GYHYNALTCEGCKGF | 259 |
| | 1 group I member 2 | | | |
| O75460 | Serine/threonine-protein | XP_392044 | KLQLGRVSFSRRSGV | 260 |
| | kinase/endoribonuclease | | | |
| | IRE1 | | | |
| O75251 | NADH dehydrogenase | XP_392437 | IIVAGTLTNKMAPAL | 261 |
| | [ubiquinone] iron-sulfur | | | |
| | protein 7, mitochondrial | | | |
| O61443 | Mitogen-activated protein | XP_395384 | ENEMTGYVATRWYR | 119 |
| | kinase 14B; | | | |
| O60825 | 6-phosphofructo-2-kinase/ | XP_393078 | RYPRGESYEDLVARL | 262 |
| | fructose-2,6-biphosphatase | | | |
| | 2 | | | |
| O60547 | GDP-mannose 4,6 dehydratase | XP_395164 | VKVNPKYFRPTEVD | 263 |
| O60285 | NUAK family SNF1-like kinase | XP_393444 | EQRLLNTFCGSPLY | 264 |
| | 1 | | | |
| O54950 | 5′-AMP-activated protein | XP_003251654 | NLAAEKTYNNLDVSL | 265 |
| | kinase subunit gamma-1 | | | |
| O54949 | Serine/threonine-protein | GeneMark.hmm15332 | DQNKHMTQEVVTQY | 266 |
| | kinase NLK; Nemo-like kinase | | | |
| O54890 | Integrin beta-3; Platelet | XP_001123130 | DTGENPIYKQATSTF | 267 |
| | membrane glycoprotein IIIa | | | |
| O44514 | Mitogen-activated protein | GeneMark.hmm16997 | DPTLTDYVATRWYR | 268 |
| | kinase pmk-3 | | | |
| O43837 | Isocitrate dehydrogenase | XP_624511 | TKDLGGQSSTTEF | 269 |
| | [NAD] subunit beta, | | | |
| | mitochondrial | | | |
| O43464 | Serine protease HTRA2, | XP_624354 | VYKVIVGSPAHLGGL | 270 |
| | mitochondrial | | | |
| O43318 | Mitogen-activated protein | XP_397248 | CDLNTYMTNNKGSAA | 271 |
| | kinase kinase kinase 7 | | | |
| O43318 | Mitogen-activated protein | XP_397248 | YMTNNKGSAAWMAPE | 272 |
| | kinase kinase kinase 7 | | | |
| O35643 | AP-1 complex subunit beta- | XP_003249811 | VEGQDMLYQSLKLTN | 273 |
| | 1 | | | |
| O35099 | Mitogen-activated protein | XP_003250315 | TETFTGTLQYMAPE | 274 |
| | kinase kinase kinase 5 | | | |
| O17732 | Pyruvate carboxylase 1 | GeneMark.hmm9651 | AIQCRVTTEDPAK | 275 |
| O15264 | Mitogen-activated protein | XP_395384 | EMTGYVATRWYR | 276 |
| | kinase 13 | | | |
| O14920 | Inhibitor of nuclear factor | XP_623135 | ELLWKQTYSCSVDYW | 277 |
| | kappa-B kinase subunit beta | | | |
| O14920 | Inhibitor of nuclear factor | XP_624106 | TFIGTLEYLAPEIIQ | 278 |
| | kappa-B kinase subunit beta | | | |
| O14733 | Dual specificity mitogen- | XP_396834 | LVDSKAKTRSAGCAA | 279 |
| | activated protein kinase | | | |
| | kinase 7 | | | |
| O09127 | Ephrin type-A receptor 8; | Q5D184 | MSYGERPYWNWSNQD | 280 |
| | EPH- and ELK-related kinase | | | |
| O08605 | MAP kinase-interacting | XP_395927 | VATPQLLTPVGSADF | 281 |
| | serine/threonine-protein | | | |
| | kinase 1 | | | |
| O00743 | Serine/threonine-protein | XP_624669 | TVWSAPNYCYRCGNV | 282 |
| | phosphatase 6 catalytic | | | |
| | subunit | | | |
| O00571 | ATP-dependent RNA helicase | CBM36382 | GCHLLVATPGRLVDM | 283 |
| | DDX3X; DEAD box protein 3, | | | |
| | X-chromosomal. | | | |
| O00444 | Serine/threonine-protein | XP_623133 | PDEKHLTMCGTPNY | 284 |
| | kinase PLK4 | | | |
| O00311 | Cell division cycle 7- | XP_003250974 | QTAPRAGTPGFRAPE | 285 |
| | related protein kinase | | | |
| O00267 | Transcription elongation | XP_003249083 | TPMHGSQTPMYENGS | 286 |
| | factor SPT5 | | | |
| O00206 | Toll-like receptor 4 | GeneMark.hmm3850 | LYDGYIVYSERDEDF | 287 |
| NP_ | NADH dehydrogenase | XP_003250306 | EPATINYPFEKGPL | 288 |
| 001099792 | [ubiquinone] iron-sulfur | | | |
| | protein 8, mitochondrial | | | |
| | [Rattus norvegicus]. | | | |

TABLE 2
Peptides that are differentially phosphorylated in G4 vs S88 bees
in both the infected and uninfected samples

| | | SEQ | |
|---|---|---|---|
| | | ID | |
| ID | Peptide | NO: | Accession |
| Toll-like receptor 4; hToll; CD antigen = CD284 | LYDGYIVYSERDEDF | 287 | O00206 |
| MAP kinase-interacting serine/threonine-protein | VATPQLLTPVGSADF | 281 | O08605 |
| kinase 1 | | | |
| Mitogen-activated protein kinase kinase kinase 7 | YMTNNKGSAAWMAPE | 272 | O43318 |
| Serine/threonine-protein kinase NLK; Nemo-like | DQNKHMTQEVVTQY | 266 | O54949 |
| kinase | | | |
| Ribonuclease H2 subunit A; | TEYGSGYPNDPETK | 255 | O75792 |
| Tyrosine-protein kinase ABL1 | HKLGGGQYGDVYEAV | 244 | P00519 |
| Catalase. | NAKDEIVYCKFHYKT | 241 | P04040 |
| Pyruvate dehydrogenase E1 component subunit | MSDPGTSYRTREEIQ | 216 | P08559 |
| alpha, somatic form, mitochondrial | | | |
| Protein kinase C delta type | TFCGTPDYIAPEII | 215 | P09215 |
| Ras-related protein R-Ras | DPTIEDSYTKQCVID | 213 | P10301 |
| Serum response factor | DNKLRRYTTFSKRKT | 203 | P11831 |
| Elongation factor 2 | KVMKFSVSPVVRVAV | 201 | P13639 |
| DNA-directed RNA polymerase II subunit RPB1 | SPNYSPTSPTYSPTS | 180 | P24928 |
| Moesin; Membrane-organizing extension spike | GRDKYKTLREIRKG | 176 | P26038 |
| protein. | | | |
| Mitogen-activated protein kinase 1 | LGVLGSPSPEDLECI | 169 | P28482 |
| Pyruvate dehydrogenase E1 component subunit | NGYGMGTSVDRASAS | 165 | P29804 |
| alpha, somatic form, mitochondrial | | | |
| Pyruvate dehydrogenase E1 component subunit | SMSDPGTSYRTREEI | 164 | P29804 |
| alpha, somatic form, mitochondrial | | | |
| Pyruvate dehydrogenase E1 component subunit | TYRYYGHSMSDPGTS | 166 | P29804 |
| alpha, somatic form, mitochondrial | | | |
| Mu-type opioid receptor | MQTVTNMYIVNLAIA | 157 | P33535 |
| TGF-beta receptor type-1 | MTTSGSGSGLPLLVQ | 151 | P36897 |
| Protein kinase C iota type | REGDTTATFCGTPNY | 143 | P41743 |
| MAP kinase-activated protein kinase 2 | DTLQTPCYTPYY | 136 | P49138 |
| Signal transducer and activator of | KDQAFSKYYTP | 128 | P51692 |
| transcription 5B. | | | |
| Ribosomal protein S6 kinase alpha-3 | TCKTPKDSPGVPPSA | 127 | P51812 |
| Serine/threonine-protein kinase PLK1 | HEGERKKTVCGTPNY | 121 | P53350 |
| Ephrin type-B receptor 1; ELK; EPH-like | YVDPHTYEDPNQAV | 117 | P54762 |
| kinase 6 | | | |
| Ephrin type-A receptor 4 | SYVDPHTYEDPNQAV | 116 | P54764 |
| Elongation factor 2 | GETRFTDTRKDEQER | 113 | P55823 |
| Elongation factor 1-alpha 1 | EMHHEALTEALPGDN | 100 | P68104 |
| Histone H3.3 | ATKAARKSAPSTGGV | 93 | P84243 |
| Forkhead box protein O4 | FRPRASSNASS | 91 | P98177 |
| Transcription factor p65 | RPSDGDCSEPVKFTY | 84 | Q04206 |
| Transcription factor p65 | IQLKRPSDGALSEP | 83 | Q04206 |
| Focal adhesion kinase 1 | IVDEEGDYSTPATRD | 82 | Q05397 |
| Nuclear inhibitor of protein phosphatase 1 | EPKKKKYAKEAWPG | 75 | Q12972 |
| Septin-2 | YPLPDCDSDEDEDYK | 63 | Q15019 |
| Eukaryotic translation initiation factor | PNDYSSTPGGTLFS | 43 | Q60876 |
| 4E-binding protein 1 | | | |
| PRKC apoptosis WT1 regulator protein | LREKRRSTGVVHLPS | 39 | Q62627 |
| Regulator of nonsense transcripts 1 | LSQPGLSQAELSQD | 29 | Q92900 |
| Repressor of RNA polymerase III transcription | PHDLQALSPPQTS | 19 | Q9H063 |
| MAF1 homolog | | | |
| Serine/threonine-protein kinase MARK1 | TPGNKLDTFCGSPPY | 15 | Q9P0L2 |
| Serine/threonine-protein kinase SIK1 | LSTWCGSPPYAAPE | 13 | Q9R1U5 |
| Serine/threonine-protein kinase TBK1 | QEDQQFVSLYGTEEY | 11 | Q9UHD2 |
| Serine/threonine-protein kinase PAK mbt | QELPRRKSLVGTPYW | 8 | Q9VXE5 |
| Ribosomal protein S6 kinase alpha-5 | DKIFRGYSYVAPSIL | 257 | O75582 |
| ATP-dependent Clp protease ATP-binding subunit | QNAMIPQYQMLFSMD | 253 | O76031 |
| clpX-like, mitochondrial | | | |
| L-lactate dehydrogenase A chain | KKVIGSAYEVIKLKG | 247 | P00338 |
| RAF proto-oncogene serine/threonine-protein | IIHRDLKSNNIFLHD | 240 | P04049 |
| kinase; | | | |
| Proto-oncogene c-RAF | | | |
| Protein kinase C beta type | QTEFMGFSFLNPEFV | 232 | P05771 |
| Transcription factor Sp1 | KVYGKTSHLRAHLR | 222 | P08047 |
| Pyruvate dehydrogenase E1 component subunit | LEMVTYRYYGHSMSD | 218 | P08559 |
| alpha, somatic form, mitochondrial | | | |
| Glutamate dehydrogenase 1, mitochondrial | EKITRRFTLELAKKG | 210 | P10860 |
| Serum response factor | LRRYTTFSKRKTGIM | 204 | P11831 |
| Pyruvate kinase isozymes M1/M2 | FSHGTHEYHAETIAN | 200 | P14618 |
| Nuclear factor NF-kappa-B p105 subunit | KALRFRYECEGRS | 190 | P19838 |
| GTP cyclohydrolase 1 | VKDIEMFSMCEHHLV | 187 | P22288 |
| Cyclin-dependent kinase 2 | GVPVRTYTHEIVTLW | 184 | P23437 |
| Mitogen-activated protein kinase 3 | FLTEYVATRWYRAPE | 174 | P27361 |
| Nitric oxide synthase, brain | IARAVKFTSKLFGRA | 167 | P29476 |
| C-C chemokine receptor type 7 | ILHLMCISVDRYWAI | 158 | P32248 |
| Serine/threonine-protein kinase mTOR | IKRLHVSASNLQKAW | 142 | P42345 |
| Mitogen-activated protein kinase 8 | TTFMMTPYVVTRYYR | 141 | P45983 |
| Fatty acid synthase | FSRLGVLSPDCRCKS | 135 | P49327 |
| Cyclin-dependent kinase 9 | NGQPNRYTNRVVTLW | 130 | P50750 |
| Serine/threonine-protein kinase PLK1 | LELCRKRSMMELHKR | 122 | P53350 |
| GTP-binding nuclear protein Ran | DRKVKAKSIVFHRKK | 106 | P62826 |
| Mitogen-activated protein kinase kinase | TRMSAAGTYAWMAPE | 95 | P80192 |
| kinase 9 | | | |
| Peptidyl-prolyl cis-trans isomerase FKBP4 | LAKEKKLYANMFDKF | 86 | Q02790 |
| Serine/threonine-protein kinase 38 | DWVFINYTFKRFEGL | 60 | Q15208 |
| Hypoxia-inducible factor 1-alpha | TFLSKHSLSMKFTY | 55 | Q16665 |
| Leucine-rich repeat serine/threonine-protein | SPVIIVGTHYDISYE | 49 | Q5S007 |
| kinase 2 | | | |
| Casein kinase 2, beta polypeptide | ETKMSSSEEVSWIS | 48 | Q5SRQ6 |
| Proto-oncogene tyrosine-protein kinase ROS | FGLARDIYKNDYYRK | 37 | Q78DX7 |
| Mitogen-activated protein kinase 8 | DLDHERMSYLLYQML | 31 | Q91Y86 |
| Prohibitin-2 | ALSQNPGYLKLRKIR | 23 | Q99623 |
| Single-stranded DNA-binding protein 3 | AREKLALYVYEYLLH | 22 | Q9BWW4 |

TABLE 3
Peptides that are differentially phosphorylated in infected G4
(susceptible bees) vs. infected S88 (tolerant) bees

| | | | | | | |
|---|---|---|---|---|---|---|
| | | SEQ | | | | |
| | | ID | | Fold- | | |
| ID | Peptide | NO | Accession | Change | P up | P down |

A. Peptides with increased phosphorylation in G4 compared to S88 bees

| | | | | | | |
|---|---|---|---|---|---|---|
| RAC-gamma serine/ | TYGRTTKTFCGTPEY | 5 | Q9Y243 | 1.558563 | 9.34E−06 | 0.999991 |
| threonine-protein | | | | | | |
| kinase | | | | | | |
| TGF-beta receptor | MTTSGSGSGLPLLVQ | 151 | P36897 | 1.627669 | 0.000114 | 0.999886 |
| type-1; TGFR-1 | | | | | | |
| Serine/threonine- | QEDQQFVSLYGTEEY | 11 | Q9UHD2 | 1.532602 | 0.00027 | 0.99973 |
| protein kinase | | | | | | |
| TBK1 | | | | | | |
| Nuclear inhibitor | EPKKKKYAKEAWPG | 75 | Q12972 | 1.57601 | 0.00035 | 0.99965 |
| of protein | | | | | | |
| phosphatase 1 | | | | | | |
| Pre-rRNA-processing | FPDEVDTPQDILAK | 51 | Q2NL82 | 1.50958 | 0.000386 | 0.999614 |
| protein TSR1 homolog | | | | | | |
| Protein kinase C | REGDTTATFCGTPNY | 143 | P41743 | 1.90052 | 0.000395 | 0.999605 |
| iota type | | | | | | |
| Histone H3.3 | ATKAARKSAPSTGGV | 93 | P84243 | 1.571554 | 0.000828 | 0.999172 |
| Transcription factor | IQLKRPSDGALSEP | 83 | Q04206 | 1.240087 | 0.000836 | 0.999164 |
| p65 | | | | | | |
| Forkhead box protein | FRPRASSNASS | 91 | P98177 | 1.440255 | 0.000959 | 0.999041 |
| O4 | | | | | | |
| MAP kinase-interacting | VATPQLLTPVGSADF | 281 | O08605 | 1.849755 | 0.001174 | 0.998826 |
| serine/threonine- | | | | | | |
| protein kinase 1 | | | | | | |
| Myoblast determination | VDRRKAATLRERRRL | 197 | P15172 | 1.703859 | 0.001584 | 0.998416 |
| protein 1 | | | | | | |
| Heat shock protein 75 | NLGTIARSGSRAFIE | 45 | Q5XHZ0 | 1.581888 | 0.001971 | 0.998029 |
| kDa, mitochondrial | | | | | | |
| Pyruvate dehydrogenase | NGYGMGTSVDRASAS | 165 | P29804 | 1.436189 | 0.002563 | 0.997437 |
| E1 component subunit | | | | | | |
| alpha, somatic form, | | | | | | |
| mitochondrial; | | | | | | |
| PDHE1-A type I | | | | | | |
| Septin-2 | YPLPDCDSDEDEDYK | 63 | Q15019 | 1.455651 | 0.002635 | 0.997365 |
| Elongation factor | EMHHEALTEALPGDN | 100 | P68104 | 1.458424 | 0.003151 | 0.996849 |
| 1-alpha 1 | | | | | | |
| Ribosomal protein S6 | TCKTPKDSPGVPPSA | 127 | P51812 | 1.284851 | 0.003355 | 0.996645 |
| kinase alpha-3 | | | | | | |
| Histone H3.1 | KQTARKSTGGKAPRK | 98 | P68431 | 1.309093 | 0.00366 | 0.99634 |
| Serine/threonine- | RRKSLVGTPYWMSPE | 7 | Q9VXE5 | 1.383154 | 0.004045 | 0.995955 |
| protein kinase | | | | | | |
| PAK mbt | | | | | | |
| Serine/threonine- | NRRALAYSTVGTPDY | 59 | Q15208 | 1.412127 | 0.004711 | 0.995289 |
| protein kinase 38 | | | | | | |
| Protein disulfide- | EEEIDLSDIDLDE | 61 | Q15084 | 1.544308 | 0.00502 | 0.99498 |
| isomerase A6 | | | | | | |
| Serum response | DNKLRRYTTFSKRKT | 203 | P11831 | 1.615038 | 0.005231 | 0.994769 |
| factor; SRF | | | | | | |
| Ras-related protein | KELQRQASPSIVIAL | 109 | P61020 | 1.546367 | 0.005282 | 0.994718 |
| Rab-5B | | | | | | |
| Serine/threonine- | QSNVAVGTPDYISPE | 46 | Q5VT25 | 1.362557 | 0.007082 | 0.992918 |
| protein kinase | | | | | | |
| MRCK alpha | | | | | | |
| Protein kinase C | TFCGTPDYIAPEII | 215 | P09215 | 1.537081 | 0.007606 | 0.992394 |
| delta type; nPKC- | | | | | | |
| delta | | | | | | |
| Eukaryotic trans- | PNDYSSTPGGTLFS | 43 | Q60876 | 1.847337 | 0.007627 | 0.992373 |
| lation initiation | | | | | | |
| factor 4E-binding | | | | | | |
| protein 1 | | | | | | |
| Ephrin type-A | SYVDPHTYEDPNQAV | 116 | P54764 | 1.426248 | 0.007864 | 0.992136 |
| receptor 4 | | | | | | |
| Mu-type opioid | MQTVTNMYIVNLAIA | 157 | P33535 | 1.404441 | 0.009089 | 0.990911 |
| receptor | | | | | | |
| Elongation factor | KVMKFSVSPVVRVAV | 201 | P13639 | 1.624352 | 0.009982 | 0.990018 |
| 2; EF-2 | | | | | | |
| Serine/threonine- | SRQKVRHTFVGTPCW | 250 | O95747 | 1.254249 | 0.010392 | 0.989608 |
| protein kinase | | | | | | |
| OSR1 | | | | | | |
| Regulator of non- | LSQPGLSQAELSQD | 29 | Q92900 | 1.302781 | 0.010402 | 0.989598 |
| sense transcripts 1 | | | | | | |
| Inhibitor of nuclear | EDQQFVSLYGTEEY | 65 | Q14164 | 1.345133 | 0.010662 | 0.989338 |
| factor kappa-B kinase | | | | | | |
| subunit epsilon | | | | | | |
| MAP kinase-activated | DTLQTPCYTPYY | 136 | P49138 | 1.474675 | 0.012204 | 0.987796 |
| protein kinase 2 | | | | | | |
| Serine/threonine- | LSTWCGSPPYAAPE | 13 | Q9R1U5 | 1.24179 | 0.014109 | 0.985891 |
| protein kinase SIK1 | | | | | | |
| Nuclear inhibitor | LGLPETETELDNLTE | 53 | Q28147 | 1.346726 | 0.014256 | 0.985744 |
| of protein | | | | | | |
| phosphatase 1 | | | | | | |
| Mitogen-activated | KSLVGTPYWMSPE | 3 | Q9Y2U5 | 1.270003 | 0.015242 | 0.984758 |
| protein kinase | | | | | | |
| kinase kinase 2 | | | | | | |
| Toll-like receptor | LYDGYIVYSERDEDF | 287 | O00206 | 1.506777 | 0.016028 | 0.983972 |
| 4 | | | | | | |
| DNA-directed RNA | SPNYSPTSPTYSPTS | 180 | P24928 | 1.679355 | 0.016895 | 0.983105 |
| polymerase II | | | | | | |
| subunit RPB1 | | | | | | |
| Pyruvate dehydro- | TYRYYGHSMSDPGTS | 166 | P29804 | 1.420024 | 0.017068 | 0.982932 |
| genase E1 component | | | | | | |
| subunit alpha, | | | | | | |
| somatic form, | | | | | | |
| mitochondrial | | | | | | |
| Mitogen-activated | HILGVLGSPSPEDL | 170 | P28482 | 1.247609 | 0.018431 | 0.981569 |
| protein kinase 1 | | | | | | |
| Ribonuclease H2 | TEYGSGYPNDPETK | 255 | O75792 | 1.414402 | 0.020827 | 0.979173 |
| subunit A | | | | | | |
| Serine/threonine- | HEGERKKTVCGTPNY | 121 | P53350 | 1.557306 | 0.021863 | 0.978137 |
| protein kinase PLK1; | | | | | | |
| Polo-like kinase 1; | | | | | | |
| PLK-1; Serine/ | | | | | | |
| threonine-protein | | | | | | |
| kinase 13; STPK13. | | | | | | |
| Dual specificity | LVDSKAKTRSAGCAA | 279 | O14733 | 1.487511 | 0.022291 | 0.977709 |
| mitogen-activated | | | | | | |
| protein kinase | | | | | | |
| kinase 7 | | | | | | |
| Glyceraldehyde-3- | QNIIPAATGAAKAVG | 238 | P04406 | 1.390466 | 0.022966 | 0.977034 |
| phosphate dehydro- | | | | | | |
| genase; GAPDH | | | | | | |
| Histone deacetylase | FPLRKTASEPNL | 111 | P56524 | 1.359056 | 0.023162 | 0.976838 |
| 4; HD4 | | | | | | |
| Tyrosine-protein | HKLGGGQYGDVYEAV | 244 | P00519 | 1.315249 | 0.024294 | 0.975706 |
| kinase ABL1 | | | | | | |
| Serine/threonine- | EPHVTRRTPDYFL | 101 | P67775 | 1.312622 | 0.027971 | 0.972029 |
| protein phosphatase | | | | | | |
| 2A catalytic subunit | | | | | | |
| alpha isoform | | | | | | |
| TGF-beta receptor | GQVGTRRYMAPEVLE | 149 | P37173 | 1.275295 | 0.028773 | 0.971227 |
| type-2 | | | | | | |
| Repressor of RNA | PHDLQALSPPQTS | 19 | Q9H063 | 1.467034 | 0.028871 | 0.971129 |
| polymerase III | | | | | | |
| transcription MAF1 | | | | | | |
| homolog. | | | | | | |
| Mitogen-activated | YMTNNKGSAAWMAPE | 272 | O43318 | 1.288272 | 0.028889 | 0.971111 |
| protein kinase | | | | | | |
| kinase kinase 7 | | | | | | |
| Elongation factor | GETRFTDTRKDEQER | 113 | P55823 | 1.341531 | 0.029113 | 0.970887 |
| 2; EF-2 | | | | | | |
| Potassium voltage- | YWGVDELYLESCCQ | 64 | Q14721 | 1.250306 | 0.033272 | 0.966728 |
| gated channel | | | | | | |
| subfamily B member | | | | | | |
| 1 | | | | | | |
| Pyruvate dehydro- | SMSDPGTSYRTREEI | 164 | P29804 | 1.339198 | 0.033417 | 0.966583 |
| genase E1 component | | | | | | |
| subunit alpha, | | | | | | |
| somatic form, | | | | | | |
| mitochondrial | | | | | | |
| Ephrin type-B | YVDPHTYEDPNQAV | 117 | P54762 | 1.439314 | 0.035396 | 0.964604 |
| receptor 1 | | | | | | |
| DNA replication | SFGNKHVTPRTLTS | 177 | P25206 | 1.233968 | 0.038286 | 0.961714 |
| licensing factor | | | | | | |
| MCM3 | | | | | | |
| Splicing factor | PARKLTATPTPIAG | 258 | O75533 | 1.110618 | 0.044238 | 0.955762 |
| 3B subunit 1 | | | | | | |
| PRKC apoptosis | LREKRRSTGVVHLPS | 39 | Q62627 | 1.235436 | 0.047923 | 0.952077 |
| WT1 regulator | | | | | | |
| protein | | | | | | |
| Serine/threonine- | IGDFGLATAKTRWSG | 199 | P15056 | 1.243071 | 0.050689 | 0.949311 |
| protein kinase | | | | | | |
| B-raf | | | | | | |
| Cell division | TPNTILATPFRS | 24 | Q99459 | 1.230597 | 0.051313 | 0.948687 |
| cycle 5-like | | | | | | |
| protein | | | | | | |
| G protein-coupled | LDIEQFSTVKGVNLD | 156 | P34947 | 1.24827 | 0.05176 | 0.94824 |
| receptor kinase 5 | | | | | | |
| Transcription | RPSDGDCSEPVKFTY | 84 | Q04206 | 1.749082 | 0.052894 | 0.947106 |
| factor p65 | | | | | | |
| Serine/threonine- | QELPRRKSLVGTPYW | 8 | Q9VXE5 | 1.237277 | 0.053188 | 0.946812 |
| protein kinase | | | | | | |
| PAK mbt | | | | | | |
| Focal adhesion | IVDEEGDYSTPATRD | 82 | Q05397 | 1.361534 | 0.059549 | 0.940451 |
| kinase 1 | | | | | | |
| Serine/threonine- | DQNKHMTQEVVTQY | 266 | O54949 | 1.319317 | 0.060629 | 0.939371 |
| protein kinase NLK | | | | | | |
| Signal transducer | KDQAFSKYYTP | 128 | P51692 | 1.404992 | 0.061197 | 0.938803 |
| and activator of | | | | | | |
| transcription 5B | | | | | | |
| Histone H4 | RGGVKRISGLIYEET | 107 | P62805 | 1.154386 | 0.061562 | 0.938438 |
| Ephrin type-A | ESATEGAYTTRGGKI | 168 | P29320 | 1.237464 | 0.066933 | 0.933067 |
| receptor 3 | | | | | | |
| Pyruvate dehydro- | MSDPGTSYRTREEIQ | 216 | P08559 | 1.212624 | 0.06826 | 0.93174 |
| genase E1 component | | | | | | |
| subunit alpha, | | | | | | |
| somatic form, | | | | | | |
| mitochondrial | | | | | | |
| Catalase | NAKDEIVYCKFHYKT | 241 | P04040 | 1.120314 | 0.071508 | 0.928492 |
| Sodium/potassium- | ICKTRRNSLFRQGM | 235 | P05023 | 1.294971 | 0.073686 | 0.926314 |
| transporting ATPase | | | | | | |
| subunit alpha-1 | | | | | | |
| Ras-related protein | DPTIEDSYTKQCVID | 213 | P10301 | 1.138789 | 0.080355 | 0.919645 |
| R-Ras | | | | | | |
| Moesin | GRDKYKTLREIRKG | 176 | P26038 | 1.25522 | 0.088658 | 0.911342 |
| Serine/threonine- | TPGNKLDTFCGSPPY | 15 | Q9P0L2 | 1.18696 | 0.089654 | 0.910346 |
| protein kinase | | | | | | |
| MARK1 | | | | | | |
| Mitogen-activated | LGVLGSPSPEDLECI | 169 | P28482 | 1.254447 | 0.089659 | 0.910341 |
| protein kinase 1 | | | | | | |

B. Peptides with decreased phosphorylation in G4 compared to S88 bees

| | | | | | | |
|---|---|---|---|---|---|---|
| Peptidyl-prolyl | LAKEKKLYANMFDKF | 86 | Q02790 | −1.77074 | 0.999992 | 8.35E−06 |
| cis-trans isomerase | | | | | | |
| FKBP4 | | | | | | |
| GTP-binding nuclear | DRKVKAKSIVFHRKK | 106 | P62826 | −1.75566 | 0.999968 | 3.24E−05 |
| protein Ran | | | | | | |
| Nuclear factor NF- | KALRFRYECEGRS | 190 | P19838 | −1.75757 | 0.999915 | 8.52E−05 |
| kappa-B p105 subunit | | | | | | |
| Proto-oncogene | FGLARDIYKNDYYRK | 37 | Q78DX7 | −1.8765 | 0.9996 | 0.0004 |
| tyrosine-protein | | | | | | |
| kinase ROS | | | | | | |
| Fatty acid synthase. | FSRLGVLSPDCRCKS | 135 | P49327 | −1.8577 | 0.999487 | 0.000513 |
| Serine/threonine- | LELCRKRSMMELHKR | 122 | P53350 | −1.47492 | 0.999447 | 0.000553 |
| protein kinase PLK1 | | | | | | |
| Hypoxia-inducible | TFLSKHSLSMKFTY | 55 | Q16665 | −1.96103 | 0.999432 | 0.000568 |
| factor 1-alpha | | | | | | |
| Mitogen-activated | EMTGYVATRWYR | 276 | O15264 | −1.65965 | 0.999313 | 0.000687 |
| protein kinase 13 | | | | | | |
| Single-stranded | AREKLALYVYEYLLH | 22 | Q9BWW4 | −1.75918 | 0.999184 | 0.000816 |
| DNA-binding protein | | | | | | |
| 3 | | | | | | |
| Transcription factor | KVYGKTSHLRAHLR | 222 | P08047 | −3.0084 | 0.999178 | 0.000822 |
| Sp1. | | | | | | |
| GTP cyclohydrolase | VKDIEMFSMCEHHLV | 187 | P22288 | −1.43231 | 0.999116 | 0.000884 |
| 1 | | | | | | |
| Serine/threonine- | IKRLHVSASNLQKAW | 142 | P42345 | −1.28092 | 0.998329 | 0.001671 |
| protein kinase mTOR | | | | | | |
| Glutamate dehydro- | EKITRRFTLELAKKG | 210 | P10860 | −1.27676 | 0.997905 | 0.002095 |
| genase 1, | | | | | | |
| mitochondrial | | | | | | |
| Guanine nucleotide- | LCFSPNRYWLCAAFG | 102 | P63244 | −1.30061 | 0.997573 | 0.002427 |
| binding protein | | | | | | |
| subunit beta-2-like | | | | | | |
| 1 | | | | | | |
| Serine protease | VYKVIVGSPAHLGGL | 270 | 043464 | −1.44973 | 0.997269 | 0.002731 |
| HTRA2, mito- | | | | | | |
| chondrial | | | | | | |
| Serine/threonine- | DWVFINYTFKRFEGL | 60 | Q15208 | −1.35101 | 0.99691 | 0.00309 |
| protein kinase 38 | | | | | | |
| Glyceraldehyde-3- | ISWYDNEYGYSCRVI | 196 | P16858 | −1.29486 | 0.996566 | 0.003434 |
| phosphate dehydro- | | | | | | |
| genase | | | | | | |
| Mitogen-activated | DLDHERMSYLLYQML | 31 | Q91Y86 | −1.51918 | 0.996376 | 0.003624 |
| protein kinase 8 | | | | | | |
| Serine/threonine- | IMRLRKKTLQEDEIA | 71 | Q13188 | −1.18885 | 0.996106 | 0.003894 |
| protein kinase 3 | | | | | | |
| Proto-oncogene | DVYEDDAYLKRSKGR | 224 | P07949 | −1.20112 | 0.995865 | 0.004135 |
| tyrosine-protein | | | | | | |
| kinase receptor | | | | | | |
| Ret | | | | | | |
| Ribosomal protein | DKIFRGYSYVAPSIL | 257 | O75582 | −1.63781 | 0.995542 | 0.004458 |
| S6 kinase alpha-5 | | | | | | |
| Toll-like | LYDAFISYSHKD | 16 | Q9NR97 | −1.53656 | 0.993527 | 0.006473 |
| receptor 8 | | | | | | |
| RAF proto-oncogene | IIHRDLKSNNIFLHD | 240 | P04049 | −1.32199 | 0.993384 | 0.006616 |
| serine/threonine- | | | | | | |
| protein kinase | | | | | | |
| Pyruvate dehydro- | LEMVTYRYYGHSMSD | 218 | P08559 | −1.63604 | 0.993318 | 0.006682 |
| genase E1 component | | | | | | |
| subunit alpha, | | | | | | |
| somatic form, | | | | | | |
| mitochondria | | | | | | |
| Serine/threonine- | MMKTFCGTPMYVAPE | 248 | O96017 | −1.35479 | 0.992587 | 0.007413 |
| protein kinase Chk2 | | | | | | |
| Mitogen-activated | RPTENEMTGYVATRW | 118 | P53778 | −1.28982 | 0.992282 | 0.007718 |
| protein kinase 12 | | | | | | |
| Pyruvate kinase | FSHGTHEYHAETIAN | 200 | P14618 | −1.46217 | 0.990837 | 0.009163 |
| isozymes M1/M2 | | | | | | |
| Mitogen-activated | TRMSAAGTYAWMAPE | 95 | P80192 | −1.17585 | 0.990338 | 0.009662 |
| protein kinase | | | | | | |
| kinase kinase 9 | | | | | | |
| Serine/threonine- | KRKSFIGTPYWMAPE | 17 | Q9NQU5 | −1.59626 | 0.989867 | 0.010133 |
| protein kinase | | | | | | |
| PAK 6 | | | | | | |
| Casein kinase 2, | ETKMSSSEEVSWIS | 48 | Q5SRQ6 | −1.5307 | 0.982001 | 0.017999 |
| beta polypeptide. | | | | | | |
| 6-phosphofructo-2- | RYPRGESYEDLVARL | 262 | O60825 | −1.27593 | 0.977947 | 0.022053 |
| kinase/fructose-2,6- | | | | | | |
| biphosphatase 2 | | | | | | |
| Casein kinase I | KISEKKMSTPVEVLC | 139 | P48729 | −1.16597 | 0.977923 | 0.022077 |
| isoform alpha | | | | | | |
| Mitogen-activated | VSAQLDRTIGRRNTF | 249 | O95819 | −1.2468 | 0.974208 | 0.025792 |
| protein kinase | | | | | | |
| kinase kinase | | | | | | |
| kinase 4 | | | | | | |
| Prohibitin-2 | ALSQNPGYLKLRKIR | 23 | Q99623 | −1.28428 | 0.97141 | 0.02859 |
| Mitogen-activated | TTFMMTPYVVTRYYR | 141 | P45983 | −1.27959 | 0.968617 | 0.031383 |
| protein kinase 8; | | | | | | |
| MAP kinase 8 | | | | | | |
| L-lactate dehydro- | KKVIGSAYEVIKLKG | 247 | P00338 | −1.23749 | 0.966838 | 0.033162 |
| genase A chain | | | | | | |
| Mitogen-activated | ENEMTGYVATRWYR | 119 | P53778 | −1.18517 | 0.965566 | 0.034434 |
| protein kinase 12 | | | | | | |
| Nitric oxide | IARAVKFTSKLFGRA | 167 | P29476 | −1.24613 | 0.962443 | 0.037557 |
| synthase, brain | | | | | | |
| Cyclin-dependent | NGQPNRYTNRVVTLW | 130 | P50750 | −1.27579 | 0.961213 | 0.038787 |
| kinase 9 | | | | | | |
| Cyclin-dependent | GVPVRTYTHEIVTLW | 184 | P23437 | −1.25367 | 0.958339 | 0.041661 |
| kinase 2 | | | | | | |
| Nitric oxide | LPSFWIPSKTPEAK | 2 | Q9Y314 | −1.29083 | 0.955943 | 0.044057 |
| synthase-inter- | | | | | | |
| acting protein | | | | | | |
| Mitogen-activated | FLTEYVATRWYRAPE | 174 | P27361 | −1.32843 | 0.955311 | 0.044689 |
| protein kinase 3 | | | | | | |
| ATP-dependent Clp | QNAMIPQYQMLFSMD | 253 | O76031 | −1.48142 | 0.955296 | 0.044704 |
| protease ATP-binding | | | | | | |
| subunit clpX-like, | | | | | | |
| mitochondrial | | | | | | |
| AP-2 complex | AQITSQVTGQIGWRR | 26 | Q96CW1 | −1.13176 | 0.95173 | 0.04827 |
| subunit mu | | | | | | |
| Mps one binder | FGSRSSKTFKPKKNI | 36 | Q7L9L4 | −1.21828 | 0.949603 | 0.050397 |
| kinase activator- | | | | | | |
| like 1A | | | | | | |
| Serine/threonine- | KLQLGRVSFSRRSGV | 260 | O75460 | −1.15348 | 0.948 | 0.052 |
| protein kinase/ | | | | | | |
| endoribonuclease | | | | | | |
| IRE1 | | | | | | |
| Serum response | LRRYTTFSKRKTGIM | 204 | P11831 | −1.20343 | 0.944893 | 0.055107 |
| factor | | | | | | |
| Dual specificity | KICDFGVSGQLIDSM | 88 | Q02750 | −1.2147 | 0.928279 | 0.071721 |
| mitogen-activated | | | | | | |
| protein kinase | | | | | | |
| kinase 1 | | | | | | |
| Leucine-rich re- | SPVIIVGTHYDISYE | 49 | Q5S007 | −1.11446 | 0.928208 | 0.071792 |
| peat serine/ | | | | | | |
| threonine-protein | | | | | | |
| kinase 2 | | | | | | |
| Cyclin-dependent | YDFEMRLTSVVVTQW | 205 | P11802 | −1.0856 | 0.926316 | 0.073684 |
| kinase 4 | | | | | | |
| Nuclear factor | YIQLKRPSDGATSEP | 27 | Q94527 | −1.25446 | 0.92616 | 0.07384 |
| NF-kappa-B p110 | | | | | | |
| subunit | | | | | | |
| Protein kinase | QTEFMGFSFLNPEFV | 232 | P05771 | −1.2312 | 0.918594 | 0.081406 |
| C beta type | | | | | | |
| Serine/threonine- | IRSRPPYTDYVSTRW | 10 | Q9UPZ9 | −1.16859 | 0.915155 | 0.084845 |
| protein kinase ICK | | | | | | |
| Beta-adrenergic | AVLADVSYLMAMEKS | 178 | P25098 | −1.24674 | 0.913952 | 0.086048 |
| receptor kinase 1 | | | | | | |
| 78 kDa glucose- | VFDLGGGTFDVSLLT | 209 | P11021 | −1.19556 | 0.913251 | 0.086749 |
| regulated protein | | | | | | |
| Succinate dehydro- | YKERIDEYDYAKPLE | 30 | Q920L2 | −1.21906 | 0.909049 | 0.090951 |
| genase [ubiquinone] | | | | | | |
| flavoprotein sub- | | | | | | |
| unit, mitochondrial | | | | | | |
| Sodium-dependent | SLWKGISTSGKVVW | 161 | P31645 | −1.15792 | 0.908302 | 0.091698 |
| serotonin transporter | | | | | | |
| C-C chemokine | ILHLMCISVDRYWAI | 158 | P32248 | −1.21711 | 0.90348 | 0.09652 |
| receptor type 7 | | | | | | |

TABLE 4
Peptides that are differentially phosphorylated in uninfected G4
(susceptible) vs uninfected S88 (tolerant) bees

| | | SEQ | | | | |
|---|---|---|---|---|---|---|
| | | ID | | Fold- | | |
| ID | Peptide | NO | Accession | Change | P up | P down |

A. Peptides with increased phosphorylation in G4 compared to S88 bees

| | | | | | | |
|---|---|---|---|---|---|---|
| Serine/threonine- | LGQQDRSSSAPNV | 172 | P28028 | 1.766066 | 3.77E−06 | 0.999996 |
| protein kinase B-raf | | | | | | |
| E3 ubiquitin-protein | IDHNTRTTQWEDPR | 34 | Q8C863 | 1.441036 | 4.34E−06 | 0.999996 |
| ligase Itchy | | | | | | |
| Ras-related protein | DPTIEDSYTKQCVID | 213 | P10301 | 1.8221 | 3.65E−05 | 0.999963 |
| R-Ras; p23 | | | | | | |
| Glucose-6-phosphate | DLTYGSRYKDLKLPD | 207 | P11413 | 1.670976 | 5.30E−05 | 0.999947 |
| 1-dehydrogenase | | | | | | |
| DNA repair protein | ETRICKIYDSPCLPE | 78 | Q06609 | 1.956526 | 5.52E−05 | 0.999945 |
| RAD51 homolog 1 | | | | | | |
| Serine/threonine- | DQNKHMTQEVVTQY | 266 | O54949 | 2.009532 | 9.86E−05 | 0.999901 |
| protein kinase NLK | | | | | | |
| Mitogen-activated | YMTNNKGSAAWMAPE | 272 | O43318 | 2.067461 | 0.000104 | 0.999896 |
| protein kinase | | | | | | |
| kinase kinase 7 | | | | | | |
| GDP-mannose 4,6 | VKVNPKYFRPTEVD | 263 | O60547 | 1.605958 | 0.000113 | 0.999887 |
| dehydratase | | | | | | |
| Programmed cell | KKDNDFIYHERIPDI | 32 | Q8WUM4 | 1.477928 | 0.000167 | 0.999833 |
| death 6-inter- | | | | | | |
| acting protein | | | | | | |
| Ribonuclease H2 | TEYGSGYPNDPETK | 255 | O75792 | 1.621166 | 0.000211 | 0.999789 |
| subunit A | | | | | | |
| AP-2 complex | AQITSQVTGQIGWRR | 26 | Q96CW1 | 1.379532 | 0.000284 | 0.999716 |
| subunit mu | | | | | | |
| Signal transducer | KDQAFSKYYTP | 128 | P51692 | 2.680111 | 0.000378 | 0.999622 |
| and activator of | | | | | | |
| transcription 5B. | | | | | | |
| DNA-directed | SPNYSPTSPTYSPTS | 180 | P24928 | 2.471939 | 0.000393 | 0.999607 |
| RNA polymerase | | | | | | |
| II subunit RPB1 | | | | | | |
| Mitogen-activated | HAGFLTEYVATRWYR | 72 | Q13164 | 1.249692 | 0.000454 | 0.999546 |
| protein kinase 7 | | | | | | |
| Pyruvate dehydro- | MSDPGTSYRTREEIQ | 216 | P08559 | 1.56794 | 0.00055 | 0.99945 |
| genase E1 component | | | | | | |
| subunit alpha, | | | | | | |
| somatic form, | | | | | | |
| mitochondrial | | | | | | |
| Pyruvate dehydro- | SMSDPGTSYRTREEI | 164 | P29804 | 1.599686 | 0.000675 | 0.999325 |
| genase E1 component | | | | | | |
| subunit alpha, | | | | | | |
| somatic form, | | | | | | |
| mitochondrial | | | | | | |
| Serine/threonine- | PFQGDNIYKLYENIG | 6 | Q9WTK7 | 1.279065 | 0.000727 | 0.999273 |
| protein kinase 11 | | | | | | |
| Focal adhesion | IVDEEGDYSTPATRD | 82 | Q05397 | 1.813771 | 0.000805 | 0.999195 |
| kinase 1 | | | | | | |
| Glycogen phos- | QEKRKQISVRGIVDV | 208 | P11217 | 2.214951 | 0.000831 | 0.999169 |
| phorylase, | | | | | | |
| muscle form | | | | | | |
| Dual specificity | VSGQLIDSMANSFVG | 87 | Q02750 | 1.340064 | 0.000873 | 0.999127 |
| mitogen-activated | | | | | | |
| protein kinase | | | | | | |
| kinase 1 | | | | | | |
| Ephrin type-B | YVDPHTYEDPNQAV | 117 | P54762 | 1.986284 | 0.000885 | 0.999115 |
| receptor 1 | | | | | | |
| Pyruvate dehydro- | SMSDPGTSYRTREEV | 175 | P26267 | 1.980084 | 0.000915 | 0.999085 |
| genase E1 component | | | | | | |
| subunit alpha | | | | | | |
| type I, | | | | | | |
| mitochondrial | | | | | | |
| MAP kinase-activated | DTLQTPCYTPYY | 136 | P49138 | 1.851384 | 0.000938 | 0.999062 |
| protein kinase 2 | | | | | | |
| Pyruvate dehydro- | TYRYYGHSMSDPGTS | 166 | P29804 | 1.60907 | 0.001042 | 0.998958 |
| genase E1 component | | | | | | |
| subunit alpha, | | | | | | |
| somatic form, | | | | | | |
| mitochondrial | | | | | | |
| Ribosomal protein | TCKTPKDSPGVPPSA | | P51812 | 1.559271 | 0.001052 | 0.998948 |
| S6 kinase alpha-3 | | | | | | |
| Sodium/potassium- | QLDEILRYHTEIVFA | 228 | P06685 | 1.486436 | 0.001101 | 0.998899 |
| transporting ATPase | | | | | | |
| subunit alpha-1 | | | | | | |
| Eukaryotic | PNDYSSTPGGTLFS | 43 | Q60876 | 2.35319 | 0.001113 | 0.998887 |
| translation | | | | | | |
| initiation factor | | | | | | |
| 4E-binding protein 1 | | | | | | |
| Regulator of non- | LSQPGLSQAELSQD | 29 | Q92900 | 2.108874 | 0.001166 | 0.998834 |
| sense transcripts 1 | | | | | | |
| Pyruvate dehydro- | NGYGMGTSVDRASAS | 165 | P29804 | 1.437924 | 0.001171 | 0.998829 |
| genase E1 component | | | | | | |
| subunit alpha, | | | | | | |
| somatic form, | | | | | | |
| mitochondrial | | | | | | |
| Pyruvate dehydro- | NNGYGMGTSVDRASA | 217 | P08559 | 1.57173 | 0.001283 | 0.998717 |
| genase E1 component | | | | | | |
| subunit alpha, | | | | | | |
| somatic form, | | | | | | |
| mitochondrial | | | | | | |
| DNA excision repair | GANRWIYDPDWNPA | 85 | Q03468 | 1.470528 | 0.001386 | 0.998614 |
| protein ERCC-6 | | | | | | |
| Elongation factor | KVMKFSVSPWRVAV | 201 | P13639 | 1.386269 | 0.001433 | 0.998567 |
| 2 | | | | | | |
| Ribosomal protein | DSEFTCKTPKDSPGV | 126 | P51812 | 1.692344 | 0.001535 | 0.998465 |
| S6 kinase alpha-3 | | | | | | |
| Nuclear inhibitor | EPKKKKYAKEAWPG | 75 | Q12972 | 2.144991 | 0.001592 | 0.998408 |
| of protein phos- | | | | | | |
| phatase 1 | | | | | | |
| Transcription | RPSDGDCSEPVKFTY | 84 | Q04206 | 2.891473 | 0.001689 | 0.998311 |
| factor p65 | | | | | | |
| Fructose-1,6- | DVHRTLKYGGIFLYP | 214 | P09467 | 1.586253 | 0.001779 | 0.998221 |
| bisphosphatase 1. | | | | | | |
| Serine/threonine- | HEGERKKTVCGTPNY | 121 | P53350 | 2.21433 | 0.002368 | 0.997632 |
| protein kinase PLK1 | | | | | | |
| Transcription | IQLKRPSDGALSEP | 83 | Q04206 | 1.297039 | 0.002383 | 0.997617 |
| factor p65 | | | | | | |
| Cyclin-dependent | EKIGEGTYGVVYKAK | 179 | P24941 | 1.528721 | 0.002388 | 0.997612 |
| kinase 2 | | | | | | |
| Septin-2 | YPLPDCDSDEDEDYK | 63 | Q15019 | 1.620478 | 0.003103 | 0.996897 |
| Mps one binder | FGSRSSKTFKPKKNI | 36 | Q7L9L4 | 1.405526 | 0.003155 | 0.996845 |
| kinase activator- | | | | | | |
| like 1A | | | | | | |
| Repressor of RNA | PHDLQALSPPQTS | 19 | Q9H063 | 1.470902 | 0.003971 | 0.996029 |
| polymerase III | | | | | | |
| transcription MAF1 | | | | | | |
| homolog | | | | | | |
| Ephrin type-A | SYVDPHTYEDPNQAV | 116 | P54764 | 1.736316 | 0.004007 | 0.995993 |
| receptor 4 | | | | | | |
| Protein kinase C | REGDTTATFCGTPNY | 143 | P41743 | 1.345067 | 0.004024 | 0.995976 |
| iota type | | | | | | |
| Protein phospha- | KPLLRRKSDLPQDTY | 35 | Q7TNL5 | 1.262237 | 0.004108 | 0.995892 |
| tase 2A B56 delta | | | | | | |
| subunit | | | | | | |
| Nuclear receptor | GYHYNALTCEGCKGF | 259 | O75469 | 1.630731 | 0.004796 | 0.995204 |
| subfamily 1 group | | | | | | |
| I member 2 | | | | | | |
| Peptidyl-prolyl | GWEKRLSRSTGQHY | 69 | Q13526 | 1.416813 | 0.005594 | 0.994406 |
| cis-trans | | | | | | |
| isomerase NIMA- | | | | | | |
| interacting 1 | | | | | | |
| Phosphatidylinositol | ENLDLKLTPYRVLAT | 33 | Q8NEB9 | 1.510151 | 0.006148 | 0.993852 |
| 3-kinase catalytic | | | | | | |
| subunit type 3 | | | | | | |
| Proto-oncogene | DVYEDDAYLKRSKGR | 224 | P07949 | 1.242623 | 0.006732 | 0.993268 |
| tyrosine-protein | | | | | | |
| kinase receptor Ret | | | | | | |
| Tyrosine-protein | HKLGGGQYGDVYEAV | 244 | P00519 | 1.363994 | 0.007102 | 0.992898 |
| kinase ABL1 | | | | | | |
| Forkhead box | FRPRASSNASS | 91 | P98177 | 1.26224 | 0.008915 | 0.991085 |
| protein O4. | | | | | | |
| Serine/threonine- | QEDQQFVSLYGTEEY | 11 | Q9UHD2 | 1.442819 | 0.008992 | 0.991008 |
| protein kinase TBK1 | | | | | | |
| RAC-gamma serine/ | TYGRTTKTFCGTPEY | 5 | Q9Y243 | 1.483802 | 0.010667 | 0.989333 |
| threonine-protein | | | | | | |
| kinase | | | | | | |
| Histone H3.3. | ATKAARKSAPSTGGV | 93 | P84243 | 1.332905 | 0.010942 | 0.989058 |
| TGF-beta receptor | MTTSGSGSGLPLLVQ | 151 | P36897 | 1.426145 | 0.012165 | 0.987835 |
| type-1 | | | | | | |
| Heat shock protein | KENQKHIYYITGESR | 220 | P08238 | 1.254418 | 0.012177 | 0.987823 |
| HSP 90-beta | | | | | | |
| Heat shock cognate | QGNRTTPSYVAFTDT | 221 | P08109 | 1.26466 | 0.01696 | 0.98304 |
| 71 kDa protein. | | | | | | |
| Serine/threonine- | KLQLGRVSFSRRSGV | 260 | O75460 | 1.201041 | 0.018427 | 0.981573 |
| protein kinase/ | | | | | | |
| endoribonuclease | | | | | | |
| IRE1 | | | | | | |
| Inhibitor of nuclear | ELLWKQTYSCSVDYW | 277 | O14920 | 1.224599 | 0.018819 | 0.981181 |
| factor kappa-B | | | | | | |
| kinase subunit beta | | | | | | |
| Catalase. | NAKDEIVYCKFHYKT | 241 | P04040 | 1.179741 | 0.019623 | 0.980377 |
| Salt-inducible | LSTWCGSPPYAAPE | 13 | Q3LRT3 | 1.296258 | 0.019839 | 0.980161 |
| kinase 2. | | | | | | |
| Heat shock protein | NKNDRTLTILDSGIG | 225 | P07900 | 1.296646 | 0.02228 | 0.97772 |
| HSP 90-alpha | | | | | | |
| Cytoplasmic tyrosine- | RYVLDDQYTSSGGTK | 80 | P51813 | 1.246303 | 0.023883 | 0.976117 |
| protein kinase BMX | | | | | | |
| Proto-oncogene | ESLADHVYTSKSDVW | 223 | P07949 | 1.434404 | 0.024112 | 0.975888 |
| tyrosine-protein | | | | | | |
| kinase receptor Ret | | | | | | |
| SNW domain-con- | KIPRGPPSPPAPVMH | 67 | Q13573 | 1.363721 | 0.02482 | 0.97518 |
| taining protein | | | | | | |
| 1 | | | | | | |
| Isocitrate de- | NVTRSDYLETFEFI | 254 | O75874 | 1.198717 | 0.025187 | 0.974813 |
| hydrogenase [NADP] | | | | | | |
| cytoplasmic | | | | | | |
| Integrin beta-3 | DTGENPIYKQATSTF | 267 | O54890 | 1.277057 | 0.025799 | 0.974201 |
| MAP kinase-inter- | VATPQLLTPVGSADF | 281 | O08605 | 1.190134 | 0.027297 | 0.972703 |
| acting serine/ | | | | | | |
| threonine-protein | | | | | | |
| kinase 1 | | | | | | |
| Guanine nucleotide- | RRREYQLTDSAKYYL | 163 | P29992 | 1.185984 | 0.028649 | 0.971351 |
| binding protein | | | | | | |
| subunit alpha-11 | | | | | | |
| Clathrin heavy | LLIDEEDYQGLRTSI | 89 | Q00610 | 1.150992 | 0.030052 | 0.969948 |
| chain 1 | | | | | | |
| NADH dehydrogenase | IIVAGTLTNKMAPAL | 261 | O75251 | 1.263622 | 0.030293 | 0.969707 |
| [ubiquinone] iron- | | | | | | |
| sulfur protein 7, | | | | | | |
| mitochondrial | | | | | | |
| Mitogen-activated | APEIMLNSKGYTKSI | 173 | P27361 | 1.405383 | 0.031343 | 0.968657 |
| protein kinase 3 | | | | | | |
| Serine/threonine- | TPGNKLDTFCGSPPY | 15 | Q9POL2 | 1.374169 | 0.032463 | 0.967537 |
| protein kinase | | | | | | |
| MARK1. | | | | | | |
| Mitogen-activated | LGVLGSPSPEDLECI | 169 | P28482 | 1.624047 | 0.032903 | 0.967097 |
| protein kinase 1 | | | | | | |
| Serum response | DNKLRRYTTFSKRKT | 203 | P11831 | 1.306421 | 0.036316 | 0.963684 |
| factor | | | | | | |
| Cyclin-dependent | FGIPVRVYTHEVVTL | 181 | P23572 | 1.194021 | 0.038716 | 0.961284 |
| kinase 1 | | | | | | |
| Superoxide dis- | SIFWCNLSPNGG | 226 | P07895 | 1.259974 | 0.040307 | 0.959693 |
| mutase [Mn], | | | | | | |
| mitochondrial | | | | | | |
| Myoblast deter- | VDRRKAATLRERRRL | 197 | P15172 | 1.271266 | 0.040667 | 0.959333 |
| mination protein | | | | | | |
| 1 | | | | | | |
| Nuclear factor | YIQLKRPSDGATSEP | 27 | Q94527 | 1.28773 | 0.041986 | 0.958014 |
| NF-kappa-B p110 | | | | | | |
| subunit | | | | | | |
| Serine/threonine- | APSSRRNTLCGTLDY | 110 | P59241 | 1.250586 | 0.042922 | 0.957078 |
| protein kinase 6 | | | | | | |
| E3 ubiquitin- | TAEQYELYCEMGSTF | 186 | P22681 | 1.219182 | 0.044197 | 0.955803 |
| protein ligase | | | | | | |
| CBL | | | | | | |
| Protein kinase C | TFCGTPDYIAPEII | 215 | P09215 | 1.243487 | 0.051722 | 0.948278 |
| delta type | | | | | | |
| Serine/threonine- | LSTWCGSPPYAAPE | 13 | Q9R1U5 | 1.25652 | 0.053036 | 0.946964 |
| protein kinase SIK1 | | | | | | |
| Mu-type opioid | MQTVTNMYIVNLAIA | 157 | P33535 | 1.154793 | 0.053291 | 0.946709 |
| receptor; | | | | | | |
| Ribosomal protein | NRVFQGFTYVAPSIL | 182 | P23443 | 1.274365 | 0.054173 | 0.945827 |
| S6 kinase beta-1 | | | | | | |
| Toll-like | LYDGYIVYSERDEDF | 287 | O00206 | 1.518828 | 0.054574 | 0.945426 |
| receptor 4 | | | | | | |
| Inhibitor of | TFIGTLEYLAPEIIQ | 278 | O14920 | 1.467802 | 0.054632 | 0.945368 |
| nuclear factor | | | | | | |
| kappa-B kinase | | | | | | |
| subunit beta | | | | | | |
| Tyrosine-protein | GIANIAISPTIIRKN | 41 | Q62120 | 1.126907 | 0.055106 | 0.944894 |
| kinase JAK2 | | | | | | |
| Moesin | GRDKYKTLREIRKG | 176 | P26038 | 1.22714 | 0.056868 | 0.943132 |
| 60 kDa heat | ILEQSWGSPKITKDG | 211 | P10809 | 1.127824 | 0.05855 | 0.94145 |
| shock protein, | | | | | | |
| mitochondrial | | | | | | |
| PRKC apoptosis | LREKRRSTGVVHLPS | 39 | Q62627 | 1.255019 | 0.061868 | 0.938132 |
| WT1 regulator | | | | | | |
| protein | | | | | | |
| Elongation factor | EMHHEALTEALPGDN | 100 | P68104 | 1.163109 | 0.062264 | 0.937736 |
| 1-alpha 1 | | | | | | |
| Mitogen-activated | GSLVGTLNYVAPE | 123 | P53349 | 1.328786 | 0.06234 | 0.93766 |
| protein kinase | | | | | | |
| kinase kinase 1 | | | | | | |
| Stress-70 protein, | VIGIDLGTTFSCVAV | 148 | P38646 | 1.18998 | 0.063816 | 0.936184 |
| mitochondrial | | | | | | |
| Mitogen-activated | CDLNTYMTNNKGSAA | 271 | O43318 | 1.325024 | 0.069912 | 0.930088 |
| protein kinase | | | | | | |
| kinase kinase 7 | | | | | | |
| Transitional endo- | AMRFARRSVSDNDIR | 115 | P55072 | 1.204629 | 0.073837 | 0.926163 |
| plasmic reticulum | | | | | | |
| ATPase | | | | | | |
| Peptidyl-prolyl | SHLLVKHSGSRRPSS | 70 | Q13526 | 1.093763 | 0.074759 | 0.925241 |
| cis-trans isomerase | | | | | | |
| NIMA-interacting 1 | | | | | | |
| Elongation factor | GETRFTDTRKDEQER | 113 | P55823 | 1.179299 | 0.076496 | 0.923504 |
| 2 | | | | | | |
| Succinate dehydro- | YKERIDEYDYAKPLE | 30 | Q920L2 | 1.166456 | 0.080982 | 0.919018 |
| genase [ubiquinone] | | | | | | |
| flavoprotein sub- | | | | | | |
| unit, mitochondrial | | | | | | |
| Mitogen-activated | ATINKRKSFIGTPYW | 28 | Q92918 | 1.267777 | 0.084046 | 0.915954 |
| protein kinase | | | | | | |
| kinase kinase kinase | | | | | | |
| 1 | | | | | | |
| Serine/threonine- | QELPRRKSLVGTPYW | 8 | Q9VXE5 | 1.254387 | 0.089305 | 0.910695 |
| protein kinase | | | | | | |
| PAK mbt | | | | | | |
| Cryptochrome-1 | SLRKLNSRLFVIRG | 92 | P97784 | 1.084799 | 0.089991 | 0.910009 |
| Peroxiredoxin-1 | HLAWVNTPRKQGGL | 77 | Q06830 | 1.166858 | 0.09347 | 0.90653 |

B. Peptides with decreased phosphorylation in G4 compared to S88 bees

| | | | | | | |
|---|---|---|---|---|---|---|
| GTP-binding nuclear | DRKVKAKSIVFHRKK | 106 | P62826 | −1.55749 | 0.999802 | 0.000198 |
| protein Ran | | | | | | |
| Myosin-VI | GGIKGTVIMVPLK | 52 | Q29122 | −1.72454 | 0.999695 | 0.000305 |
| Mitogen-activated | DLDHERMSYLLYQML | 31 | Q91Y86 | −2.02534 | 0.999594 | 0.000406 |
| protein kinase 8 | | | | | | |
| Pyruvate dehydro- | LEMVTYRYYGHSMSD | 218 | P08559 | −2.05804 | 0.99956 | 0.00044 |
| genase E1 component | | | | | | |
| subunit alpha, | | | | | | |
| somatic form, | | | | | | |
| mitochondrial | | | | | | |
| Transcription factor | LNMLKLSSPELEKFI | 233 | P05412 | −1.6871 | 0.999412 | 0.000588 |
| AP-1 | | | | | | |
| GTP cyclohydrolase 1 | VKDIEMFSMCEHHLV | 187 | P22288 | −1.93832 | 0.999355 | 0.000645 |
| Hypoxia-inducible | TFLSKHSLSMKFTY | 55 | Q16665 | −1.77723 | 0.999327 | 0.000673 |
| factor 1-alpha | | | | | | |
| Fructose-bisphosphate | GILAADESTATIGKR | 239 | P04075 | −1.26823 | 0.99911 | 0.00089 |
| aldolase A | | | | | | |
| Cell division cycle | PLKGGLNTPLNNSDF | 25 | Q99459 | −1.49739 | 0.999079 | 0.000921 |
| 5-like protein | | | | | | |
| Toll-like receptor | LYDAFISYSHKD | 16 | Q9NR97 | −2.23575 | 0.99891 | 0.00109 |
| 8 | | | | | | |
| Single-stranded | AREKLALYVYEYLLH | 22 | Q9BWW4 | −2.04669 | 0.998857 | 0.001143 |
| DNA-binding protein | | | | | | |
| 3 | | | | | | |
| Pyruvate kinase | FSHGTHEYHAETIAN | 200 | P14618 | −1.52817 | 0.998803 | 0.001197 |
| isozymes M1/M2 | | | | | | |
| Caspase-9 | LRSRCGTNEDCKNL | 114 | P55211 | −1.35466 | 0.998764 | 0.001236 |
| 2-oxoisovalerate | TYRIGHHSTSDDST | 202 | P11960 | −1.59252 | 0.998647 | 0.001353 |
| dehydrogenase | | | | | | |
| subunit alpha, | | | | | | |
| mitochondrial | | | | | | |
| 6-phosphofructo-2- | RYPRGESYEDLVARL | 262 | O60825 | −1.42496 | 0.998637 | 0.001363 |
| kinase/fructose-2,6- | | | | | | |
| biphosphatase 2 | | | | | | |
| Ribosomal protein S6 | DKIFRGYSYVAPSIL | 257 | O75582 | −1.35529 | 0.998636 | 0.001364 |
| kinase alpha-5 | | | | | | |
| Serum response | LRRYTTFSKRKTGIM | 204 | P11831 | −1.56292 | 0.998397 | 0.001603 |
| factor | | | | | | |
| Serine/threonine- | MMKTFCGTPMYVAPE | 248 | O96017 | −1.73747 | 0.998377 | 0.001623 |
| protein kinase Chk2 | | | | | | |
| Cyclin-dependent | GVPVRTYTHEIVTLW | 184 | P23437 | −1.16648 | 0.998368 | 0.001632 |
| kinase 2 | | | | | | |
| Eukaryotic initiation | GQHWSGTPGRVFDM | 147 | P38919 | −1.43178 | 0.998354 | 0.001646 |
| factor 4A-III | | | | | | |
| Glyceraldehyde-3- | IVEGLMTTVHAVTAT | 236 | P04797 | −1.4258 | 0.99824 | 0.00176 |
| phosphate dehydro- | | | | | | |
| genase; GAPDH. | | | | | | |
| Peptidyl-prolyl | LAKEKKLYANMFDKF | 86 | Q02790 | −1.77047 | 0.998211 | 0.001789 |
| cis-trans isomerase | | | | | | |
| FKBP4 | | | | | | |
| cGMP-dependent | GRKTWTFCGTPEY | 66 | Q13976 | −1.27886 | 0.997407 | 0.002593 |
| protein kinase 1 | | | | | | |
| Cyclin-dependent | NGQPNRYTNRVVTLW | 130 | P50750 | −1.413 | 0.997363 | 0.002637 |
| kinase 9 | | | | | | |
| Fatty acid synthase | FSRLGVLSPDCRCKS | 135 | P49327 | −1.69401 | 0.997069 | 0.002931 |
| Proto-oncogene | FGLARDIYKNDYYRK | 37 | Q78DX7 | −2.13873 | 0.997047 | 0.002953 |
| tyrosine-protein | | | | | | |
| kinase ROS | | | | | | |
| Chromobox protein | GYSNEENTVVEPEENL | 94 | P83916 | −1.33523 | 0.996899 | 0.003101 |
| homolog 1 | | | | | | |
| RAF proto-oncogene | IIHRDLKSNNIFLHD | 240 | P04049 | −1.38407 | 0.99674 | 0.00326 |
| serine/threonine- | | | | | | |
| protein kinase | | | | | | |
| RAC-alpha serine/ | HFPQFSYQESHSA | 159 | P31749 | −1.60686 | 0.996294 | 0.003706 |
| threonine-protein | | | | | | |
| kinase | | | | | | |
| Serine/threonine- | TPGNKLDTFCGSPPY | 15 | Q9P0L2 | −1.49591 | 0.995422 | 0.004578 |
| protein kinase | | | | | | |
| MARK1 | | | | | | |
| Serine/threonine- | LLLALDGTLKISDFG | 58 | Q15831 | −1.29365 | 0.995227 | 0.004773 |
| protein kinase 11 | | | | | | |
| Transcription | KVYGKTSHLRAHLR | 222 | P08047 | −2.2351 | 0.99506 | 0.00494 |
| factor Sp1 | | | | | | |
| Serine/threonine- | LELCRKRSMMELHKR | 122 | P53350 | −1.9477 | 0.994885 | 0.005115 |
| protein kinase | | | | | | |
| PLK1 | | | | | | |
| Serine/threonine- | HRDIKSDSILLTADG | 14 | Q9P286 | −1.39822 | 0.994468 | 0.005532 |
| protein kinase | | | | | | |
| PAK 7 | | | | | | |
| 60S ribosomal | KIGPLGLSPKKVGDD | 162 | P30050 | −1.2895 | 0.994418 | 0.005582 |
| protein L12 | | | | | | |
| Rho GDP-dissocia- | GKVARGSYSVSSLF | 124 | P52565 | −1.50111 | 0.994348 | 0.005652 |
| tion inhibitor 1 | | | | | | |
| Tyrosine-protein | GSLLTYLRKNTNT | 40 | Q62120 | −1.33912 | 0.993892 | 0.006108 |
| kinase JAK2 | | | | | | |
| Mitogen-activated | LAREVYKTTRMSAAG | 56 | Q16584 | −1.35904 | 0.993636 | 0.006364 |
| protein kinase | | | | | | |
| kinase kinase 11 | | | | | | |
| L-lactate dehydro- | KKVIGSAYEVIKLKG | 247 | P00338 | −1.70655 | 0.993035 | 0.006965 |
| genase A chain | | | | | | |
| Receptor tyrosine- | GAFGNVYKGVWVPE | 237 | P04626 | −1.30834 | 0.992938 | 0.007062 |
| protein kinase | | | | | | |
| erbB-2 | | | | | | |
| Prohibitin-2 | ALSQNPGYLKLRKIR | 23 | Q99623 | −1.42565 | 0.992743 | 0.007257 |
| Mitogen-activated | TENEMTGYVATRWYR | 54 | Q17446 | −1.54305 | 0.992648 | 0.007352 |
| protein kinase | | | | | | |
| pmk-1 | | | | | | |
| Vesicle-fusing | MNRLIKASSKVEVD | 140 | P46460 | −1.43051 | 0.992464 | 0.007536 |
| ATPase | | | | | | |
| T-complex protein | GSRVRVDSMAKIAEL | 96 | P78371 | −1.41942 | 0.991505 | 0.008495 |
| 1 subunit beta | | | | | | |
| Ubiquitin-conju- | LDEPNPNSPANSLAA | 134 | P49459 | −1.47227 | 0.99094 | 0.00906 |
| gating enzyme E2 A | | | | | | |
| Ras-related C3 | YDRLRPLSYPQTDVF | 104 | P63000 | −1.28285 | 0.989971 | 0.010029 |
| botulinum toxin | | | | | | |
| substrate 1 | | | | | | |
| Mitogen-activated | KSLVGTPYWMSPE | 3 | Q9Y2U5 | −1.29807 | 0.989245 | 0.010755 |
| protein kinase | | | | | | |
| kinase kinase 2 | | | | | | |
| Leucine-rich repeat | SPVIIVGTHYDISYE | 49 | Q5S007 | −1.25873 | 0.989241 | 0.010759 |
| serine/threonine- | | | | | | |
| protein kinase 2 | | | | | | |
| Serine/threonine- | IKRLHVSASNLQKAW | 142 | P42345 | −1.58382 | 0.988507 | 0.011493 |
| protein kinase mTOR | | | | | | |
| Phosphoglycerate | YFAKALENPERPFLA | 242 | P00558 | −1.5591 | 0.988473 | 0.011527 |
| kinase 1 | | | | | | |
| Mitogen-activated | KSLVGTPYWMSPE | 3 | Q9Y2U5 | −1.34629 | 0.988065 | 0.011935 |
| protein kinase | | | | | | |
| kinase kinase 2 | | | | | | |
| Catenin beta-1 | QEYKKRLSMELTNSL | 154 | P35222 | −1.21773 | 0.987471 | 0.012529 |
| Nuclear factor NF- | KALRFRYECEGRS | 190 | P19838 | −1.71539 | 0.986429 | 0.013571 |
| kappa-B p105 subunit | | | | | | |
| Serine/threonine- | DWVFINYTFKRFEGL | 60 | Q15208 | −1.27255 | 0.986346 | 0.013654 |
| protein kinase 38 | | | | | | |
| ATP-dependent Clp | QNAMIPQYQMLFSMD | 253 | O76031 | −1.26389 | 0.985592 | 0.014408 |
| protease ATP-binding | | | | | | |
| subunit clpX-like, | | | | | | |
| mitochondrial | | | | | | |
| Proteasome subunit | VAMLMQEYTQSGGVR | 194 | P17220 | −1.33452 | 0.984875 | 0.015125 |
| alpha type-2. | | | | | | |
| Cyclin-dependent | MKKIRLESDDEGIPS | 230 | P06493 | −1.36175 | 0.984218 | 0.015782 |
| kinase 1 | | | | | | |
| Paxillin | ELDDLMASLSEFK | 138 | P49023 | −1.34708 | 0.982044 | 0.017956 |
| Cyclin-dependent | MGTVLSFSPRDRRGS | 62 | Q15078 | −1.28726 | 0.978761 | 0.021239 |
| kinase 5 activator | | | | | | |
| 1 | | | | | | |
| Sodium/potassium- | ICKTRRNSLFRQGM | 235 | P05023 | −1.49339 | 0.978448 | 0.021552 |
| transporting ATPase | | | | | | |
| subunit alpha-1 | | | | | | |
| Casein kinase 2, | ETKMSSSEEVSWIS | 48 | Q5SRQ6 | −1.42028 | 0.978 | 0.022 |
| beta polypeptide. | | | | | | |
| Glutamate dehydro- | EKITRRFTLELAKKG | 210 | P10860 | −1.35338 | 0.977443 | 0.022557 |
| genase 1, | | | | | | |
| mitochondrial | | | | | | |
| Phosphoglycerate | VQIWRRSFDTPPPPM | 191 | P18669 | −1.61808 | 0.976611 | 0.023389 |
| mutase 1 | | | | | | |
| Serine/threonine- | QHAQKETEFLRLKR | 4 | Q9Y2H1 | −1.32307 | 0.974038 | 0.025962 |
| protein kinase | | | | | | |
| 38-like | | | | | | |
| Cell division | TPNTILATPFRS | 24 | Q99459 | −1.311 | 0.972648 | 0.027352 |
| cycle 5-like | | | | | | |
| protein | | | | | | |
| Tyrosine-protein | ALKQNKFSNKSDMWS | 144 | P41240 | −1.23381 | 0.972206 | 0.027794 |
| kinase CSK | | | | | | |
| Malate dehydro- | SATLSMAYAGARFGF | 145 | P40926 | −1.47647 | 0.969745 | 0.030255 |
| genase, | | | | | | |
| mitochondrial | | | | | | |
| Nuclear inhibitor | LGLPETETELDNLTE | 53 | Q28147 | −1.15104 | 0.968963 | 0.031037 |
| of protein phos- | | | | | | |
| phatase 1 | | | | | | |
| C-C chemokine | ILHLMCISVDRYWAI | 158 | P32248 | −1.25173 | 0.967945 | 0.032055 |
| receptor type 7 | | | | | | |
| ATP synthase | LGENTVRTIAMDGTE | 112 | P56480 | −1.23135 | 0.960816 | 0.039184 |
| subunit beta, | | | | | | |
| mitochondrial | | | | | | |
| C-Jun-amino- | VMSEKVQSLAGSIY | 20 | Q9ESN9 | −1.17879 | 0.960282 | 0.039718 |
| terminal kinase- | | | | | | |
| interacting protein | | | | | | |
| 3 | | | | | | |
| Serine/threonine- | RRKSLVGTPYWMSPE | 7 | Q9VXE5 | −1.3063 | 0.951153 | 0.048847 |
| protein kinase | | | | | | |
| PAK mbt | | | | | | |
| Tyrosine-protein | RLMRDDTYTAHAGAK | 243 | P00519 | −1.22768 | 0.949686 | 0.050314 |
| kinase ABL1 | | | | | | |
| cAMP-dependent | RVQGRTWTLCGTPEY | 193 | P17612 | −1.09029 | 0.947371 | 0.052629 |
| protein kinase | | | | | | |
| catalytic subunit | | | | | | |
| alpha; | | | | | | |
| AP-1 complex sub- | VEGQDMLYQSLKLTN | 273 | O35643 | −1.16563 | 0.945242 | 0.054758 |
| unit beta-1 | | | | | | |
| ATP-binding cassette | HDLRSRLTIIPQDPV | 97 | P70170 | −1.17793 | 0.943702 | 0.056298 |
| sub-family C member | | | | | | |
| 9 | | | | | | |
| 5′-AMP-activated | VDPMKRATIEDIKKH | 76 | Q09137 | −1.21545 | 0.94369 | 0.05631 |
| protein kinase | | | | | | |
| catalytic subunit | | | | | | |
| alpha-2 | | | | | | |
| Protein kinase C beta | QTEFMGFSFLNPEFV | 232 | P05771 | −1.18618 | 0.941797 | 0.058203 |
| type; PKC-B; PKC-beta | | | | | | |
| 5′-AMP-activated | NLAAEKTYNNLDVSL | 265 | O54950 | −1.25263 | 0.94133 | 0.05867 |
| protein kinase | | | | | | |
| subunit gamma-1 | | | | | | |
| MAP kinase-activated | SNHGLAISPGMKKRI | 137 | P49137 | −1.36942 | 0.938654 | 0.061346 |
| protein kinase 2 | | | | | | |
| Mitogen-activated | TRMSAAGTYAWMAPE | 95 | P80192 | −1.21867 | 0.938282 | 0.061718 |
| protein kinase | | | | | | |
| kinase kinase 9 | | | | | | |
| Mitogen-activated | FLTEYVATRWYRAPE | 174 | P27361 | −1.27073 | 0.92928 | 0.07072 |
| protein kinase 3 | | | | | | |
| Nitric oxide | IARAVKFTSKLFGRA | 167 | P29476 | −1.21811 | 0.928372 | 0.071628 |
| synthase, brain | | | | | | |
| Mitogen-activated | TTFMMTPYVVTRYYR | 141 | P45983 | −1.22802 | 0.91886 | 0.08114 |
| protein kinase 8 | | | | | | |
| LIM domain kinase 1 | ERKKRYTVVGNPYW | 120 | P53667 | −1.14177 | 0.907635 | 0.092365 |
| Serine/threonine- | QGASGTVYTAIETST | 74 | Q13153 | −1.18873 | 0.902495 | 0.097505 |
| protein kinase PAK 1 | | | | | | |

### CITATIONS FOR REFERENCES REFERRED TO IN THE SPECIFICATION

37. S. Jalal, R. Arsenault, A. A. Potter, L. A. Babiuk, P. J. Griebel. S. Napper, Genome to Kinome: Species-Specific Arrays for Kinome Analysis. Science Signaling. Sci. Signal. 2, pl1 (2009).
38. W. Huber, A. V. Heydebreck, H. Sultmann, A. Poustka, M. Vingron, Variance stabilization applied to microarray data calibration and to the quantification of differential expression. Bioinformatics, 18 Suppl 1:S96-104 (2002).
39. R Development Core Team. R: A Language and Environment for Statistical Computing. R Foundation for Statistical Computing, Vienna, Austria. ISBN 3-900051-07-0 (2009).
40. D. C. Montgomery, Design and analysis of experiments. Hoboken, N.J.: Wiley, c2009, 7th edition (2009).
41. B. Everitt, Cluster Analysis. London: Heinemann Educ. Books (1974).
42. J. A. Hartigan, Clustering Algorithms. New York: Wiley (1975).
43. L. L. McQuitty, Similarity Analysis by Reciprocal Pairs for Discrete and Continuous Data. Educational and Psychological Measurement, 26, 825-831 (1966).
44. K. Pearson, Mathematical contributions to the theory of evolution. III. Regression, heredity and panmixia” Philos. Trans. Royal Soc. London Ser. A, 187, 253-318 (1896).
45. M. B. Eisen, P. T. Spellman, P. O. Brown, D. Botstein, Cluster analysis and display of genome-wide expression patterns. Proc. Natl. Acad. Sci. USA, 95(25):14863-8 (1998).
46. D. J. Lynn, G. L. Winsor, C. Chan, N. Richard, M. R. Laird, A. Barsky et al., InnateDB: facilitating systems-level analysis of the mammalian innate immune response. Molecular Systems Biology. 4, 218 (2008)
51. Boyle, E. I., Weng, S., Gollub, J., Jin, H., Botstein, D., Cherry, J. M., and Sherlock, G. (2004). Go::termfinder-open source software for accessing gene ontology information and finding significantly enriched gene ontology terms associated with a list of genes. Bioinformatics, 20(18), 3710-5.
53. D\check{r}aghici, S. (2003). Data analysis tools for DNA microarrays. Chapman & Hall/CRC, Boca Raton, Fla.
56. Grewal, A. and Conway, A. (2000). Tools for analyzing microarray expression data. Journal of the Association for Laboratory Automation, 5(5), 62-64.
59. Huber, W., von Heydebreck, A., Sueltmann, H., Poustka, A., and Vingron, M. (2003). Parameter estimation for the calibration and variance stabilization of microarray data. Stat Appl Genet Mol Biol, 2, Article3.
60. Kanehisa, M. and Goto, S. (2000). Kegg: kyoto encyclopedia of genes and genomes. Nucleic Acids Res, 28(1), 27-30.
61. Kanehisa, M., Goto, S., Hattori, M., Aoki-Kinoshita, K. F., Itoh, M., Kawashima, S., Katayama, T., Araki, M., and Hirakawa, M. (2006). From genomics to chemical genomics: new developments in kegg. Nucleic Acids Res, 34(Database issue), D354-7.
62. Kanehisa, M., Goto, S., Furumichi, M., Tanabe, M., and Hirakawa, M. (2010). Kegg for representation and analysis of molecular networks involving diseases and drugs. Nucleic Acids Res, 38(Database issue), D355-60.
121. Gentleman et al. Missing from the list of references. Genome Biology 2004; 5(10):R80.
F Diella, S Cameron, C Gem und, R Linding, A Via, B Kuster, T Sicheritz-Pont_en, N Blom, and T J Gibson.
Phospho.ELM: a database of experimentally veri_ed phosphorylation sites in eukaryotic proteins.
BMC
Bioinformatics, 5:79, 2004. doi: 10.1186/1471-2105-5-79.
F Diella, C M Gould, C Chica, A Via, and T J Gibson. Phospho.ELM: a database of phosphorylation sites{update 2008. Nucleic Acids Res, 36(Database issue):D240{4, 2008. doi: 10.1093/nar/gkm772.

1. (canceled)

2. (canceled)

3. An array comprising a support and i) a plurality of peptides each peptide of the plurality comprising a sequence of about 5 to about 100 amino acids, for example about 5 to about 50 amino acids or about 5 to about 30 amino acids, wherein the sequence comprises a contiguous sequence present in a peptide sequence selected from the group of SEQ ID NOs: 1 to 288, wherein the contiguous sequence comprises a bee phosphorylation site sequence and/or ii) a plurality of bee species peptides, each peptide comprising a sequence of about 5 to about 50 amino acids, about 5 to about 30 amino acids or about 8 to about 15 amino acids, wherein the peptide sequence comprises a phosphorylation site sequence.

4. The array of claim 3, wherein each sequence is 8-15 amino acids of a peptide sequence selected from SEQ ID NO: 1-288.

5. The array of claim 3 comprising a plurality of peptides each peptide comprising a peptide sequence selected from the group listed in Table 2, 3, and/or 4.

6. The array of claim 3, wherein each peptide is spotted on the support in duplicate, triplicate or more.

7. The array of claim 4, wherein the plurality of peptides comprises at least 25, 50, 75, 100, 125, 150, 200, 250 or at least 288 different peptides.

8. A method for measuring protein kinase activity in a sample from a subject, said method comprising the steps of:
a) obtaining the sample from the subject;
b) incubating said sample with:
i) ATP or other suitable ATP analog;
ii) a plurality of peptides,
I) the array of claim 3; or
II) each peptide of the plurality comprising a sequence of about 5 to about 100 amino acids, for example about 5 to about 50 amino acids or about 5 to about 30 amino acids, wherein the sequence comprises a contiguous sequence present in a peptide sequence selected from the group of SEQ ID NOs: 1 to 288, wherein the contiguous sequence comprises a bee phosphorylation site sequence, and
c) determining a detectable phosphorylation profile, said phosphorylation profile resulting from the interaction of the sample with the plurality of peptides
wherein the detectable phosphorylation profile provides a measure of the protein kinase activity in the sample.

9. (canceled)

10. The method of claim 8 for identifying a biomarker and/or set of biomarkers in a subject associated with a desirable phenotype, the method further comprising:
d) comparing the phosphorylation profile of the sample with a control;
wherein a difference or a similarity in the phosphorylation profile of the plurality of peptides between the sample and the control is used to identify the biomarker and/or set of biomarkers associated with the desirable phenotype.

11. The method of claim 10, wherein the subject is subjected to a stressor prior to obtaining the sample.

12. The method of claim 11, wherein the stressor is a pathogen challenge.

13. (canceled)

14. A method of classifying a subject, the method comprising:
a) determining a detectable phosphorylation profile of a sample obtained from the subject, said phosphorylation profile resulting from the interaction of the sample with a plurality of peptides each peptide of the plurality comprising a sequence of about 5 to about 100 amino acids, for example about 5 to about 50 amino acids or about 5 to about 30 amino acids, wherein the sequence comprises a contiguous sequence present in a peptide sequence selected from the group of SEQ ID NOs: 1 to 288, and wherein the contiguous sequence comprises a bee phosphorylation site sequence;
b) comparing said phosphorylation profile to a reference phosphorylation profile of a known phenotype and
c) classifying the subject according to the probability of said phosphorylation profile falling within a class defined by said reference phosphorylation profile.

15. (canceled)

16. A method of phenotyping a subject or screening a subject for susceptibility and/or resistance to a pathogen, the method comprising:
a) obtaining a sample from the subject;
b) contacting the sample with ATP and/or a suitable ATP analog;
i) the array of claim 3; or
ii) a plurality of peptides each peptide of the plurality comprising a sequence of about 5 to about 100 amino acids, for example about 5 to about 50 amino acids or about 5 to about 30 amino acids, wherein the sequence comprises a contiguous sequence present in a peptide sequence selected from the group of SEQ ID NOs: 1 to 288, wherein the contiguous sequence comprises a bee phosphorylation site sequence;
c) determining a phosphorylation profile of the plurality of peptides;
d) comparing the phosphorylation profile of the plurality of peptides with one or more reference phosphorylation profiles;
e) identifying the subject as having or not having the phenotype or as being susceptible or resistant to the pathogen according to a
difference or a similarity in the phosphorylation profile between the sample and the one or more reference phosphorylation profiles.

17. A method of aiding selection of a subject with a desirable phenotype comprising:
a) determining a subject phosphorylation profile from a sample obtained from the subject;
b) providing one or more reference phosphorylation profiles associated with a known phenotype, wherein the subject phosphorylation profile and the reference phosphorylation profile(s) have one or a plurality of values, each value representing a phosphorylation level of a peptide selected from a plurality of peptides each peptide of the plurality comprising a sequence of about 5 to about 100 amino acids, for example about 5 to about 50 amino acids or about 5 to about 30 amino acids, wherein the sequence comprises a contiguous sequence present in a peptide sequence selected from the group of SEQ ID NOs: 1 to 288, and wherein the contiguous sequence comprises a bee phosphorylation site sequence; and
c) identifying the reference phosphorylation profile most similar to the subject phosphorylation profile,
wherein the subject is predicted to have the phenotype of the reference phosphorylation profile most similar to the subject phosphorylation profile.

18. The method of claim 17, wherein the phosphorylation level of the peptide is obtained using the corresponding protein.

19. The method of claim 17 for screening for varroa resistance or Nosema resistance.

20. The method of claim 19, wherein the subject is infected with varroa prior to obtaining the sample and decreased phosphorylation, relative to an uninfected subject, of two or more peptides in Table 2A and/or 3A is indicative that the subject is varroa resistant and/or increased phosphorylation, relative to an uninfected subject, of two or more peptides in Table 2B and/or 3B is indicative that the subject is varroa resistant.

21. The method of claim 19, wherein the subject is uninfected with varroa and decreased phosphorylation, relative to a varroa-sensitive subject, of two or more peptides in Table 2A and/or 4A is indicative that the subject is varroa resistant and/or increased phosphorylation of two or more peptides in Table 2B and/or 4B, relative to a varroa-sensitive subject, is indicative that the subject is varroa resistant.

22. (canceled)

23. The method of claim 8, wherein the subject is a bee, optionally a honey bee.

24-27. (canceled)

28. The method of claim 8, wherein the step of determining a phosphorylation profile comprises:
a) obtaining one or more datasets, each dataset comprising a phosphorylation signal intensity for each peptide of the plurality of peptides;
b) transforming the phosphorylation signal intensity of each peptide of the plurality of peptides using a variance stabilizing transformation to provide a variance stabilized signal intensity for each peptide of the plurality of peptides; and
c) identifying one or more peptides of the plurality of peptides that are consistently phosphorylated or consistently unphosphorylated,
thereby providing a subject phosphorylation profile.

29. A kit comprising:
i) a plurality of peptides each peptide of the plurality which comprises a sequence of about 5 to about 100 amino acids, for example about 5 to about 50 amino acids or about 5 to about 30 amino acids, wherein the sequence comprises a contiguous sequence present in a peptide sequence selected from the group of SEQ ID NOs: 1 to 288, wherein the contiguous sequence comprises a bee phosphorylation site sequence; and/or
ii) the array of claim 3;
iii) optionally in combination with a kit control;
iv) and a package housing the peptides and/or an array and/or kit control.

30. The method of claim 16, wherein the step of determining a phosphorylation profile comprises:
a) obtaining one or more datasets, each dataset comprising a phosphorylation signal intensity for each peptide of the plurality of peptides;
b) transforming the phosphorylation signal intensity of each peptide of the plurality of peptides using a variance stabilizing transformation to provide a variance stabilized signal intensity for each peptide of the plurality of peptides; and
c) identifying one or more peptides of the plurality of peptides that are consistently phosphorylated or consistently unphosphorylated,
thereby providing a subject phosphorylation profile.
